(19) United States
(12) Patent Application Publication
(10) Pub. No.: US 2022/0071960 A1
(43) Pub. Date: Mar. 10, 2022

(54) CANCER TREATMENTS

(71) Applicant: IMPERIAL COLLEGE INNOVATIONS LIMITED, London (GB)

(72) Inventors: Edward William TATE, London (GB); Monica FARONATO, London (GB); Dinis CALADO, London (GB); Gregor LUEG, London (GB)

(73) Assignee: IMPERIAL COLLEGE INNOVATIONS LIMITED, London (GB)

(21) Appl. No.: 17/414,697

(22) PCT Filed: Dec. 19, 2019
(86) PCT No.: PCT/GB2019/053615

§ 371 Date: Jun. 16, 2021

(30) Foreign Application Priority Data
Dec. 19, 2018 (GB) 1820660.7

(51) Int. Cl. A61K 31/416; A61K 31/437; A61K 31/496; A61P 35/00

(52) U.S. Cl. CPC A61K 31/416; A61P 35/00; A61K 31/496; A61K 31/437

(57) ABSTRACT

The present invention relates to the use of compounds which display activity as inhibitors of the human N-myristoyl transferases (NMT) in the treatment of MYC addicted cancers, such as, for example, cancers comprising MYC overexpression. The present invention also relates to the use of compounds which display activity as inhibitors of NMT, in combination with one or more other therapeutic agents, in the treatment of MYC addicted cancers and/or MYC dysregulated cancers.

### INTRODUCTION

The present invention relates to the use of compounds which display activity as inhibitors of the human N-myristoyl transferases (NMT) in the treatment of MYC addicted cancers, such as, for example, cancers comprising MYC overexpression. The present invention also relates to the use of compounds which display activity as inhibitors of NMT, in combination with one or more other therapeutic agents, in the treatment of MYC addicted cancers and/or MYC dysregulated cancers.

### BACKGROUND OF THE INVENTION

Hyperproliferative disorders, such as cancer, typically evolve though a multistage process, in which the growth and proliferation of the cancerous cell is driven by several progressive genetic mutations and epigenetic abnormalities (Weinstein et al., Cancer Res., 2008, 68(9), 3077-3080). This multifaceted mode of action and, in particular, the simultaneous activation of oncogenes and deactivation of tumour suppressor genes makes the task of developing effective cancer treatments extremely difficult (Sharma et al., Genes and Development, 2007, 21, 3214-3231). In fact, cancer is often described as a “moving target” due to its progressive nature.

Many forms of cancer, such as, for example, myelomas, lymphomas, leukaemias, neuroblastomas and certain solid tumours (e.g. breast cancers), can be extremely complex and challenging to treat. Remissions and relapses as a result of resistance to chemotherapy in subjects can be fatal, and survival times for subjects who develop a resistance to common forms of chemotherapy are typically very short. There therefore remains a need for new and improved methods for treating cancers, together with improved methods for enhancing the efficacy of current chemotherapies, particularly in those subjects resistant, refractory, or otherwise not responsive to treatment with such chemotherapies.

“Oncogene addiction”, first coined by Bernard Weinstein in 2008, is a term used to describe the absolute dependence of some cancers on one or only a few genes for maintaining a malignant phenotype, and this is an area of research currently being explored for its prospects in providing new and effective cancer treatments (Weinstein et al., Cancer Res., 2008, 68(9), 3077-3080). Within this particular field of research, the identification of “transcriptionally addicted” cancers, which are cancers having an absolute reliance on certain transcriptional factors, has recently yielded some promising leads for new treatments (Bradner et al., Cell, 2017, 168, 629-643). One family of transcriptional factors of particular interest is that of the MYC regulator genes.

MYC regulator genes encode a family of transcription factors involved in cell proliferation, growth, differentiation and apoptosis. Members of the MYC transcription factor family include c-MYC, MYCN and MYCL, sometimes referred to as c-Myc, N-Myc and L-Myc, (Kalkat, et al., 2013, Regulation and Function of the MYC Oncogene, John Wiley & Sons Ltd). Activation of normal MYC genes affects numerous cellular processes, including cell cycle progression, cell growth and division, metabolism, telomerase activity, adhesion and motility, angiogenesis and differentiation. MYC, has further been identified as a strong proto-oncogene and its mutated versions are often found to be upregulated and/or constitutively expressed in certain types of cancers, such as haematological cancers and solid tumour malignancies (Miller et al., Clin. Cancer Res., 2012, 18(20), 5546-5553). As an example, c-MYC overexpression has been observed in up to 30% of cases of diffuse large B-cell lymphoma (DLBCL), the most common type of aggressive lymphoma (Chisholm et al., Am. J. Surg. Pathol., 2015, 39(3), 294-303). Furthermore, around 15% of breast tumours have been shown to be overexpressant in MYC (Xu et al., Genes and Cancer, 2010, 1 (6), 629-640), and, further still, around 20% of human renal cell adenocarcinomas (RCC) have been identified as overexpressing in MYC (Shroff et al., Cell, 2015, 112(21), 6539-6544). Moreover, mutations and/or copy number gains of the MYC gene are observed in roughly 28% of all cancers listed in “The Cancer Genone Atlas” (TCGA) database (Schaub et al., Cell Systems, 2018, 6, 282-300).

However, despite the attractiveness of targeting the MYC oncogene and exploiting the vulnerability of MYC addiction shown by specific cancers, the development of effective treatments for MYC addicted cancers has been slow, and currently no drugs or methods of treatment that directly target MYC overexpressed cancers have been approved for use.

Thus, there remains a need for new strategies and treatments which are able to treat patients with transcriptionally addicted cancers and, in particular, MYC addicted cancers.

The present invention was devised with the foregoing in mind.

### SUMMARY OF THE INVENTION

According to a first aspect of the present invention, there is provided a NMT inhibitor, or a pharmaceutically acceptable salt, solvate or hydrate thereof, for use in the treatment of a MYC addicted cancer.

According to a further aspect of the present invention, there is provided a NMT inhibitor, or a pharmaceutically acceptable salt, solvate or hydrate thereof, for use in the treatment of a MYC dysregulated cancer.

According to a further aspect of the present invention, there is provided a NMT inhibitor, or a pharmaceutically acceptable salt, solvate or hydrate thereof, for use in the treatment of a MYC overexpressing cancer.

According to a further aspect of the present invention, there is provided a NMT inhibitor, or a pharmaceutically acceptable salt, solvate or hydrate thereof, for use in the treatment of a cancer comprising one or more structural alterations of the MYC locus (e.g. a cancer comprising a mutated MYC oncogene).

According to a further aspect of the present invention, there is provided a NMT inhibitor, or a pharmaceutically acceptable salt, solvate or hydrate thereof, for use in combination with one or more other therapeutic agents in the treatment of a MYC addicted cancer.

According to a further aspect of the present invention, there is provided a NMT inhibitor, or a pharmaceutically acceptable salt, solvate or hydrate thereof, for use in combination with one or more other therapeutic agents in the treatment of a MYC dysregulated cancer.

According to a further aspect of the present invention, there is provided a NMT inhibitor, or a pharmaceutically acceptable salt, solvate or hydrate thereof, for use in combination with one or more other therapeutic agents in the treatment of a MYC overexpressing cancer.

According to a further aspect of the present invention, there is provided a NMT inhibitor, or a pharmaceutically acceptable salt, solvate or hydrate thereof, for use in combination with one or more other therapeutic agents in the treatment of a cancer comprising one or more structural alterations of the MYC locus (e.g. a cancer comprising a mutated MYC oncogene).

According to a further aspect of the present invention, there is provided a method for the treatment of a MYC addicted cancer in a subject in need of such treatment, said method comprising administering a therapeutically effective amount of a NMT inhibitor, or a pharmaceutically acceptable salt, solvate or hydrate thereof.

According to a further aspect of the present invention, there is provided a method for the treatment of a MYC dysregulated cancer in a subject in need of such treatment, said method comprising administering a therapeutically effective amount of a NMT inhibitor, or a pharmaceutically acceptable salt, solvate or hydrate thereof.

According to a further aspect of the present invention, there is provided a method for the treatment of a MYC overexpressing cancer in a subject in need of such treatment, said method comprising administering a therapeutically effective amount of a NMT inhibitor, or a pharmaceutically acceptable salt, solvate or hydrate thereof.

According to a further aspect of the present invention, there is provided a method for the treatment of a cancer comprising one or more structural alterations of the MYC locus in a subject in need of such treatment, said method comprising administering a therapeutically effective amount of a NMT inhibitor, or a pharmaceutically acceptable salt, solvate or hydrate thereof.

According to a further aspect of the present invention, there is provided a method for determining whether a subject with a cancer will benefit from treatment with an NMT inhibitor, said method comprising the steps of:
i) taking a sample of cancer cells taken from said subject;
ii) analysing the cancer cells of step i) to check for the presence of one or more structural alterations in the MYC locus (e.g. chromosomal rearrangements, copy number gains and/or mutations of the MYC oncogene);
iii) determining whether one or more structural alterations (e.g. chromosomal rearrangements, copy number gains and/or mutations) are present in the MYC locus of the sample of cancer cells when compared to a control; and
iv) determining whether the subject will benefit from being administered a NMT inhibitor in order to treat said cancer, wherein if the sample of cancer cells contains one or more structural alterations (e.g. chromosomal rearrangements, copy number gains and/or mutations) in the MYC locus, then the subject will benefit from being administered a NMT inhibitor, and if the sample of cancer cells does not contain one or more structural alterations (e.g. chromosomal rearrangements, copy number gains and/or mutations) in the MYC locus, then the subject will not benefit from being administered a NMT inhibitor.

According to a further aspect of the present invention, there is provided a method for determining whether a subject with a cancer will benefit from treatment with an NMT inhibitor, said method comprising the steps of:
i) measuring the level of MYC expression in a sample of cancer cells taken from said subject;
ii) comparing the level of MYC expression from step i) with a control;
iii) determining whether the MYC expression in the sample of cancer cells is increased compared to the control; and
iv) determining whether the subject will benefit from being administered a NMT inhibitor in order to treat said cancer, wherein if the MYC expression in the sample of cancer cells is higher than in the control, then the subject will benefit from being administered a NMT inhibitor, and if the MYC expression in the sample of cancer cells is not higher than in the control, then the subject will not benefit from being administered a NMT inhibitor.

According to a further aspect of the present invention, there is provided a method for the treatment of cancer in a subject who has been identified as benefiting from being administered a NMT inhibitor as determined by a method as described herein, wherein said method comprises administering a therapeutically effective amount of a NMT inhibitor, or a pharmaceutically acceptable salt, solvate or hydrate thereof to the subject.

Features, including optional, suitable, and preferred features in relation to one aspect of the invention may also be features, including optional, suitable and preferred features in relation to any other aspect of the invention.

### BRIEF DESCRIPTION OF THE DRAWINGS

FIG. 1 shows the representative flow cytometry analysis for the expression of c-MYC and the cell size in the P-493-6 cell line.

FIG. 2 shows the representative flow cytometry analysis of EdU incorporation, clicked to an AlexaFluor 488 fluorochrom.

FIG. 3 shows the quantification of the relative proportion of cells that are EdU positive and the cell number in a 100 uL of cell suspension.

FIG. 4 shows metabolic viability of the P-493-6 cell line treated with Compound 2 at the shown concentrations for 48 hours and 72 hours, with different expression levels of c-MYC induced.

FIG. 5 shows the metabolic viability of the P-493-6 cell line treated with Compound 6, Compound 5 and Compound 1 at the shown concentrations for 72 hours, with different expression levels of c-MYC induced.

FIG. 6 shows the cell quantification data for the P-493-6 cell line with high c-MYC expression.

FIG. 7 shows the cell quantification data for the P-493-6 cell line with medium c-MYC expression.

FIG. 8 shows the cell quantification data for the P-493-6 cell line with low c-MYC expression.

FIG. 9 shows the cell cycle quantification for the P-493-6 cell line at 48 hours and 72 hours with medium and high c-MYC expression.

FIG. 10 shows the representative western blot showing MYCN protein expression following time course treatment with 100 nM of Tamoxifen in the SKNAS-ER-MYCN and Shep-ER-MYCN cell lines.

FIG. 11 shows the representative flow cytometric analysis of the cell cycle profile for the Shep-ER-MYCN cell line, with and without induction of MYCN.

FIG. 12 shows the representative flow cytometric analysis of the cell cycle profile for the SKNAS-ER-MYCN cell line, with and without induction of MYCN.

FIG. 13 shows the representative flow cytometric analysis of the protein synthesis, via incorporation of OPP, of the Shep-ER-MYCN and SKNAS-ER-MYCN cell lines with or without induction of MYCN.

FIG. 13 shows the metabolic viability of the Shep-ER-MYCN and SKNAS-ER-MYCN cell lines treated with Compound 2 at the shown concentrations for 48 hours and 72 hours, with and without induction of MYCN.

FIG. 14 shows the metabolic viability of the Shep-ER-MYCN cell line treated with Compounds 1, 2 and 6 at the shown concentrations for 72 hours, with and without induction of MYCN.

FIG. 15 shows the metabolic viability of the SKNAS-ER-MYCN cell line treated with Compounds 1, 2 and 6 at the shown concentrations for 92 hours, with and without induction of MYCN.

FIG. 16 shows the cell quantification data for the Shep-ER-MYCN cell line without and with MYCN induction upon administration of Compound 2.

FIG. 17 shows the quantification of the cell cycle profile of the Shep-ER-MYCN cell line with and without induction of MYCN upon administration of Compound 2 at 48 and 72 hours.

FIG. 18 shows the cell quantification data for the SKNAS-ER-MYCN cell line without and with MYCN induction upon administration of Compound 2. FIG. 19 shows the quantification of the cell cycle profile of the SKNAS-ER-MYCN cell line without and with MYCN induction upon administration of Compound 2.

FIG. 20 shows the global comparison of the correlation between sensitivity to the NMT inhibitor Compound 6, measured as EC50, and normalised c-MYC expression.

FIG. 21 shows the global comparison of the correlation between sensitivity to the NMT inhibitor Compound 4, measured as EC50, and normalised c-MYC expression.

FIG. 22 shows the global comparison of the correlation between sensitivity to the NMT inhibitor Compound 3, measured as EC50, and normalised c-MYC expression.

FIG. 23 shows the global comparison of the correlation between sensitivity to the NMT inhibitors Compound 6, Compound 4 and Compound 3, measured as EC50 and normalised NMT1 and NMT2 expression.

FIG. 24 shows the effect of 100 nM Compound 2 for 24 hours on different MYC gene sets, measured via RNAseq in BL41 cells.

FIG. 25 shows the effect of 100 nM Compound 2 for 24 hours on different MYC gene sets, measured via RNAseq in HeLa cells.

FIG. 26 shows the effect on the sensitivity, measured as EC50, of the presence of structural alterations in the genomic loci of c-MYC, MYCN and MYCL for Compound 4, Compound 3 and Compound 6.

FIG. 27 shows the activation of different MYC gene sets if structural alterations in the genomic loci of c-MYC, MYCN and MYCL are present.

FIG. 28 shows the GSEA analysis on the cancer cell line screen with Compound 4

FIG. 29 shows the leading-edge analysis on the cancer cell line screen with Compound 4, and the generation of a gene set ‘Sensitive to NMTi’.

FIG. 30 shows the application of the gene set ‘Sensitive to NMTi’ on a gene knock out essentiality screen and its upregulation in cancers with high c-Myc expression and/or structural alterations in the loci of c-MYC/MYCN/MYCL.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “cancer” used herein will be understood to mean any neoplastic growth in a subject, including an initial tumour and any metastases. The cancer can be of the liquid or solid tumour type. Liquid tumours include, for example, tumours of haematological origin (haematological cancer), including, e.g., myelomas (e.g., multiple myeloma), leukaemias (e.g., Waldenstrom's syndrome, chronic lymphocytic leukemia, other leukaemias), and lymphomas (e.g., B-cell lymphomas). Solid tumours can originate in organs, and include cancers such as lung, breast, prostate, ovary, colon, kidney, and liver.

The term “cancerous cell” or “cancer cell” used herein will be understood to mean a cell that shows aberrant cell growth, such as increased cell growth. A cancerous cell may be a hyperplastic cell, a cell that shows a lack of contact inhibition of growth in vitro, a tumour cell that is incapable of metastasis in vivo, or a metastatic cell that is capable of metastasis in vivo. Cancer cells include, but are not limited to, carcinomas, such as myelomas, leukaemias (e.g., chronic lymphocytic leukaemia, myeloid leukaemia and B-acute lymphocytic leukaemia), lymphomas (e.g., follicular lymphoma, mantle cell lymphoma, diffuse large B-cell lymphoma or Hodgkins disease) and blastomas (e.g. retinoblastoma or glioblastoma).

The term “MYC” used herein will be understood to mean the whole family of regulator genes (transcriptional factors) that fall within the MYC gene family. Members of the MYC transcription factor family include c-MYC, MYCN and MYCL, and thus reference to “MYC” herein will be understood to covers all of such family members. The non-italicised terms “MYC”, “c-MYC”, “MYCN” or “MYCL” will, unless stated otherwise, be understood to mean the proteins derived from the corresponding MYC gene. For example, a reference to “MYCN” will be understood to mean the protein derived from the “MYCN” gene. Such nomenclature is consistent with the nomenclature used in the art. In certain instances, the terms “MYC”, “c-MYC”, “MYCN” and “MYd” may be used synonymously with the respective uncapitalised terms “Myc”, “c-Myc”, “N-Myc” and “L-Myc”.

The terms “overexpressed”, “overexpression” and “overexpressing” used herein may be taken to mean the expression (i.e. level/amount) of mRNA and/or protein found in a particular cell (i.e. cancer cell) is elevated compared to the expression (i.e. level/amount) of mRNA and/or protein found in a normal, healthy cell (i.e. a cancer-free cell). Thus, for example, a “MYC overexpressing cancer” will be understood to be a cancer which expresses elevated RNA transcript and/or protein levels of MYC compared to the RNA transcript and/or protein levels of MYC found in normal, healthy cells. Suitably, the MYC overexpressing cancer is a cancer wherein the levels of RNA transcript and/or protein of MYC are at least 25% greater than the RNA transcript and/or protein levels of MYC in a normal, healthy cell. More suitably, the MYC overexpressing cancer is a cancer wherein the levels of RNA transcript and/or protein of MYC are at least 50% greater than the RNA transcript and/or protein levels of MYC in a normal, healthy cell. Even more suitably, the MYC overexpressing cancer is a cancer wherein the levels of RNA transcript and/or protein of MYC are at least 100% greater than the RNA transcript and/or protein levels of MYC in a normal, healthy cell. Yet more suitably, the MYC overexpressing cancer is a cancer wherein the levels of RNA transcript and/or protein of MYC are at least 200% greater than the RNA transcript and/or protein levels of MYC in a normal, healthy cell. Most suitably, the MYC overexpressing cancer is a cancer wherein the levels of RNA transcript and/or protein of MYC are at least 400% greater than the RNA transcript and/or protein levels of MYC in a normal, healthy cell. The level of expression may be determined by any suitable means known in the art. For example, the level of expression of MYC may be determined by measuring MYC protein levels. The MYC protein levels may be measured using any suitable technique known in the art, such as, for example, SDS-PAGE followed by Western blot using suitable antibodies raised against the target protein. In addition, or alternatively, the level of expression of MYC may be determined by measuring the level of mRNA. The level of mRNA may be measured using any suitable technique known in the art, such as, for example, northern blot, quantitative RT-PCR (qRT-PCR) or immunohistochemical (IHC), see for example Kluk et al., PLos One, 2012, 7(4), 1-9.

The terms “transcriptional addiction” and “transcriptionally addicted” used herein are terms of the art and may be understood to refer to cancers, and more specifically cancer cells, that exhibit a dependence on the transcription of a particular oncogene for continued growth and proliferation. In such “transcriptionally addicted” cancers, reducing the transcription of the oncogene, or inhibiting the oncogene which the cancer cells are transcriptionally addicted to, results in significant apoptosis and/or differentiation of said cancer cells. Thus, it will be understood that the terms “MYC addiction” and “MYC addicted” refer to cancers, and more specifically cancer cells, that exhibit a dependence on the MYC oncogene for continued growth and proliferation.

The term “MYC dysregulated cancer” used herein will be understood to mean a cancer in which one or more of the regulation mechanisms of the MYC oncogene are altered (e.g. mutated) or stabilised. The term “regulation mechanisms” will further be understood to mean any normal process of the MYC gene such as, for example, transcription or translation. Thus, it will be understood that the term “MYC dysregulated cancer” suitably covers both: i) mutations to the MYC oncogene which result in, for example, overexpression of the protein/mRNA of MYC; and ii) mutations to the MYC oncogene which result in, for example, a stabilisation of the protein/mRNA of MYC.

The term “locus” used herein will be understood to mean the location of a gene on a chromosome. Thus, the term “MYC locus” will be understood to mean the position on a chromosome corresponding to the MYC gene. Hence, the term “one or more structural alterations of the MYC locus” will be readily understood to mean that one or more alterations (e.g. mutations, amplifications and/or chromosomal rearrangements) are present at a location of the chromosome which corresponds to the MYC gene.

The term “hydrocarbyl” used herein will be understood to mean any compound straight or branched chain saturated, unsaturated or partially unsaturated hydrocarbon groups. Suitable examples of “hydrocarbyl” groups may include, for example, “alkyl”, “alkenyl”, “alkynyl” and/or “haloalkyl” groups, each of which are as defined hereinbelow.

The term “alkyl” used herein will be understood to mean straight and branched chain saturated hydrocarbon groups. Examples of “alkyl” groups include methyl, ethyl, n-propyl, iso-propyl, n-butyl, t-butyl, i-butyl, sec-butyl, pentyl and hexyl groups. Among unbranched alkyl groups, there are preferred methyl, ethyl, n-propyl, iso-propyl, n-butyl groups. Among branched alkyl groups, there may be mentioned t-butyl, i-butyl, 1-ethylpropyl and 1-ethylbutyl groups.

The term “Cₘ₋ₙ” or “(m-nC) group” used alone or as a prefix, refers to any group having m to n carbon atoms.

As used herein, the term “alkenyl” means both straight and branched chain unsaturated hydrocarbon groups with at least one carbon carbon double bond. Examples of alkenyl groups include ethenyl, propenyl, butenyl, pentenyl and hexenyl. Preferred alkenyl groups include ethenyl, 1-propenyl, 2-propenyl and but-2-enyl.

As used herein, the term “alkynyl” means both straight and branched chain unsaturated hydrocarbon groups with at least one carbon carbon triple bond. Examples of alkynyl groups include ethynyl, propynyl, butynyl, pentynyl and hexynyl. Preferred alkynyl groups include ethynyl, 1-propynyl and 2-propynyl.

As used herein, the term “carbocyclyl” (or “carbocycle”) is intended to mean any 3- to 13-membered carbon ring system, which may be saturated, partially unsaturated, or aromatic. The carbon ring system may be monocyclic or contain more than one ring (e.g. the ring system may be bicyclic). Examples of monocyclic saturated carbocycles include cyclopropyl, cyclobutyl, cyclopentyl, cyclohexyl, cycloheptyl, cyclooctyl. Examples of bicyclic saturated carbocycles include bicyclooctane, bicyclononane, bicyclodecane (decalin) and bicyclooctane. A further example of a saturated carbocycle is adamantane. Examples of monocyclic non-saturated carbocycles include cyclobutene, cyclopentene, cyclopentadiene, cyclohexene. Examples of aromatic carbocycles include phenyl and naphthyl. Further examples of aromatic carbocycles include tetrahydronaphthyl (tetralin) and indane.

As used herein, the term “cycloalkyl” means a saturated group in a ring system. A cycloalkyl group can be monocyclic or bicyclic. A bicyclic group may, for example, be fused or bridged. Examples of monocyclic cycloalkyl groups include cyclopropyl, cyclobutyl and cyclopentyl. Other examples of monocyclic cycloalkyl groups are cyclohexyl, cycloheptyl and cyclooctyl. Examples of bicyclic cycloalkyl groups include bicyclo [2. 2.1]hept-2-yl. Preferably, the cycloalkyl group is monocyclic.

As used herein, the term “halogen” or “halo” means fluorine, chlorine, bromine or iodine. Fluorine, chlorine and bromine are particularly preferred.

As used herein, the term “haloalkyl” means an alkyl group having a halogen substituent, the terms “alkyl” and “halogen” being understood to have the meanings outlined above. Similarly, the term “dihaloalkyl” means an alkyl group having two halogen substituents and the term “trihaloalkyl” means an alkyl group having three halogen substituents. Examples of haloalkyl groups include fluoromethyl, chloromethyl, bromomethyl, fluoromethyl, fluoropropyl and fluorobutyl groups; examples of dihaloalkyl groups include difluoromethyl and difluoroethyl groups; examples of trihaloalkyl groups include trifluoromethyl and trifluoroethyl groups.

As used herein, the term “heterocyclyl” (or heterocycle) means an aromatic or a non-aromatic cyclic group of carbon atoms wherein from one to four of the carbon atoms is/are replaced by one or more heteroatoms independently selected from nitrogen, oxygen or sulfur. A heterocyclyl (or heterocycle) group may, for example, be monocyclic or bicyclic. In a bicyclic heterocyclyl (or heterocycle) group there may be one or more heteroatoms in each ring, or only in one of the rings. A heteroatom may be S, O or N, and is preferably O or N.

Examples of monocyclic non-aromatic heterocyclyl (or heterocycle) include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl.

Examples of monocyclic aromatic heterocyclyl (or heterocycle) groups include furanyl, thienyl, pyrrolyl, oxazolyl, thiazolyl, imidazolyl, oxadiazolyl, thiadiazolyl, pyridyl, triazolyl, triazinyl, tetrazolyl, pyridazyl, isothiazolyl, isoxazolyl, pyrazinyl, pyrazolyl and pyrimidinyl.

Examples of bicyclic aromatic heterocyclyl groups (or heterocycle) include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridiyl, pyridopyrimidinyl, isoquinolinyl and benzodroxazole. Further examples of bicyclic aromatic heterocyclyl groups include those in which one of the rings is aromatic and the other is non-aromatic, such as dihydrobenzofuranyl, indanyl, indolinyl, isoindolinyl, tetrahydroisoquinolinyl, tetrahydroquinolyl and benzoazepanyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups, it is to be understood that this definition includes all substituents being chosen from one of the specified groups or the substituents being chosen from two or more of the specified groups.

The phrase “NMT inhibitor of the invention” means those compounds which display activity as inhibitors of the human N-myristoyl transferases (NMT) which are disclosed herein, both generically and specifically.

### Therapeutic Uses and Applications

To their surprise, the inventors of the present invention have found that upon administering a NMT inhibitor to a cancer with overexpressed levels of the MYC oncogene, and/or to a cancer with one or more structural alterations in the MYC locus (e.g. one or more chromosomal rearrangements or mutations), a significant improvement in both the kill rate and time to kill of the cancer, together with a lowering of the concentration of NMT inhibitor needed to adversely effect the cell is achieved compared to administration of a NMT inhibitor to a cancer without overexpressed levels of the MYC oncogene.

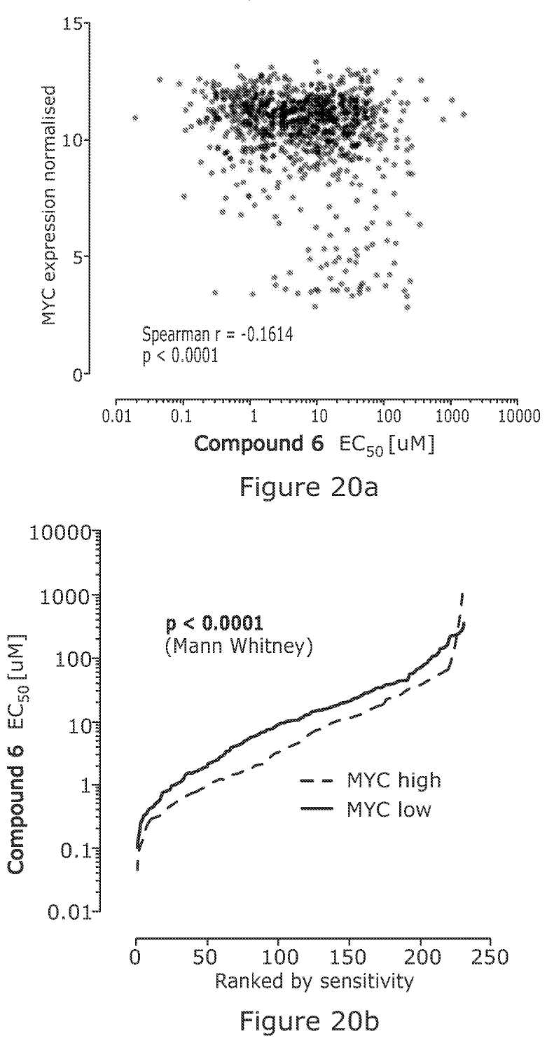

Following much investigation, the inventors discovered there to be a significant correlation between the mRNA expression levels of, for instance, c-MYC and the responsiveness to an NMT inhibitor in three pharmacogenomics screens (see, for example, FIGS. 20 to 22). This correlation was observed using two different types of analysis (Spearman correlation between c-Myc expression and EC50, and dividing cells by high and low expression of c-Myc and comparing both groups) and was found to be observed for a selection of structurally diverse NMT inhibitors in different types of cancer cell lines (e.g. 676 to 913 cancer cell lines).

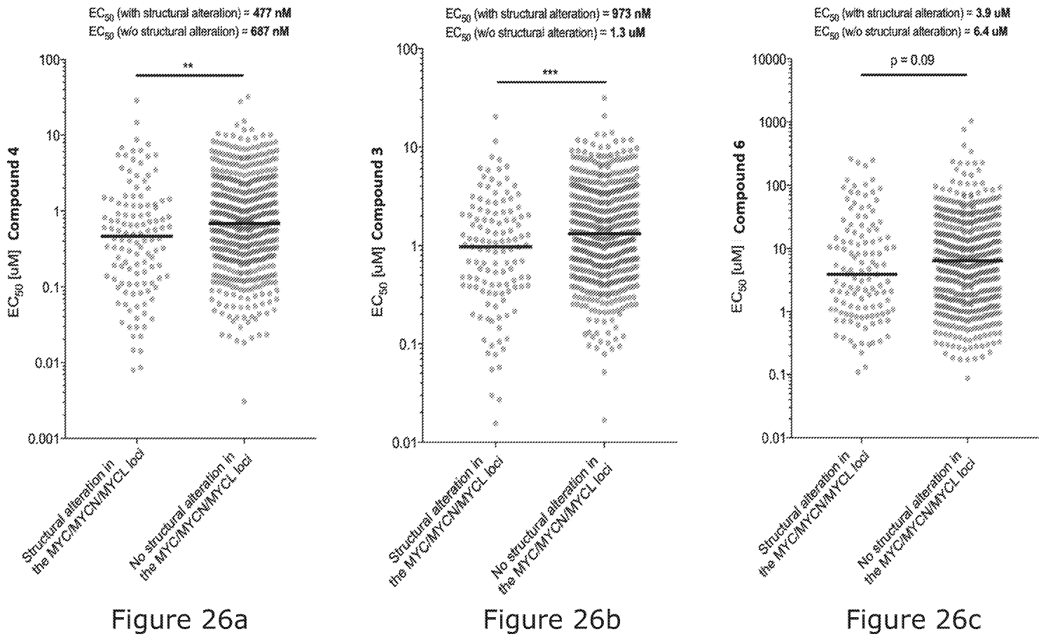

Furthermore, the inventors also observed that one or more structural alterations in the MYC locus (e.g. MYCN and MYCL) correlated significantly with an increased sensitivity to an NMT inhibitor. These observations were again consistent across three pharmacogenomics screens, and upon using a selection of structurally diverse NMT inhibitors and different cancer cell lines (676 cancer cell lines). See, for example, FIG. 26.

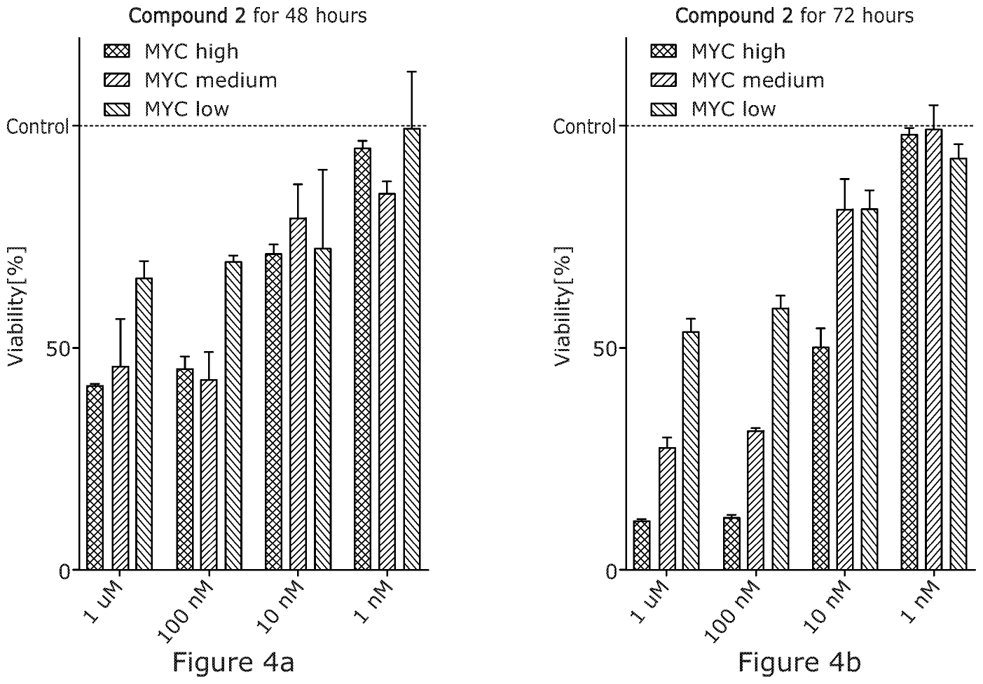

In addition to these strong correlations, the inventors also discovered that when the expression of c-MYC or MYCN was enforced in three different genetically modified cell lines, an increase in the responsiveness to an NMT inhibitor was observed, as evidenced by the lower effective concentrations of NMT inhibitor needed to impart cell death in cell lines with high MYC expression compared to those with low MYC expression, and also the shorter timeframes for cell death—see, for instance, FIGS. 4, 5, 14 and 15.

The oncogenes c-MYC and MYCN are common diagnostic markers for particularly aggressive types of malignancies and there are recent findings demonstrating that c-MYC and MYCN overexpression and/or mutation correlate strongly with some of the worst clinical outcomes (Jung et al., Tumor and Stem Cell Biology, 2016, 65(16), 7065-7070, Habermann et al., Blood, 2016, 128(22), 155, Xu et al., Genes Cancer, 2010, 1(6), 629-640). There therefore remains a need for improved treatments that are able to target cancers where one or more structural alterations of the MYC oncogene exist.

In line with the advantageous properties described hereinabove, the inventors have therefore discovered that the class of NMT inhibitor compounds, particularly the NMT inhibitor compounds defined herein, are especially well-suited for application in the treatment of cancers which: i) are addicted to the MYC oncogene; and/or ii) have one or more structural alterations in the MYC oncogene locus.

Thus, as mentioned above, the present invention provides a NMT inhibitor, or a pharmaceutically acceptable salt, solvate or hydrate thereof, for use in the treatment of a MYC addicted cancer. Suitably, the MYC addicted cancer is a cancer which is addicted to c-MYC and/or MYCN. In certain embodiments, the MYC addicted cancer is a cancer which is addicted to c-MYC, and, suitably, the MYC addicted cancer is a cancer which is transcriptionally addicted to c-MYC. In other embodiments, the MYC addicted cancer is a cancer which is addicted to MYCN, and, suitably, the MYC addicted cancer is a cancer which is transcriptionally addicted to MYCN.

In a particular embodiment, the present invention provides a NMT inhibitor, or a pharmaceutically acceptable salt, solvate or hydrate thereof, for use in the treatment of a c-MYC or MYCN addicted cancer, wherein the c-MYC or MYCN oncogene is overexpressed. Suitably, the present invention provides a NMT inhibitor, or a pharmaceutically acceptable salt, solvate or hydrate thereof, for use in the treatment of a c-MYC or MYCN addicted cancer, wherein the c-MYC or MYCN oncogene is overexpressed such that the levels of RNA transcript and/or protein of c-MYC or MYCN are at least 25% greater than the RNA transcript and/or protein levels of c-MYC or MYCN found in a normal, healthy cell. More suitably, the present invention provides a NMT inhibitor, or a pharmaceutically acceptable salt, solvate or hydrate thereof, for use in the treatment of a c-MYC or MYCN addicted cancer, wherein the c-MYC or MYCN is overexpressed such that the levels of RNA transcript and/or protein of c-MYC or N-MYC are at least 50% greater than the RNA transcript and/or protein levels of c-MYC or N-MYC found in a normal, healthy cell.

The present invention also provides a NMT inhibitor, or a pharmaceutically acceptable salt, solvate or hydrate thereof, for use in the treatment of a MYC dysregulated cancer.

It will be readily understood that a MYC dysregulated cancer may include, for example, cancers comprising mutations and/or structural alterations of the MYC oncogene which impart, for instance, overexpression or stabilisation of the protein and/or mRNA levels of MYC. A non-limiting list of possible mutations include: i) point mutations in the MYC coding region (Bahram et. al., Blood, 2000, 95, 2104-2110); ii) mutations of distal enhancers (Sur et al., Science, 2012, 338, 1360-1363 and Zhang et al., Nat. Genet., 2016, 48-176-182); and iii) activating mutations in the signal transduction pathways that augment MYC expression (Herranz et al., Nat. Med., 2014, 20, 1130-1137, Muncan et al., Mol. Cell Biol., 2006, 26, 8418-8426 and Weng et al., Genes Dev., 2006, 20, 2096-2109).

In an embodiment, the MYC dysregulated cancer is a MYC oncogene overexpressing cancer. Suitably, the MYC dysregulated cancer is a c-MYC or MYCN oncogene overexpressing cancer. More suitably, the MYC oncogene dysregulated cancer is a c-MYC or MYCN oncogene overexpressing cancer, wherein the c-MYC or MYCN is overexpressed such that the levels of RNA transcript and/or protein of c-MYC or MYCN are at least 25% greater than the RNA transcript and/or protein levels of c-MYC or MYCN found in a normal, healthy cell. Most suitably, the MYC dysregulated cancer is a c-MYC or MYCN oncogene overexpressing cancer, wherein the c-MYC or MYCN is overexpressed such that the levels of RNA transcript and/or protein of c-MYC or MYCN are at least 50% greater than the RNA transcript and/or protein levels of c-MYC or MYCN found in a normal, healthy cell.

The present invention further provides a NMT inhibitor, or a pharmaceutically acceptable salt, solvate or hydrate thereof, for use in the treatment of a cancer, wherein said cancer comprises one or more structural alterations of the MYC locus. Non-limiting examples of “structural alterations” include, for example, mutations, copy-number gains and/or chromosomal rearrangements. In one embodiment, there is provided a NMT inhibitor, or a pharmaceutically acceptable salt, solvate or hydrate thereof, for use in the treatment of a cancer, wherein said cancer comprises one or more mutations of the MYC locus. Suitably, the present invention provides a NMT inhibitor, or a pharmaceutically acceptable salt, solvate or hydrate thereof, for use in the treatment of a cancer, wherein said cancer comprises one or more mutations of the MYC locus which impart overexpression of MYC. More suitably, the present invention provides a NMT inhibitor, or a pharmaceutically acceptable salt, solvate or hydrate thereof, for use in the treatment of a cancer, wherein said cancer comprises one or more mutations of the MYC locus which impart overexpression of c-MYC or MYCN. In one particular embodiment, the present invention provides a NMT inhibitor, or a pharmaceutically acceptable salt, solvate or hydrate thereof, for use in the treatment of a cancer, wherein said cancer comprises one or more mutations of c-MYC. In another particular embodiment, the invention provides a NMT inhibitor, or a pharmaceutically acceptable salt, solvate or hydrate thereof, for use in the treatment of a cancer, wherein said cancer comprises one or more mutations of MYCN.

In a particular embodiment, there is provided a NMT inhibitor, or a pharmaceutically acceptable salt, solvate or hydrate thereof, for use in the treatment of a cancer, wherein said cancer comprises one or more mutations of the MYC locus which impart stabilization of MYC.

In a particular embodiment, the MYC addicted cancer, the MYC dysregulated cancer or the cancer comprising one or more structural alterations of the MYC locus, is a cancer selected from a haematologic malignancy or a solid-tumour.

In another embodiment, the MYC addicted cancer, the MYC dysregulated cancer or the cancer comprising one or more structural alterations of the MYC locus, is a haematological malignancy. Suitably, the MYC addicted cancer, the MYC dysregulated cancer or the cancer comprising one or more structural alterations of the MYC locus, is a haematological malignancy selected from lymphoma, myeloma or leukaemia.

In another embodiment, the MYC addicted cancer, the MYC dysregulated cancer or the cancer comprising one or more structural alterations of the MYC locus, is a lymphoma.

Suitably, the MYC addicted cancer, the MYC dysregulated cancer or the cancer comprising one or more structural alterations of the MYC locus, is a lymphoma selected from high grade mantle zone lymphoma, follicular lymphoma, plasmablastic lymphoma, diffuse large B-cell lymphoma and Burkitt's lymphoma. More suitably, the MYC addicted cancer, the MYC dysregulated cancer or the cancer comprising one or more structural alterations of the MYC locus, is a lymphoma selected from diffuse large B-cell lymphoma or Burkitt's lymphoma. Most suitably, the MYC addicted cancer, the MYC dysregulated cancer or the cancer comprising one or more structural alterations of the MYC locus, is a diffuse large B-cell lymphoma.

In another embodiment, the MYC addicted cancer, the MYC dysregulated cancer or the cancer comprising one or more structural alterations of the MYC locus, is a myeloma. Suitably, the MYC addicted cancer, the MYC dysregulated cancer or the cancer comprising one or more structural alterations of the MYC locus, is a multiple myeloma.

In another embodiment, the MYC addicted cancer, the MYC dysregulated cancer or the cancer comprising one or more structural alterations of the MYC locus, is a leukaemia. Suitably, the MYC addicted cancer, the MYC dysregulated cancer or the cancer comprising one or more structural alterations of the MYC locus, is a leukaemia selected from chronic lymphocytic leukaemia, acute myeloid leukemia and B-acute lymphocytic leukaemia. Most suitably, the MYC addicted cancer, the MYC dysregulated cancer or the cancer comprising one or more structural alterations of the MYC locus, is a B-acute lymphocytic leukaemia.

In another embodiment, the MYC addicted cancer, the MYC dysregulated cancer or the cancer comprising one or more structural alterations of the MYC locus, is a blastoma. Suitably, the MYC addicted cancer, the MYC dysregulated cancer or the cancer comprising one or more structural alterations of the MYC locus, is a blastoma selected from a neuroblastoma, a retinoblastoma and a glioblastoma. More suitably, the MYC addicted cancer, the MYC dysregulated cancer or the cancer comprising one or more structural alterations of the MYC locus, is a blastoma selected from a retinoblastoma and a glioblastoma. In a particular embodiment, the MYC addicted cancer, the MYC dysregulated cancer or the cancer comprising one or more structural alterations of the MYC locus, is a neuroblastoma.

In another embodiment, the MYC addicted cancer, the MYC dysregulated cancer or the cancer comprising one or more structural alterations of the MYC locus, is a neuronal-originating-cancer (i.e. a cancer of the nervous system). In a particular embodiment, the MYC addicted cancer, the MYC dysregulated cancer or the cancer comprising one or more structural alterations of the MYC locus, is selected from a neuroblastoma, retinoblastoma, a glioblastoma, a small cell lung carcinoma and an astrocytoma.

In another embodiment, the MYC addicted cancer, the MYC dysregulated cancer or the cancer comprising one or more structural alterations of the MYC locus, is a solid-tumour.

In certain embodiments, the solid-tumour is a carcinoma. Suitably, the MYC addicted cancer, the MYC dysregulated cancer or the cancer comprising one or more structural alterations of the MYC locus, is a solid tumour in an organ selected from brain, lung, breast, prostate, ovary, colon, gallbladder, kidney and liver. More suitably, the MYC addicted cancer, the MYC dysregulated cancer or the cancer comprising one or more structural alterations of the MYC locus, is a solid tumour in an organ selected from brain, breast, prostate, colon, gallbladder and kidney. Yet more suitably, the MYC addicted cancer, the MYC dysregulated cancer or the cancer comprising one or more structural alterations of the MYC locus, is a solid tumour in an organ selected from breast, colon and gallbladder.

In certain embodiments, the MYC addicted cancer, the MYC dysregulated cancer or the cancer comprising one or more structural alterations of the MYC locus, is a solid tumour selected from ovarian serous cystadenocarcinoma, esophageal carcinoma, lung squamous cell carcinoma, lung adenocarcinoma, bladder urothelial carcinoma, uterine carcinosarcoma, stomach adenocarcinoma, breast invasive carcinoma and liver hepatocellular carcinoma.

In certain embodiments, the MYC addicted cancer, the MYC dysregulated cancer or the cancer comprising one or more structural alterations of the MYC locus, is breast cancer. Suitably, the MYC addicted cancer, the MYC dysregulated cancer or the cancer comprising one or more structural alterations of the MYC locus, is triple negative breast cancer or a breast invasive carcinoma. In particular embodiments, the MYC addicted cancer, the MYC dysregulated cancer or the cancer comprising one or more structural alterations of the MYC locus, is basal-like breast cancer.

In other embodiments, the MYC addicted cancer, the MYC dysregulated cancer or the cancer comprising one or more structural alterations of the MYC locus, is cancer of the gallbladder.

In other embodiments, the MYC addicted cancer, the MYC dysregulated cancer or the cancer comprising one or more structural alterations of the MYC locus, is colorectal cancer.

In other embodiments, the MYC addicted cancer, the MYC dysregulated cancer or the cancer comprising one or more structural alterations of the MYC locus, is brain cancer (e.g. an astrocytoma).

In certain embodiments, the MYC addicted cancer, the MYC dysregulated cancer or the cancer comprising one or more structural alterations of the MYC locus, is selected from diffuse large B-cell lymphoma, a Burkitt's lymphoma, multiple myeloma, blastoma (e.g. a neuroblastoma, retinoblastoma or glioblastoma), acute myeloid leukemia, B-acute lymphocytic leukaemia and a solid tumour in an organ selected from breast, colon and gallbladder. Suitably, the MYC addicted cancer, the MYC dysregulated cancer or the cancer comprising one or more structural alterations of the MYC locus, is selected from diffuse large B-cell lymphoma, Burkitt's lymphoma, neuroblastoma, retinoblastoma, glioblastoma, acute myeloid leukemia, B-acute lymphocytic leukaemia and breast cancer. More suitably, the MYC addicted cancer, the MYC dysregulated cancer or the cancer comprising one or more structural alterations of the MYC locus, is selected from diffuse large B-cell lymphoma, neuroblastoma, B-acute lymphocytic leukaemia and triple negative breast cancer.

In particular embodiments, the MYC addicted cancer, the MYC dysregulated cancer or the cancer comprising one or more structural alterations of the MYC locus, is colorectal cancer, gallbladder carcinoma, brain tumour, lymphoma (such as diffuse large B-cell lymphoma), leukemia (such as acute myeloid leukemia) or blastoma (such as neuroblastoma, retinoblastoma or glioblastoma).

In certain embodiments, the MYC addicted cancer, the MYC dysregulated cancer or the cancer comprising one or more structural alterations of the MYC locus, is diffuse large B-cell lymphoma, Burkitt's lymphoma, multiple myeloma, blastoma (such as neuroblastoma, retinoblastoma or glioblastoma), acute myeloid leukemia, B-acute lymphocytic leukaemia or triple negative breast cancer. Suitably, the MYC addicted cancer, the MYC dysregulated cancer or the cancer comprising one or more structural alterations of the MYC locus, is multiple myeloma, neuroblastoma, retinoblastoma, glioblastoma, acute myeloid leukemia, B-acute lymphocytic leukaemia or triple negative breast cancer. More suitably, the MYC addicted cancer, the MYC dysregulated cancer or the cancer comprising one or more structural alterations of the MYC locus, is multiple myeloma, neuroblastoma, retinoblastoma, glioblastoma, or triple negative breast cancer. Even more suitably, the MYC addicted cancer, the MYC dysregulated cancer or the cancer comprising one or more structural alterations of the MYC locus, is neuroblastoma or triple negative breast cancer (e.g. basal-like breast cancer).

In a particular embodiment, the invention provides a NMT inhibitor, or a pharmaceutically acceptable salt, solvate or hydrate thereof, for use in the treatment of a blastoma, wherein said blastoma comprises one or more mutations of MYCN. Suitably, the blastoma is selected from a neuroblastoma, a retinoblastoma and a glioblastoma. Most suitably, the invention provides a NMT inhibitor, or a pharmaceutically acceptable salt, solvate or hydrate thereof, for use in the treatment of a neuroblastoma, wherein said neuroblastoma comprises one or more mutations of MYCN.

In another particular embodiment, the invention provides a NMT inhibitor, or a pharmaceutically acceptable salt, solvate or hydrate thereof, for use in the treatment of a c-MYC addicted cancer, wherein the c-MYC addicted cancer is a breast cancer. Suitably, the invention provides a NMT inhibitor, or a pharmaceutically acceptable salt, solvate or hydrate thereof, for use in the treatment of a c-MYC addicted cancer, wherein the c-MYC addicted cancer is a triple negative breast cancer (e.g. a basal-like breast cancer) or a breast invasive carcinoma.

The present invention also provides a method for the treatment of a MYC addicted cancer in a subject in need of such treatment, said method comprising administering a therapeutically effective amount of a NMT inhibitor, or a pharmaceutically acceptable salt, solvate or hydrate thereof. Suitably, the MYC addicted cancer is any one of the MYC addicted cancers described hereinabove.

Further provided is a method for the treatment of a MYC dysregulated cancer in a subject in need of such treatment, said method comprising administering a therapeutically effective amount of a NMT inhibitor, or a pharmaceutically acceptable salt, solvate or hydrate thereof. Suitably, the MYC dysregulated cancer is any one of the MYC dysregulated cancers described hereinabove.

Further provided is a method for the treatment of a cancer comprising one or more structural alterations of the MYC locus in a subject in need of such treatment, said method comprising administering a therapeutically effective amount of a NMT inhibitor, or a pharmaceutically acceptable salt, solvate or hydrate thereof. Suitably, the cancer comprising one or more structural alterations of the MYC locus is any one of the cancers described hereinabove.

According to a further aspect of the present invention, there is provided a method for determining whether a subject with a cancer will benefit from treatment with an NMT inhibitor, said method comprising the steps of:
i) taking a sample of cancer cells taken from said subject;
ii) analysing the cancer cells of step i) to check for the presence of one or more structural alterations (e.g. chromosomal rearrangements, copy number gains and/or mutations) in the MYC locus;
iii) determining whether one or more structural alterations (e.g. chromosomal rearrangements, copy number gains and/or mutations) are present in the MYC locus of the sample of cancer cells when compared to a control; and
iv) determining whether the subject will benefit from being administered a NMT inhibitor in order to treat said cancer, wherein if the sample of cancer cells contains one or more structural alterations (e.g. chromosomal rearrangements, copy number gains and/or mutations) in the MYC locus, then the subject will benefit from being administered a NMT inhibitor, and if the sample of cancer cells does not contain one or more structural alterations (e.g. chromosomal rearrangements, copy number gains and/or mutations) in the MYC locus, then the subject will not benefit from being administered a NMT inhibitor.

In an embodiment, the one or more structural alterations are chromosomal rearrangements. Thus, suitably, step ii) of the above method involves analysing the cancer cells of step i) to check for the presence of one or more chromosomal rearrangements in the MYC locus. The person skilled in the art will be able to readily determine suitable techniques for checking for the presence of one or more chromosomal rearrangements in the MYC locus. One, non-limiting, example of a suitable technique for checking for the presence of one or more chromosomal rearrangements in the MYC locus is fluorescence in-situ hybridisation (FISH).

In another embodiment, the one or more structural alterations are mutations. Thus, suitably, step ii) of the above method involves analysing the cancer cells of step i) to check for the presence of one or more mutations in the MYC locus. The person skilled in the art will be able to readily determine suitable techniques for checking for the presence of one or more mutations in the MYC locus. One, non-limiting, example of a suitable technique for checking for the presence of one or more mutations in the MYC locus is gene sequencing.

Suitably, the control of step iii) is the structural arrangement of the MYC locus found in a normal, healthy cell, specifically, the control of step iii) is the chromosomal arrangement and/or genetic sequence found in the MYC locus of a normal, healthy cell.

It will be understood that the sample of cancer cells taken from said subject may be obtained by any suitable method known in the art. For instance, the sample of cancer cells taken from said subject may be those taken from a biopsy or may be a sample of circulating tumour cells (CTCs) taken from the subject.

According to a further aspect of the present invention, there is provided a method for determining whether a subject with a cancer will benefit from treatment with an NMT inhibitor, said method comprising the steps of:
i) measuring the level of MYC expression in a sample of cancer cells taken from said subject;
ii) comparing the level of MYC expression from step i) with a control;
iii) determining whether the MYC expression in the sample of cancer cells is increased compared to the control; and
iv) determining whether the subject will benefit from being administered a NMT inhibitor in order to treat said cancer, wherein if the MYC expression in the sample of cancer cells is higher than in the control, then the subject will benefit from being administered a NMT inhibitor, and if the MYC expression in the sample of cancer cells is not higher than in the control, then the subject will not benefit from being administered a NMT inhibitor.

It will be appreciated that the level of MYC expression in the sample of cancer cells may be determined by any suitable means known in the art. For example, the level of expression of MYC may be determined by measuring MYC protein levels. The MYC protein levels may be measured using any suitable technique known in the art, such as, for example, SDS-PAGE followed by Western blot using suitable antibodies raised against the target protein. In addition, or alternatively, the level of expression of MYC may be determined by measuring the level of mRNA. The level of mRNA may be measured using any suitable technique known in the art, such as, for example, northern blot or quantitative RT-PCR (qRT-PCR).

Suitably, the control of step ii) above is the MYC expression level found in a normal, healthy cell, for example, a normal, healthy cell of the same type as the cell being investigated. It will be understood that the MYC expression level found in a normal, healthy cell may also be determined using any of the techniques described in paragraph [0091] above.

According to a further aspect of the present invention, there is provided a method for the treatment of cancer in a subject who has been identified as benefiting from being administered a NMT inhibitor as determined by a method as described in any one of paragraphs [0085] to [0090] above, wherein said method comprises administering a therapeutically effective amount of a NMT inhibitor, or a pharmaceutically acceptable salt, solvate or hydrate thereof to the subject. Suitably, the NMT inhibitor may be any of the NMT inhibitors described hereinbelow.

### Routes of Administration

The NMT inhibitors for use in the present invention may be administered to a subject by any convenient route of administration, whether systemically/peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly. In certain embodiments, the NMT inhibitors for use in the present invention are administered to a subject by oral administration (e.g, by ingestion).

The amount of NMT inhibitor which is required to achieve a therapeutic effect will, of course, vary with the particular compound, the route of administration, the subject under treatment, including the type, species, age, weight, sex, and medical condition of the subject and the renal and hepatic function of the subject, and the particular disorder or disease being treated, as well as its severity. An ordinarily skilled physician, veterinarian or clinician can readily determine and prescribe the effective amount of the drug required to prevent, counter or arrest the progress of the condition.

An effective amount of the NMT inhibitor for use in the treatment of cancer as described herein is an amount sufficient to treat or prevent a cancer mentioned herein, slow its progression and/or reduce the symptoms associated with the cancer.

When orally administered to humans, the NMT inhibitor for use in the present invention will generally be administered at an amount of between about 0.01 mg per kg of body weight per day (mg/kg/day) to about 100 mg/kg/day, preferably 0.01 mg per kg of body weight per day (mg/kg/day) to 10 mg/kg/day, and most preferably 0.1 to 5.0 mg/kg/day, for adult humans.

The size of the dose for therapeutic or prophylactic purposes of a compound of the formula I will naturally vary according to the nature and severity of the conditions, the age and sex of the animal or patient and the route of administration, according to well-known principles of medicine.

In using a NMT inhibitor for the treatment of cancer it will generally be administered so that a daily dose in the range, for example, 0.1 mg/kg to 75 mg/kg body weight is received, given if required in divided doses. In general, lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a NMT inhibitor for use in this invention.

### Combination Therapies

The NMT inhibitor defined hereinbefore may be applied as a sole therapy or be administered in combination with one or more other therapeutic agents, or may be administered in combination with conventional surgery or radiotherapy.

Such conjoint treatment may be achieved by way of the simultaneous, sequential or separate dosing of the NMT inhibitor and the one or more other therapeutic agents of the treatment. Such combination products may employ the NMT inhibitors of this invention within any suitable dosage range, such as, for example, the dosage range described hereinabove, and the other pharmaceutically-active agent may be within its approved dosage range.

Thus, the present invention further provides a NMT inhibitor, or a pharmaceutically acceptable salt, solvate or hydrate thereof, for use in combination with one or more other therapeutic agents in the treatment of a MYC addicted cancer. Suitably, the MYC addicted cancer is any one of the MYC addicted cancers described hereinabove.

The present invention also provides a NMT inhibitor, or a pharmaceutically acceptable salt, solvate or hydrate thereof, for use in combination with one or more other therapeutic agents in the treatment of a MYC dysregulated cancer. Suitably, the MYC dysregulated cancer is any one of the MYC dysregulated cancers described hereinabove.

The present invention also provides a NMT inhibitor, or a pharmaceutically acceptable salt, solvate or hydrate thereof, for use in combination with one or more other therapeutic agents in the treatment of a cancer comprising one or more structural alterations of the MYC locus. Suitably, the cancer comprising one or more structural alterations of the MYC locus is any one of the cancers comprising one or more structural alterations of the MYC locus described hereinabove.

Suitable, but non-limiting, examples of other therapeutic agents which may be administered in combination with the NMT inhibitor include one or more other chemotherapeutic agents.

For example, the NMT inhibitor may be administered in addition to any currently recommended treatment (also known as the “standard of care”) for cancer.

### N-Myristoyl Transferase (NMT) Inhibitors

N-myristoyl transferase (NMT) is a monomeric enzyme, which is ubiquitous in eukaryotes. NMT catalyses an irreversible co-translational transfer of myristic acid (a saturated 14-carbon fatty acid) from myristoyl-Coenzyme A (myr-CoA) to a protein substrate containing an N-terminal glycine with formation of an amide bond (Farazi, T. A., G. Waksman, and J. I. Gordon, J. Biol. Chem., 2001. 276(43): p. 39501-39504).

There are two types of human NMT, human NMT1 (HsNMT1) and human NMT2 (HsNMT2). Inhibition of human NMT has been suggested as a target for treating or preventing various diseases or disorders, for example hyperproliferative disorders (cancers, e.g. human colorectal cancer, gallbladder carcinoma, brain tumours, and lymphomas such as B-cell lymphoma) (Resh M D. 1993. Biochem. Biophys. Acta 1115, 307-22; Berthiaume L G, Beauchamp E, WO2017/011907). As NMT plays a key role in protein trafficking, mediation of protein-protein interactions, stabilization of protein structures and signal transduction in living systems, inhibition of the NMT enzyme has the potential to disrupt multi-protein pathways. This is an attractive characteristic to reduce the risk of the development of resistance in, for example, treatment or prevention of hyperproliferative disorders.

Compounds active as inhibitors of NMT have previously been disclosed, see for example WO00/37464 (Roche), WO2010/026365 (University of Dundee), WO2013/083991 (Imperial Innovations Limited) and WO2017/001812 (Imperial Innovations Limited), and their use in the treatment of cancer has been described.

Thus, the NMT inhibitors for use in the present invention are species (e.g. compounds) which display activity as inhibitors of N-myristoyl transferase (NMT). Furthermore, the term “NMT inhibitor” as used herein will be understood to cover any species which binds to NMT and inhibits its activity. The inhibitors may act as competitive inhibitors, or partial competitive inhibitors. The inhibitor may bind to NMT at the myr-CoA binding pocket or at the peptide binding pocket (or inhibit NMT through another mechanism). The NMT inhibitor of the present invention preferably bind and inhibit NMT through the peptide binding pocket.

Suitably, the NMT inhibitors for use in the present invention are compounds which display activity as inhibitors of N-myristoyl transferase (NMT). Suitable compounds which display activity as inhibitors of NMT are known in the art. Non-limiting examples of suitable NMT inhibitors are described in, for example, WO00/37464 (Roche), WO2010/026365 (University of Dundee), WO2013/083991 (Imperial Innovations Limited) and WO2017/001812 (Imperial Innovations Limited), the entire contents of which are incorporated herein by reference.

In a particular embodiment, the NMT inhibitor for use in the present invention is a compound of Formula I, Formula II or Formula III, as defined herein, or a pharmaceutically acceptable salt, solvate or hydrate thereof. For the avoidance of doubt, this embodiment of the present invention also encompasses all sub-formulae of Formula I, Formula II and Formula III described herein, such as for example, compounds of Formula (IA\hat{ }\hat{ }) and/or Formula IIa.

In certain embodiments, the NMT inhibitor for use in the present invention is a compound of Formula I or Formula II, or a pharmaceutically acceptable salt, solvate or hydrate thereof. In other embodiments, the NMT inhibitor for use in the present invention is a compound of Formula I or Formula III, or a pharmaceutically acceptable salt, solvate or hydrate thereof. In yet further embodiments, the NMT inhibitor for use in the present invention is a compound of Formula II or Formula III, or a pharmaceutically acceptable salt, solvate or hydrate thereof.

In particular embodiments, the NMT inhibitor for use in the present invention is a compound of Formula I or a pharmaceutically acceptable salt, solvate or hydrate thereof. Suitably, the NMT inhibitor for use in the present invention is a compound of Formula (IA\hat{ }\hat{ }) or a pharmaceutically acceptable salt, solvate or hydrate thereof.

In other embodiments, the NMT inhibitor for use in the present invention is a compound of Formula II, or a pharmaceutically acceptable salt, solvate or hydrate thereof. Suitably, the NMT inhibitor of the present invention is a compound of Formula IIa, or a pharmaceutically acceptable salt, solvate or hydrate thereof.

### NMT Inhibitors of Formula (I)

As outlined above, in certain embodiments, the NMT inhibitor is a compound of Formula (I) shown below, or a pharmaceutically acceptable salt, hydrate or solvate thereof:

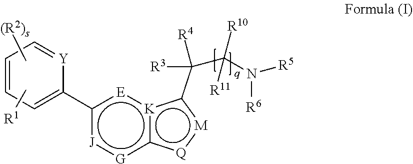

wherein:
Y is selected from the group consisting of —CH—, —C(R²)— and —N—;
R¹ is a group of formula —X-L-A;
wherein:
   X is selected from the group consisting of —O—, —N(H)— and —S—, or is absent;
   L is selected from the group consisting of —(CHR¹²)ₘ— and —(CHR¹²)ₘO—, or is absent;
   m is 1, 2 or 3; and
   A is a 6-10-membered aromatic carbocycle or a 5-10-membered aromatic heterocycle, said aromatic carbocycle or heterocycle being optionally substituted with 1, 2, or 3 substituents each independently selected from the group consisting of —F, —Cl, —Br, —OCH₃, —OCF₃, —CN, —C₁₋₆alkyl optionally substituted by up to 3 halogen, hydroxyl, or —OC₁₋₄alkyl groups, —S(O)C₁₋₄alkyl, —S(O)₂C₁₋₄alkyl, —C(O)N(R⁹)₂, —C(O)N(R¹³)C₁₋₄alkylOC₁₋₄alkyl, —C(O)N(C₁₋₄alkylOC₁₋₄alkyl)₂, —CH—₂C(O)N(R⁹)₂, —CH₂C(O)N(R¹³)C₁₋₄alkylOC₁₋₄alkyl, —CH₂C(O)N(C₁₋₄alkylOC₁₋₄alkyl)₂, —S(O)₂NHC₁₋₄alkyl, —S(O)₂N(C₁₋₄alkyl)₂, —NHC₁₋₄alkyl, —N(C₁₋₄alkyl)₂, —NHC(O)C₁₋₄alkyl, —NHC(O)CF₃, —NHS(O)₂C₁₋₄alkyl, CH₂N(R¹³)₂, CH₂N(R¹³)C(O)C₁₋₄alkyl, CH₂N(R¹³)S(O)₂C₁₋₄alkyl, —CH₂S(O)₂C₁₋₄alkyl, and CO₂H;
s is 0, 1, 2, or 3;
each R² is independently selected from the group consisting of —F, —Cl, —Br, —OCH₃, —OCF₃, —CN, —C₁₋₄alkyl optionally substituted by up to 3 halogen or hydroxyl groups, —S(O)C₁₋₄alkyl, —S(O)₂C₁₋₄alkyl, —S(O)₂NHC₁₋₄alkyl, —S(O)₂N(C₁₋₄alkyl)₂, —NHC₁₋₄alkyl, —N(C₁₋₄alkyl)₂, —NHC(O)C₁₋₄alkyl, —NHC(O)CF₃, and —NHS(O)₂C₁₋₄alkyl;
E, J and G are each independently nitrogen or C(R⁷);
K is carbon or nitrogen;
and wherein:
i) when K is carbon, either Q is N(R⁸) and M is nitrogen or C(R⁷), or Q is nitrogen and M is N(R⁸); or
ii) when K is nitrogen, Q is nitrogen or C(R⁷) and M is nitrogen or C(R⁷); and further wherein at least 2 of E, J, G, K, Q and M are selected from the group consisting of carbon and C(R⁷);
q is 0 or 1;
R³ is hydrogen or methyl; R⁴ is hydrogen or methyl;
R⁵ is hydrogen or C₁₋₆alkyl optionally substituted by up to 3 —F, —Cl, —Br, —OH, —OCH₃, —OCF₃ or —CN groups;
R⁶ is hydrogen or C₁₋₆alkyl optionally substituted by up to 3 —F, —Cl, —Br, —OH, —OCH₃, —OCF₃ or —CN groups;
or the R⁵ and R⁶ groups and the N they are bonded to form a 4 to 7 membered non-aromatic heterocycle, the heterocycle optionally comprising 1 or 2 further heteroatoms selected from N, O and S, optionally substituted by up to 3 —F, —Cl, —Br, —OH, —OCH₃, —OCF₃ or —CN groups;
when present R¹⁰ is hydrogen or methyl;
when present R¹¹ is hydrogen or methyl;
or the R³ group and the R⁵ group and the intervening atoms form a 3 to 7 membered non-aromatic heterocycle composed of the intervening atoms and bond, or the intervening atoms and —(CHR^{a})ᵣ—;
or the R¹⁰ group and the R⁵ group and the intervening atoms form a 3 to 7 membered non-aromatic heterocycle composed of the intervening atoms and —(CHR^{a})ᵣ—;
r is 1, 2, 3, 4 or 5; R^{a} is hydrogen or methyl;
each R⁷ is independently selected from the group consisting of hydrogen, halogen, C₁₋₄alkoxy, and C₁₋₄alkyl optionally substituted with 1, 2 or 3 halogens; and
R⁸ is selected from the group selected from hydrogen and C₁₋₄alkyl;
each R⁹ is independently selected from the group consisting of hydrogen and C₁₋₄alkyl, or two R⁹ groups and the N they are bonded to form a 4 to 7 membered non-aromatic heterocycle, the heterocycle optionally comprising 1 or 2 further heteroatoms selected from N, O and S;
each R¹² is independently selected from the group consisting of hydrogen, C₁₋₆alkyl optionally substituted by up to 3 —F, —Cl, —Br, I, —OH, —OCH₃, —OCF₃ or —CN groups, C₁₋₆alkenyl optionally substituted by up to 3 —F, —Cl, —Br, I, —OH, —OCH₃, —OCF₃ or —CN groups, and C₁₋₆alkynyl optionally substituted by up to 3 —F, —Cl, —Br, I, —OH, —OCH₃, —OCF₃ or —CN groups; and each R¹³ is independently selected from the group consisting of hydrogen and C₁₋₄alkyl

NMT inhibitors of Formula (I) are further described in WO2017/001812. It will be understood that suitable and preferred NMT inhibitors of Formula (I) of the present invention may include any of the compounds (generic or specific) disclosed in WO2017/001812.

In one embodiment, X is selected from the group consisting of —O—, —N(H)— and —S—. In another embodiment, L is selected from the group consisting of —(CHR¹²)ₘ— and —(CHR¹²)ₘO—. Suitably, X is selected from the group consisting of —O—, —N(H)— and —S—, and L is selected from the group consisting of —(CHR¹²)ₘ— and —(CHR¹²)ₘO—.

In certain embodiments, E, J, G and M are each C(R⁷), and K and Q are each nitrogen. In other embodiments, q is 1, R¹⁰ is hydrogen and R¹¹ is hydrogen. Suitably, q is 1, R¹⁰ is hydrogen, R¹¹ is hydrogen, E, J, G and M are each C(R⁷), and K and Q are each nitrogen.

In another embodiment, A is a 6-10-membered aromatic carbocycle or a 5-10-membered aromatic heterocycle, said aromatic carbocycle or heterocycle being optionally substituted with 1, 2, or 3 substituents each independently selected from the group consisting of —F, —Cl, —Br, —OCH₃, —OCF₃, —CN, —C₁₋₆alkyl optionally substituted by up to 3 halogen, hydroxyl, or —OC₁₋₄alkyl groups, —S(O)C₁₋₄alkyl, —S(O)₂C₁₋₄alkyl, —C(O)N(R⁹)₂, —CH₂C(O)N(R⁹)₂, —S(O)₂NHC₁₋₄alkyl, —S(O)₂N(C₁₋₄alkyl)₂, —NHC₁₋₄alkyl, —N(C₁₋₄alkyl)₂, —NHC(O)C₁₋₄alkyl, —CH₂NHC(O)C₁₋₄alkyl, —NHC(O)CF₃ and —NHS(O)₂C₁₋₄alkyl.

In yet another embodiment, A is a 6-10-membered aromatic carbocycle or a 5-10-membered aromatic heterocycle, said aromatic carbocycle or heterocycle being optionally substituted with 1, 2, or 3 substituents each independently selected from the group consisting of —F, —Cl, —Br, —OCH₃, —OCF₃, —CN, —C₁₋₆alkyl optionally substituted by up to 3 halogen, hydroxyl, or —OC₁₋₄alkyl groups, —S(O)C₁₋₄alkyl, —S(O)₂C₁₋₄alkyl, —C(O)N(R⁹)₂, —CH₂C(O)N(R⁹)₂, —S(O)₂NHC₁₋₄alkyl, —S(O)₂N(C₁₋₄alkyl)₂, —NHC₁₋₄alkyl, —N(C₁₋₄alkyl)₂, —NHC(O)C₁₋₄alkyl, —CH₂NHC(O)C₁₋₄alkyl, —NHC(O)CF₃, —NHS(O)₂C₁₋₄alkyl, CH₂NH₂, CH₂NHC₁₋₄alkyl, CH₂NC₁₋₄alkylC(O)C₁₋₄alkyl, CH₂NHS(O)₂C₁₋₄alkyl, —CH₂S(O)₂C₁₋₄alkyl, CH₂NC₁₋₄alkylS(O)₂C₁₋₄alkyl.

In certain embodiments, the NMT inhibitor has the formula (IA), such as, for example, Formula (Iα), or the NMT inhibitor has the formula IB, such as, for example, Formula Iβ, shown below:

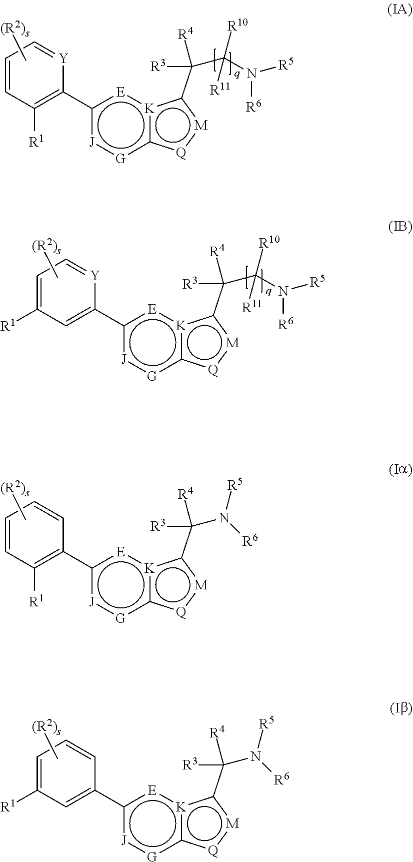

wherein each of R¹, R², s, q, E, J G, K, Q, M, q, R³, R⁴, R⁵ and R⁶, R⁷, R⁸, R⁹, R¹⁰ and R¹¹ are as defined in Formula (I).

In one particular embodiment, the NMT inhibitor has the formula (IA*) shown below:

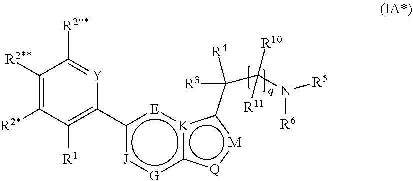

wherein:
each R^{2*} is independently selected from the group consisting of —F, —Cl, —Br, —OCH₃, —OCF₃, —CN, —C₁₋₄alkyl optionally substituted by up to 3 halogen or hydroxyl groups, —S(O)C₁₋₄alkyl, —S(O)₂C₁₋₄alkyl, —S(O)₂NHC₁₋₄alkyl, —S(O)₂N(C₁₋₄alkyl)₂, —NHC₁₋₄alkyl, —N(C₁₋₄alkyl)₂, —NHC(O)C₁₋₄alkyl, —NHC(O)CF₃, and —NHS(O)₂C₁₋₄alkyl (preferably selected from the group consisting of —F, —Cl, —Br, —OCH₃, —OCF₃, —CN, —C₁₋₄alkyl optionally substituted by up to 3 halogen or hydroxyl groups; more preferably selected from the group consisting of —F and —Cl; most preferably —F);
   R^{2**} is selected from the group consisting of —FI, —F, —Cl, —Br, —OCH₃, —OCF₃, —CN, —C₁₋₄alkyl optionally substituted by up to 3 halogen or hydroxyl groups, —S(O)C₁₋₄alkyl, —S(O)₂C₁₋₄alkyl, —S(O)₂NHC₁₋₄alkyl, —S(O)₂N(C₁₋₄alkyl)₂, —NHC₁₋₄alkyl, —N(C₁₋₄alkyl)₂, —NHC(O)C₁₋₄alkyl, —NHC(O)CF₃, and —NHS(O)₂C₁₋₄alkyl (preferably selected from the group consisting of —H, —F, —Cl, —Br, —OCH₃, —OCF₃, —CN, —C₁₋₄alkyl optionally substituted by up to 3 halogen or hydroxyl groups; more preferably selected from the group consisting of —H, —F and —Cl); and
wherein each of R¹, q, E, J, G, K, Q, M, R³, R⁴, R⁵, R⁶, R⁷, R⁸, R⁹, R¹⁰ and R¹¹ are as defined in Formula (I).

Suitably, the NMT inhibitor is a compound of formula (IA***) shown below:

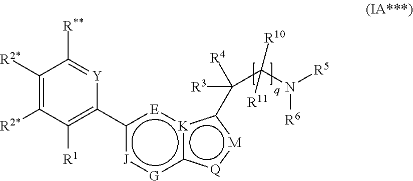

wherein each of R¹, R^{2*}, R^{2**}, q, E, J, G, K, Q, M, R³, R⁴, R⁵, R⁶, R⁷, R⁸, R⁹, R¹⁰ and R¹¹ are as defined hereinabove.

In a particular embodiment, the NMT inhibitor of the invention is a compound of Formula (IA**) or (IB) shown below:

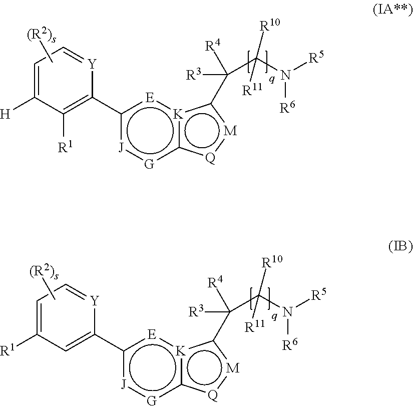

wherein s is 0, 1 or 2 for Formula (IA**); and s is 0, 1, 2 or 3 for Formula (IB); and wherein R¹, R², q, E, J, G, K, Q, M, R³, R⁴, R⁵, R⁶, R⁷, R⁸, R⁹, R¹⁰ and R¹¹ are each as defined in Formula (I).

Suitably, at least two of the variable ring atoms in the core bicyclic moiety of Formula (I), or any one of sub-formulae IA, Iα, IB, Iβ, IA*, IA** and IA***, are carbon. In other words, at least two of E, J, G, K, Q and M are selected from the group consisting of C(R⁷) and carbon (with it being possible for E, J, G, Q and M to be C(R⁷), and it being possible for K to be carbon).

Suitably, at least one of the variable ring atoms in the core bicyclic moiety of Formula (I), or any one of sub-formulae IA, Iα, IB, Iβ, IA*, IA** and IA***, is nitrogen. In other words at least one of E, J, G, K, Q and M is selected from the group consisting of nitrogen and N(R⁸). By way of explanation, K is either carbon or nitrogen and, where K is carbon, either Q is N(R⁸) and M is nitrogen or C(R⁷), or Q is nitrogen and M is N(R⁸).

In one preferred embodiment of the NMT inhibitor of the invention, E, J and G are each C(R⁷), K is carbon, Q is N(R⁸), and M is nitrogen.

In one preferred embodiment of the NMT inhibitor of the invention, E, J and G are each C(R⁷), and K, Q and M are each nitrogen.

In one preferred embodiment of the NMT inhibitor of the invention, E and G are each C(R⁷), and J, K, Q and M are each nitrogen.

In one preferred embodiment of the NMT inhibitor of the invention, J and G are each C(R⁷), and E, K, Q and M are each nitrogen.

In one preferred embodiment of the NMT inhibitor of the invention, E, J, G and M are each C(R⁷), and K and Q are each nitrogen.

More preferably, E, J and G are each C(R⁷), K is carbon, Q is N(R⁸), and M is nitrogen; E, J and G are each C(R⁷), and K, Q and M are each nitrogen; or E, J, G and M are each C(R⁷), and K and Q are each nitrogen. Most preferably E, J and G are each C(R⁷), K is carbon, Q is N(R⁸), and M is nitrogen; or E, J and G are each C(R⁷), and K, Q and M are each nitrogen.

In certain embodiments Y is —CH— or —C(R^{2′})—; preferably Y is —CH—. In another embodiment Y is —N—.

In one preferred embodiment, s is 0, 1 or 2, and, where present, each R² is independently selected from the group consisting of —F, —Cl, —Br, —OCH₃, —OCF₃, —CN, and —C₁₋₄alkyl optionally substituted by up to 3 halogen or hydroxyl groups. More preferably R² is F or Cl.

In one preferred embodiment, A is an aromatic carbocycle or heterocycle selected from the group consisting of phenyl, pyridinyl, quinolinyl, imidazolyl, benzimidazolyl, pyrazolyl, thiazolyl, 1,2,3-triazolyl and 1,2,4-triazolyl, said aromatic carbocycle or heterocycle being optionally substituted with 1, 2, or 3 groups independently selected from the group consisting of —C₁₋₄alkyl (for example methyl), wherein each —C₁₋₄alkyl is optionally substituted by up to 3 halogen, hydroxyl or —OC₁₋₄alkyl groups; C(O)N(R⁹)₂ (for example —C(O)N(H)C₁₋₄alkyl or —C(O)N(R⁹)₂ wherein the two R⁹ groups and the N they are bonded to form a 4 to 7 membered non-aromatic heterocycle, the heterocycle optionally comprising 1 or 2 further heteroatoms selected from N, O and S (for example wherein the two R⁹ and the N they are bonded to form a morpholine or pyrrolidine ring)), —CH₂C(O)N(R⁹)₂ (for example —CH₂C(O)N(H)C₁₋₄alkyl), —CH—₂C(O)N(R⁹)₂ (for example —CH₂C(O)N(H)C₁₋₄alkyl), —C(O)N(R¹³)C₁₋₄alkylOC₁₋₄alkyl (for example —C(O)N(H)C₁₋₄alkylOC₁₋₄alkyl), —N(R⁹)C(O)C₁₋₄alkyl, —CH₂N(R¹³)₂, CH₂N(R⁹)C(O)C₁₋₄alkyl or CH₂N(R¹³)S(O)₂C₁₋₄alkyl; and more preferably C(O)N(R⁹)₂ (for example —C(O)N(H)C₁₋₄alkyl), or —CH₂C(O)N(R⁹)₂ (for example —CH₂C(O)N(H)C₁₋₄alkyl). Preferably A is selected from the group consisting optionally substituted pyrazolyl and thiazolyl.

In one preferred embodiment, A is optionally substituted with 1, 2, or 3 groups independently selected from the group consisting of —C₁₋₄alkyl (for example methyl), wherein each —C₁₋₄alkyl is optionally substituted by up to 3 halogen, hydroxyl or —OC₁₋₄alkyl groups (preferably one —OC₁₋₄alkyl group; more preferably one —OCH₃ group); C(O)N(R⁹)₂ (for example —C(O)N(H)C₁₋₄alkyl or —C(O)N(R⁹)₂ wherein the two R⁹ groups and the N they are bonded to form a 4 to 7 membered non-aromatic heterocycle, the heterocycle optionally comprising 1 or 2 further heteroatoms selected from N, O and S (for example wherein the two R⁹ and the N they are bonded to form a morpholine or pyrrolidine ring)), —CH₂C(O)N(R⁹)₂ (for example —CH₂C(O)N(H)C₁₋₄alkyl), —CH₂C(O)N(R⁹)₂ (for example —CH₂C(O)N(H)C₁₋₄alkyl), —C(O)N(R¹³)C₁₋₄alkylOC₁₋₄alkyl (for example —C(O)N(H)C₁₋₄alkylOC₁₋₄alkyl), —N(R⁹)C(O)C₁₋₄alkyl, —CH₂N(R¹³)₂, CH₂N(R⁹)C(O)C₁₋₄alky or CH₂N(R¹³)S(O)₂C₁₋₄alkyl I; preferably C(O)N(R⁹)₂ (for example —C(O)N(H)C₁₋₄alkyl), —CH₂C(O)N(R⁹)₂ (for example —CH₂C(O)N(H)C₁₋₄alkyl). Preferably A is selected from the group consisting optionally substituted pyrazolyl and thiazolyl.

In certain preferred embodiments, A is optionally substituted with 1, 2, or 3 groups independently selected from the group consisting of —C₁₋₄alkyl (for example methyl), wherein each —C₁₋₄alkyl is optionally substituted by up to 3 halogen, hydroxyl or —OC₁₋₄alkyl groups (preferably one —OC₁₋₄alkyl group; more preferably one —OCH₃ group); —C(O)N(H)C₁₋₄alkyl (for example —C(O)N(H)CH₃) or —C(O)N(R⁹)₂ wherein the two R⁹ groups and the N they are bonded to form a 4 to 7 membered non-aromatic heterocycle, the heterocycle optionally comprising 1 or 2 further heteroatoms selected from N, O and S (for example wherein the two R⁹ and the N they are bonded to form a morpholine or pyrrolidine ring), —CH₂C(O)N(H)C₁₋₄alkyl, —CH—₂C(O)N(H)C₁₋₄alkyl, —C(O)N(H)C₁₋₄alkylOC₁₋₄alkyl, —N(R⁹)C(O)C₁₋₄alky, —CH₂N(R¹³)₂, CH₂N(R⁹)C(O)C₁₋₄alkyl or CH₂N(R¹³)S(O)₂C₁₋₄alkyl. Preferably A is selected from the group consisting optionally substituted pyrazolyl and thiazolyl.

In even more preferred embodiments, A is optionally substituted with 1, 2, or 3 groups independently selected from the group consisting of —C₁₋₄alkyl (for example methyl), wherein each —C₁₋₄alkyl is optionally substituted by up to 3 halogen, hydroxyl or —OC₁₋₄alkyl groups (preferably one —OC₁₋₄alkyl group; more preferably one —OCH₃ group); —C(O)N(H)C₁₋₄alkyl (for example —C(O)N(H)CH₃) or —C(O)N(R⁹)₂ wherein the two R⁹ and the N they are bonded to form a morpholine or pyrrolidine ring (preferably a morpholine ring), —CH₂C(O)N(H)C₁₋₄alkyl, —CH—₂C(O)N(H)C₁₋₄alkyl, and —C(O)N(H)C₁₋₄alkylOC₁₋₄alkyl; and more preferably substituted with 1, 2, or 3 groups independently selected from the group consisting of —C₁₋₄alkyl (for example methyl), wherein each —C₁₋₄alkyl is optionally substituted by up to 3 halogen, hydroxyl or —OC₁₋₄alkyl groups; —C(O)N(H)C₁₋₄alkyl (for example —C(O)N(H)CH₃) or —C(O)N(R⁹)₂ wherein the two R⁹ and the N they are bonded to form a morpholine or pyrrolidine ring (preferably a morpholine ring), —C(O)N(H)C₁₋₄alkylOC₁₋₄alkyl and CH₂N(R¹³)S(O)₂C₁₋₄alkyl. Preferably A is selected from the group consisting optionally substituted pyrazolyl and thiazolyl.

In one preferred embodiment, A is optionally substituted with 1, 2, or 3 groups independently selected from the group consisting of —C₁₋₄alkyl (for example methyl), wherein each —C₁₋₄alkyl is optionally substituted by up to 3 halogen, hydroxyl or —OC₁₋₄alkyl groups preferably one —OC₁₋₄alkyl group; more preferably one —OCH₃ group); C(O)N(R⁹)₂ (for example —C(O)N(H)C₁₋₄alkyl or —C(O)N(R⁹)₂ wherein the two R⁹ groups and the N they are bonded to form a 4 to 7 membered non-aromatic heterocycle, the heterocycle optionally comprising 1 or 2 further heteroatoms selected from N, O and S (for example wherein the two R⁹ and the N they are bonded to form a morpholine or pyrrolidine ring)), —CH₂C(O)N(R⁹)₂ (for example —CH—₂C(O)N(H)C₁₋₄alkyl), —CH₂C(O)N(R⁹)₂ (for example —CH₂C(O)N(H)C₁₋₄alkyl), —C(O)N(R¹³)C₁₋₄alkylOC₁₋₄alkyl (for example —C(O)N(H)C₁₋₄alkylOC₁₋₄alkyl), —N(R⁹)C(O)C₁₋₄alkyl, CH₂N(R⁹)C(O)C₁₋₄alkyl and CH₂N(R¹³)S(O)₂C₁₋₄alkyl. Preferably A is selected from the group consisting optionally substituted pyrazolyl and thiazolyl.

In one preferred embodiment, A is substituted with 1, 2, or 3 groups, and at least one of the substituents is —C₁₋₄alkyl (for example methyl), wherein each —C₁₋₄alkyl is optionally substituted by up to 3 halogen, hydroxyl or —OC₁₋₄alkyl groups (preferably one —OC₁₋₄alkyl group; more preferably one —OCH₃ group); C(O)N(R⁹)₂ (for example —C(O)N(H)C₁₋₄alkyl or —C(O)N(R⁹)₂ wherein the two R⁹ groups and the N they are bonded to form a 4 to 7 membered non-aromatic heterocycle, the heterocycle optionally comprising 1 or 2 further heteroatoms selected from N, O and S (for example wherein the two R⁹ and the N they are bonded to form a morpholine or pyrrolidine ring)), —CH₂C(O)N(R⁹)₂ (for example —CH₂C(O)N(H)C₁₋₄alkyl), —CH—₂C(O)N(R⁹)₂ (for example —CH₂C(O)N(H)C₁₋₄alkyl), —C(O)N(R¹³)C₁₋₄alkylOC₁₋₄alkyl (for example —C(O)N(H)C₁₋₄alkylOC₁₋₄alkyl), —N(R⁹)C(O)C₁₋₄alkyl, —CH₂N(R¹³)₂, CH₂N(R⁹)C(O)C₁₋₄alkyl or CH₂N(H)S(O)₂C₁₋₄alkyl. Preferably A is selected from the group consisting optionally substituted pyrazolyl and thiazolyl.

In one preferred embodiment, A is substituted with 1, 2, or 3 groups, and at least one of the substituents is —CH₂N(R¹³)₂ or C₁₋₄alkyl (for example methyl), wherein each —C₁₋₄alkyl is optionally substituted by up to 3 halogen, hydroxyl or —OC₁₋₄alkyl groups (preferably one —OC₁₋₄alkyl group; more preferably one —OCH₃ group).

In another preferred embodiment, A is substituted with 1, 2, or 3 groups, and at least one of the substituents is C(O)N(R⁹)₂ (for example —C(O)N(H)C₁₋₄alkyl or —C(O)N(R⁹)₂ wherein the two R⁹ groups and the N they are bonded to form a 4 to 7 membered non-aromatic heterocycle, the heterocycle optionally comprising 1 or 2 further heteroatoms selected from N, O and S (for example wherein the two R⁹ and the N they are bonded to form a morpholine or pyrrolidine ring)), CH₂N(R⁹)C(O)C₁₋₄alkyl or CH₂N(R¹³)S(O)₂C₁₋₄alkyl (for example CH₂N(H)S(O)₂C₁₋₄alkyl).

It has been surprisingly found that where A is substituted with one carboxamide containing group, stability of the NMT inhibitor is improved. In one preferred embodiment A is substituted with 1, 2, or 3 groups, and at least one of the substituents is —C(O)N(R⁹)₂, —C(O)N(R¹³)C₁₋₄alkylOC₁₋₄alkyl, —C(O)N(C₁₋₄alkylOC₁₋₄alkyl)₂, —CH₂C(O)N(R⁹)₂, —CH—₂C(O)N(R¹³)C₁₋₄alkylOC₁₋₄alkyl, —CH₂C(O)N(C₁₋₄alkylOC₁₋₄alkyl)₂, —NHC(O)C₁₋₄alkyl, —NHC(O)CF₃, CH₂N(R¹³)C(O)C₁₋₄alkyl. In another preferred embodiment, A is substituted with 1, 2, or 3 groups, and at least one of the substituents is —C(O)N(R⁹)₂, —C(O)N(R¹³)C₁₋₄alkylOC₁₋₄alkyl, —C(O)N(C₁₋₄alkylOC₁₋₄alkyl)₂, —CH₂C(O)N(R⁹)₂, —CH₂C(O)N(R¹³)C₁₋₄alkylOC₁₋₄alkyl, —CH₂C(O)N(C₁₋₄alkylOC₁₋₄alkyl)₂, —NHC(O)C₁₋₄alkyl, —NHC(O)CF₃, CH₂N(R¹³)C(O)C₁₋₄alkyl, CO₂H, and CH₂N(H)S(O)₂C₁₋₄alkyl.

More preferably, at least one of the substituents is C(O)N(R⁹)₂ (for example —C(O)N(H)C₁₋₄alkyl or —C(O)N(R⁹)₂ wherein the two R⁹ groups and the N they are bonded to form a 4 to 7 membered non-aromatic heterocycle, the heterocycle optionally comprising 1 or 2 further heteroatoms selected from N, O and S (for example wherein the two R⁹ and the N they are bonded to form a morpholine or pyrrolidine ring)), —CH₂C(O)N(R⁹)₂ (for example —CH—₂C(O)N(H)C₁₋₄alkyl), —CH₂C(O)N(R⁹)₂ (for example —CH₂C(O)N(H)C₁₋₄alkyl), —C(O)N(R¹³)C₁₋₄alkylOC₁₋₄alkyl (for example —C(O)N(H)C₁₋₄alkylOC₁₋₄alkyl), —N(R⁹)C(O)C₁₋₄alkyl, CH₂N(R⁹)C(O)C₁₋₄alkyl or CH₂N(R¹³)S(O)₂C₁₋₄alkyl (for example CH₂N(H)S(O)₂C₁₋₄alkyl).

Even more preferably, at least one of the substituents is C(O)N(R⁹)₂ (for example —C(O)N(H)C₁₋₄alkyl) or —CH₂C(O)N(R⁹)₂ (for example —CH₂C(O)N(H)C₁₋₄alkyl). In such embodiments, preferably A is substituted pyrazolyl (such as a 4-pyrazolyl) or thiazolyl (such as a 5-thiazolyl).

In one preferred embodiment, X is O. In another preferred embodiment X is absent.

In one preferred embodiment, L is —(CH₂)ₘ— or —(CH₂)ₘO—; more preferably L is —(CH₂)ₘ—. In one preferred embodiment m is 1 or 2; preferably 2. In one preferred embodiment X is —O—; L is —(CH₂)ₘ and m is 1 or 2.

In one preferred embodiment where, for example, the NMT inhibitor is used as a diagnostic agent for the diagnosis of a disease or disorder in which inhibition of NMT provides a therapeutic or prophylactic effect, or as reference compound in a method of discovering other inhibitors of NMT, L is —(CHR¹²)ₘ— or —(CHR¹²)ₘO—; and one R¹² is a terminal C₁₋₆alkynyl optionally substituted by up to 3 —F, —Cl, —Br, I, —OH, —OCH₃, —OCF₃ or —CN groups, and more preferably one R¹² is a terminal unsubstituted C₁₋₆alkynyl. Preferably, when present, all other R¹² groups are hydrogen.

In one preferred embodiment R⁷ is hydrogen or methyl, and/or R⁸ is hydrogen or methyl.

For the avoidance of doubt, when X is absent and L is present, R¹ is a group of formula -L-A, in which group L is directly bonded to group A and to the phenyl ring shown in formula (I). When X is present and L is absent, R¹ is a group of formula —X-A, in which group X is directly bonded to group A and to the phenyl ring shown in formula (I). When X and L are both absent, R¹ is a group of formula -A, in which group A is directly bonded to the phenyl ring shown in formula (I).

In one preferred embodiment, X is O, L is —(CH₂)ₘ—, m is 1 or 2, and A is an aromatic carbocycle or heterocycle selected from the group consisting of phenyl, pyridinyl, quinolinyl, imidazolyl, benzimidazolyl, pyrazolyl, thiazolyl, 1,2,3-triazolyl and 1,2,4-triazolyl, said aromatic carbocycle or heterocycle being optionally substituted with 1, 2, or 3 groups independently selected from the group consisting of —C₁₋₄alkyl (for example methyl), wherein each —C₁₋₄alkyl is optionally substituted by up to 3 halogen, hydroxyl or —OC₁₋₄alkyl groups; —C(O)N(R⁹)₂ (for example —C(O)N(H)C₁₋₄alkyl); and —CH₂C(O)N(R⁹)₂ (for example —CH₂C(O)N(H)C₁₋₄alkyl). More preferably A is selected from the group consisting optionally substituted pyrazolyl and thiazolyl.

In one preferred embodiment, X is absent, L is —(CH₂)ₘ—, m is 3, and A is an aromatic carbocycle or heterocycle selected from the group consisting of phenyl, pyridinyl, quinolinyl, imidazolyl, benzimidazolyl, pyrazolyl, thiazolyl, 1,2,3-triazolyl and 1,2,4-triazolyl, said aromatic carbocycle or heterocycle being optionally substituted with 1, 2, or 3 groups independently selected from the group consisting of —C₁₋₄alkyl (for example methyl), wherein each —C₁₋₄alkyl is optionally substituted by up to 3 halogen, hydroxyl or —OC₁₋₄alkyl groups; —C(O)N(R⁹)₂ (for example —C(O)N(H)C₁₋₄alkyl); and —CH₂C(O)N(R⁹)₂ (for example —CH₂C(O)N(H)C₁₋₄alkyl). Preferably A is selected from the group consisting optionally substituted pyrazolyl and thiazolyl.

In one preferred embodiment, R³ and R⁴ are both hydrogen. In another preferred embodiment, R³ is hydrogen and R⁴ is methyl.

In one preferred embodiment, R⁵ and R⁶ are both methyl. In another preferred embodiment, R⁵ and R⁶ are both hydrogen. In another preferred embodiment, R⁵ is hydrogen and R⁶ is methyl.

In another embodiment, q is 0 or 1 and the R³ group and the R⁵ group and the intervening atoms form a 3 to 7 membered non-aromatic heterocycle composed of the intervening atoms and bond, or the intervening atoms and —(CHR^{a})ᵣ—. Preferably a 4 to 6 membered non-aromatic heterocycle is formed; and more preferably a 4 membered non-aromatic heterocycle. In embodiments where a 4 membered non-aromatic heterocycle is formed, preferably q is 1.

In another embodiment, q is 1 and the R¹⁰ group and the R⁵ group and the intervening atoms form a 3 to 7 membered non-aromatic heterocycle composed of the intervening atoms and —(CHR^{a})ᵣ—. Preferably a 4 to 6 membered non-aromatic heterocycle is formed.

It has been surprisingly found that stability, and in particular t_{1/2}, of the NMT inhibitors according to the invention can be improved when q is 1, and when q is 1 and the R³ group and the R⁵ group and the intervening atoms form a non-aromatic heterocycle composed of the intervening atoms and —(CHR^{a})ᵣ, for example when a 4 membered ring is formed wherein r is 1. Therefore, in one preferred embodiment q is 1 and the R³ group and the R⁵ group and the intervening atoms form a 4 membered non-aromatic heterocycle composed of the intervening atoms and —(CHR^{a})ᵣ, wherein r is 1.

It has also been surprisingly found that rapid metabolism can be achieved when q is 0. Compounds having a short t_{1/2} can be advantageous to reduce side effects and/or for administration methods in which rapid metabolism is advantageous, for example delivery by inhalation. Therefore, in another preferred embodiment q is 0.

In one preferred embodiment, A is phenyl, X is —O—; L is —(CH₂)ₘ—; m is 1 or 2; s is 0; E, J and G are each C(R⁷); K is carbon; Q is N(R⁸); M is nitrogen; R³ and R⁴ are each hydrogen; R⁵ and R⁶ are each methyl; and R⁸ is hydrogen.

In one preferred embodiment, A is 3-pyridinyl.

In one preferred embodiment, the NMT inhibitor is a compound of Formula (IA′) or (IB′) shown below:

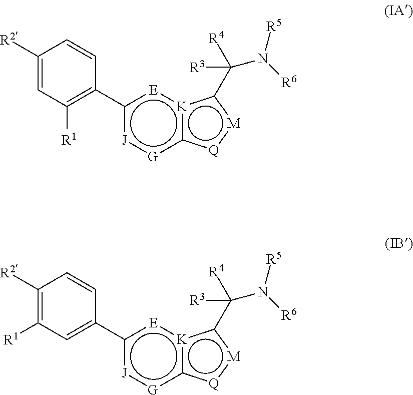

wherein R¹ is a group of formula —X-L-A; A is 3-pyridinyl; X is —O—; L is —(CH₂)ₘ—; m is 1 or 2; R^{2′} is hydrogen or fluorine; Q is N(R⁸); M is nitrogen; E, J and G are each C(R⁷); K is carbon; R³ and R⁴ are each hydrogen; R⁵ and R⁶ are each methyl; and R⁸ is hydrogen.

In one preferred embodiment, A is 4-quinolinyl. In one preferred embodiment, A is 4-quinolinyl; X is —O—; L is —(CH₂)ₘ—; m is 1 or 2; s is 0; E, J and G are each C(R⁷); K is carbon; Q is N(R⁸); M is nitrogen; R³ and R⁴ are each hydrogen; R⁵ and R⁶ are each methyl; and R⁸ is hydrogen.

In one preferred embodiment, A is 1-imidazolyl, said imidazolyl being optionally substituted by 1 or 2 methyl groups.

In one preferred embodiment, the NMT inhibitor is a compound of Formula (IA′) or (IB′) shown below:

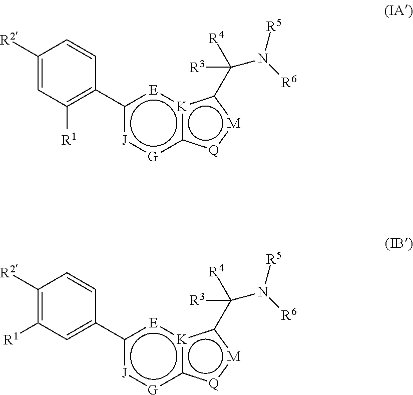

wherein R¹ is a group of formula —X-L-A; A is 1-imidazolyl, said imidazolyl being optionally substituted by 1 or 2 methyl groups; X is —O—; L is —(CH₂)ₘ—; m is 1 or 2; R^{2′} is hydrogen or fluorine; E, J and G are each C(R⁷); K is carbon; Q is N(R⁸); M is nitrogen; R³ and R⁴ are each hydrogen; R⁵ and R⁶ are each methyl; and R⁸ is hydrogen or methyl.

In one preferred embodiment, A is 1-benzimidazolyl.

In one preferred embodiment, the NMT inhibitor is a compound of Formula (IA′) shown below:

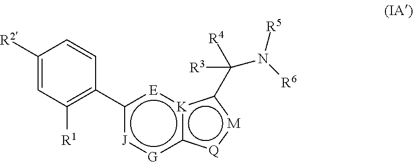

wherein R¹ is a group of formula —X-L-A; A is 1-benzimidazolyl; X is —O—; L is —(CH₂)ₘ—; m is 1 or 2; R^{2′} is fluorine or hydrogen; E, J and G are each C(R⁷); K is carbon; Q is N(R⁸); M is nitrogen; R³ and R⁴ are each hydrogen; R⁵ and R⁶ are each methyl; and R⁸ is hydrogen.

In one preferred embodiment, A is an optionally substituted 4-pyrazolyl, such as a 4-pyrazolyl optionally substituted by up to 3 substituents independently selected from the group consisting of —C₁₋₄alkyl, wherein each —C₁₋₄alkyl is optionally substituted by up to 3 halogen, hydroxyl or —OC₁₋₄alkyl groups; —C(O)N(R⁹)₂ (for example —C(O)N(H)C₁₋₄alkyl), and —CH—₂C(O)N(R⁹)₂ (for example —CH₂C(O)N(H)C₁₋₄alkyl); and R⁹ where present is each selected from the group consisting of hydrogen and —C₁₋₄alkyl, or two R⁹ groups and the N they are bonded to from a 4 to 7 membered non-aromatic heterocycle, the heterocycle optionally comprising 1 or 2 further heteroatoms selected from N, O and S. Preferably A is 4-pyrazolyl optionally substituted by up to 3-C₁₋₄alkyl; —CH₂OC₁₋₄alkyl, CF₂H, CF₃, C(O)N(Me)₂, —C(O)-1-pyrazole; or —C(O)-4-morpholine groups. More preferably A is 4-pyrazolyl substituted with one or two methyl groups and one —C₁₋₄alkyl; —CH₂OC₁₋₄alkyl, CF₂H, CF₃, C(O)N(Me)₂, —C(O)-1-pyrazole; or —C(O)-4-morpholine group.

In one preferred embodiment, the NMT inhibitor is a compound of Formula (IA″), such as, for example Formula (Iα″), shown below:

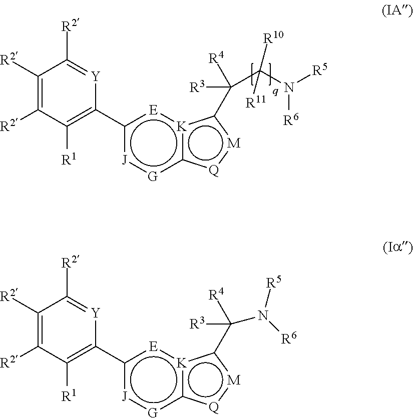

wherein R¹ is a group of formula —X-L-A; A is 4-pyrazolyl, said pyrazolyl being optionally substituted with up to 3 methyl groups; X is —O— or absent (preferably —O—); L is —(CH₂)ₘ— or —(CH₂)ₘ—O— (preferably —(CH₂)ₘ—); m is 1, 2 or 3 (preferably 1 or 2); each R^{2′} is independently selected from the group consisting of hydrogen, fluorine, chlorine, —CN and methyl; R³ and R⁴ are each hydrogen; R³ is hydrogen or methyl; R⁴ is hydrogen or methyl; R⁵ is hydrogen or methyl; R⁶ is hydrogen or methyl; when present R¹⁰ is hydrogen or methyl; when present R¹¹ is hydrogen or methyl;
or the R³ group and the R⁵ group and the intervening atoms form a 3 to 7 membered non-aromatic heterocycle composed of the intervening atoms and bond, or the intervening atoms and —(CHR^{a})ᵣ—; or the R¹⁰ group and the R⁵ group and the intervening atoms form a 3 to 7 membered non-aromatic heterocycle composed of the intervening atoms and —(CHR^{a})ᵣ—; (more preferably at least one of R⁵ and R⁶ is methyl, most preferably R⁵ and R⁶ are both methyl);
r is 1, 2, 3, 4 or 5; R^{a} is hydrogen or methyl;
R⁷ where present is hydrogen or methyl; R⁸ where present is hydrogen or methyl; and E, J, G, K, Q and M are as defined in formula (I).

Within the above embodiment, preferably:
i) E, J and G are each C(R⁷), K is carbon, Q is N(R⁸), M is nitrogen; and R⁸ is hydrogen or methyl;
ii) E, J and G are each C(R⁷), and K, Q and M are each nitrogen;
iii) E and G are each C(R⁷), and J, K, Q and M are each nitrogen;
iv) J and G are each C(R⁷), and E, K, Q and M are each nitrogen; or
v) E, J, G and M are each C(R⁷), and K and Q are each nitrogen; and more preferably E, J and G are each C(R⁷), and K, Q and M are each nitrogen; E, J and G are each C(R⁷), K is carbon, Q is N(R⁸), M is nitrogen, and R⁸ is hydrogen or methyl; or E, J, G and M are each C(R⁷), and K and Q are each nitrogen.

Also within that embodiment, preferably, Y is —CH— or —C(R^{2′})—.

Also within that embodiment, the NMT inhibitor may:

1) have formula (IA\hat{ }) shown below:

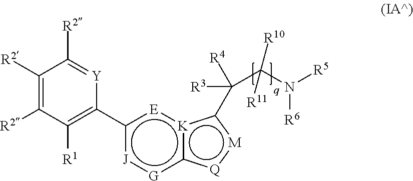

and wherein R^{2′} is selected from the group consisting of fluorine, chlorine, —CN and methyl (preferably fluorine); and R^{2″} is selected from the group consisting of hydrogen, fluorine, chlorine, —CN and methyl; or

2) have formula (IA\hat{ }\hat{ }) shown below:

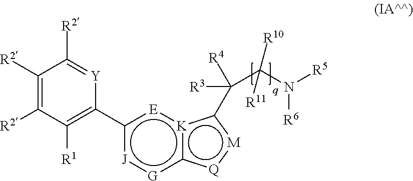

wherein R^{2′} is selected from the group consisting of fluorine, chlorine, —CN and methyl (preferably fluorine); and R^{2″} is selected from the group consisting of hydrogen, fluorine, chlorine, —CN and methyl; or

3) have the formula (IA\hat{ }\hat{ }\hat{ }) shown below:

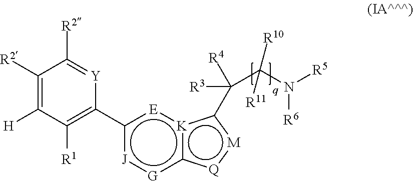

wherein R^{2′} is selected from the group consisting of fluorine, chlorine, —CN and methyl (preferably fluorine); and R^{2″} is selected from the group consisting of hydrogen, fluorine, chlorine, —CN and methyl.

In certain embodiments within the above-mentioned embodiment, R¹ is a group of formula —X-L-A; A is 4-pyrazolyl, said pyrazolyl being optionally substituted with up to 3 methyl groups; X is —O— or absent; L is —(CH₂)ₘ— or —(CH₂)ₘ—O—; m is 2; R^{2′} is selected from the group consisting of fluorine, chlorine —CN and methyl (preferably fluorine); q is 0; R³ is hydrogen or methyl; R⁴ is hydrogen or methyl; R⁵ is hydrogen or methyl; R⁶ is hydrogen or methyl; or the R³ group and the R⁵ group and the intervening atoms form a 3 to 7 membered non-aromatic heterocycle composed of the intervening atoms and bond, or the intervening atoms and —(CHR^{a})ᵣ—; (more preferably R⁵ and R⁶ are both methyl); R⁷ where present is hydrogen or methyl; R⁸ where present is hydrogen or methyl; and E, J, G, K, Q and M are as defined in formula (I); and, preferably
i) E, J and G are each C(R⁷), K is carbon, Q is N(R⁸), M is nitrogen; and R⁸ is hydrogen or methyl;
ii) E, J and G are each C(R⁷), and K, Q and M are each nitrogen;
iii) E and G are each C(R⁷), and J, K, Q and M are each nitrogen;
iv) J and G are each C(R⁷), and E, K, Q and M are each nitrogen; or
v) E, J, G and M are each C(R⁷), and K and Q are each nitrogen.

In certain embodiments of the above-mentioned embodiment, R¹ is a group of formula —X-L-A; A is 4-pyrazolyl, said pyrazolyl being optionally substituted with up to 3 methyl groups; X is —O— or absent; L is —(CH₂)ₘ— or —(CH₂)ₘ—O—; m is 2; R^{2′} is fluorine; R³ and R⁴ are each hydrogen; q is 0, R⁵ and R⁶ are each independently hydrogen or methyl (more preferably R⁵ and R⁶ are both methyl); or the R³ group and the R⁵ group and the intervening atoms form a 3 to 7 membered non-aromatic heterocycle composed of the intervening atoms and bond, or the intervening atoms and —(CHR^{a})ᵣ—; R⁷ where present is hydrogen or methyl; R⁸ is methyl; K is carbon; Q is N(R⁸); M is nitrogen; and E, J and G are as defined in formula (I); or

R¹ is a group of formula —X-L-A; A is 4-pyrazolyl, said pyrazolyl being optionally substituted with up to 3 methyl groups; X is —O— or absent; L is —(CH₂)ₘ— or —(CH₂)ₘ—O—; m is 2; R^{2′} is fluorine; R³ and R⁴ are each hydrogen; R⁵ and R⁶ are each independently hydrogen or methyl (more preferably R⁵ and R⁶ are both methyl); R⁷ where present is hydrogen or methyl; Q, M and K are each nitrogen; and E, J and G are each C(R⁷).

In one preferred embodiment, the NMT inhibitor is a compound of Formula (IA′) shown below:

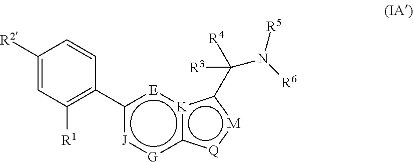

wherein R¹ is a group of formula —X-L-A; A is 4-pyrazolyl, said pyrazolyl being optionally substituted with up to 3 methyl groups; X is —O— or absent; L is —(CH₂)ₘ— or —(CH₂)ₘ—O—; m is 2; R^{2′} is selected from the group consisting of fluorine, chlorine —CN and methyl (preferably fluorine); R³ is hydrogen or methyl; R⁴ is hydrogen or methyl; R⁵ is hydrogen or methyl; R⁶ is hydrogen or methyl; or
the R³ group and the R⁵ group and the intervening atoms form a 3 to 7 membered non-aromatic heterocycle composed of the intervening atoms and bond, or the intervening atoms and —(CHR^{a})ᵣ—; (more preferably R⁵ and R⁶ are both methyl); R⁷ where present is hydrogen or methyl; R⁸ where present is hydrogen or methyl; and E, J, G, K, Q and M are as defined in formula (I).

Within that embodiment, preferably:
i) E, J and G are each C(R⁷), K is carbon, Q is N(R⁸), M is nitrogen; and R⁸ is hydrogen or methyl;
ii) E, J and G are each C(R⁷), and K, Q and M are each nitrogen;
iii) E and G are each C(R⁷), and J, K, Q and M are each nitrogen;
iv) J and G are each C(R⁷), and E, K, Q and M are each nitrogen; or
v) E, J, G and M are each C(R⁷), and K and Q are each nitrogen.

In one preferred embodiment, the NMT inhibitor is a compound of Formula (IA′) shown below:

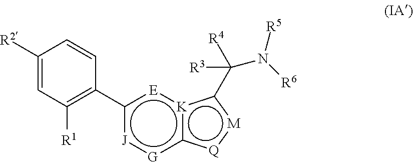

wherein R¹ is a group of formula —X-L-A; A is 4-pyrazolyl, said pyrazolyl being optionally substituted with up to 3 methyl groups; X is —O— or absent; L is —(CH₂)ₘ— or —(CH₂)ₘ—O—; m is 2; R^{2′} is fluorine; R³ and R⁴ are each hydrogen; R⁵ and R⁶ are each independently hydrogen or methyl (more preferably R⁵ and R⁶ are both methyl); R⁷ where present is hydrogen or methyl; R⁸ is methyl; K is carbon; Q is N(R⁸); M is nitrogen; and E, J and G are as defined in formula (I).

In one preferred embodiment, the NMT inhibitor is a compound of Formula (IA′) shown below:

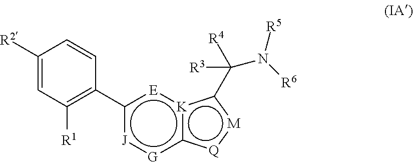

wherein R¹ is a group of formula —X-L-A; A is 4-pyrazolyl, said pyrazolyl being optionally substituted with up to 3 methyl groups; X is —O— or absent; L is —(CH₂)ₘ— or —(CH₂)ₘ—O—; m is 2; R^{2′} is fluorine; R³ and R⁴ are each hydrogen; R⁵ and R⁶ are each independently hydrogen or methyl (more preferably R⁵ and R⁶ are both methyl); R⁷ where present is hydrogen or methyl; Q, M and K are each nitrogen; and E, J and G are each C(R⁷).

In one preferred embodiment, the NMT inhibitor is a compound Formula (IA″″) shown below:

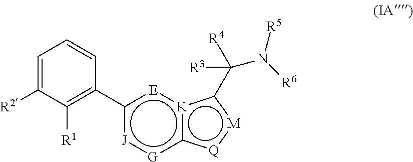

wherein R¹ is a group of formula —X-L-A; A is 4-pyrazolyl, said pyrazolyl being optionally substituted with up to 3 methyl groups; X is —O— or absent; L is —(CH₂)ₘ— or —(CH₂)ₘ—O—; m is 2; R^{2′} is selected from the group consisting of fluorine, —CN and methyl; R³ and R⁴ are each hydrogen; R⁵ and R⁶ are each independently hydrogen or methyl (more preferably R⁵ and R⁶ are both methyl); R⁷ where present is hydrogen or methyl; R⁸ where present is hydrogen or methyl; and E, J, G, K, Q and M are as defined in formula (I).

Within that embodiment, preferably:
i) E, J and G are each C(R⁷), K is carbon, Q is N(R⁸), M is nitrogen; and R⁸ is hydrogen or methyl;
ii) E, J and G are each C(R⁷), and K, Q and M are each nitrogen;
iii) E and G are each C(R⁷), and J, K, Q and M are each nitrogen;
iv) J and G are each C(R⁷), and E, K, Q and M are each nitrogen; or
v) E, J, G and M are each C(R⁷), and K and Q are each nitrogen.

In one preferred embodiment, the compound has the formula (IA″″)

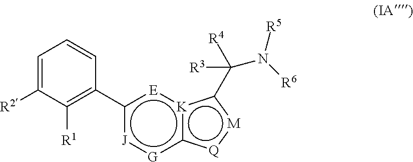

wherein R¹ is a group of formula —X-L-A; A is 4-pyrazolyl, said pyrazolyl being optionally substituted with up to 3 methyl groups; X is —O— or absent; L is —(CH₂)ₘ— or —(CH₂)ₘ—O—; m is 2; R^{2′} is fluorine; R³ and R⁴ are each hydrogen; R⁵ and R⁶ are each independently hydrogen or methyl (more preferably R⁵ and R⁶ are both methyl); R⁷ where present is hydrogen or methyl; R⁸ is methyl; K is carbon; Q is N(R⁸); M is nitrogen; and E, J and G are as defined in formula (I).

In one preferred embodiment, the NMT inhibitor is a compound of Formula (IA″″) shown below:

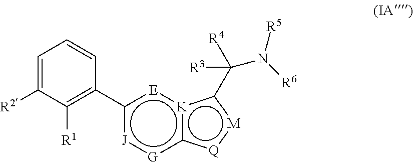

wherein R¹ is a group of formula —X-L-A; A is 4-pyrazolyl, said pyrazolyl being optionally substituted with up to 3 methyl groups; X is —O— or absent; L is —(CH₂)ₘ— or —(CH₂)ₘ—O—; m is 2; R^{2′} is fluorine; R³ and R⁴ are each hydrogen; R⁵ and R⁶ are each independently hydrogen or methyl (more preferably R⁵ and R⁶ are both methyl); R⁷ where present is hydrogen or methyl; Q, M and K are each nitrogen; and E, J and G are each C(R⁷).

In one preferred embodiment, A is an optionally substituted 5-thiazolyl, such as a 5-thiazolyl optionally substituted by 1 or 2 methyl groups.

In one preferred embodiment, the NMT inhibitor is a compound of Formula (IA″), such as, for example, a compound of Formula (Iα″), shown below:

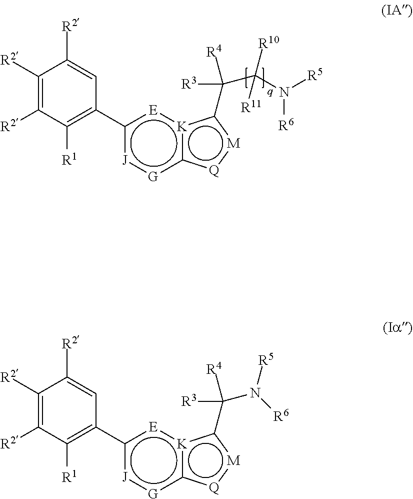

or the NMT inhibitor is a compound of Formula (IA′) shown below:

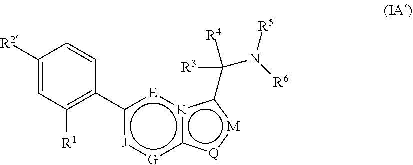

wherein R¹ is a group of formula —X-L-A; A is 5-thiazolyl optionally substituted with 1 or 2 methyl groups (more preferably A is 5-thiazolyl substituted with one methyl group at the 4-position; or substituted with two methyl groups, one at the 2-position and one at the 4-position); X is —O—; L is —(CH₂)ₘ—; m is 1, 2 or 3 (preferably 1 or 2); R^{2′} is hydrogen, chlorine or fluorine (preferably fluorine); R³ is hydrogen or methyl; R⁴ is hydrogen or methyl; R⁵ is hydrogen or methyl; R⁶ is hydrogen or methyl; when present R¹⁰ is hydrogen or methyl; when present R¹¹ is hydrogen or methyl;
or the R³ group and the R⁵ group and the intervening atoms form a 3 to 7 membered non-aromatic heterocycle composed of the intervening atoms and bond, or the intervening atoms and —(CHR^{a})ᵣ—; or the R¹⁰ group and the R⁵ group and the intervening atoms form a 3 to 7 membered non-aromatic heterocycle composed of the intervening atoms and —(CHR^{a})ᵣ—; r is 1, 2, 3, 4 or 5; R^{a} is hydrogen or methyl (preferably R³ and R⁴ are each independently hydrogen or methyl; and R⁵ and R⁶ are each independently hydrogen or methyl;) K is carbon; Q is N(R⁸); M is nitrogen; R⁸ is methyl; and E, J and G are as defined in formula (I).

Also within that embodiment, preferably, Y is —CH— or —C(R^{2′})—.

Also within that embodiment, the NMT inhibitor may:

1) have formula (IA\hat{ }) shown below:

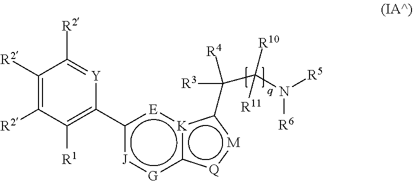

wherein R^{2′} is selected from the group consisting of fluorine and chlorine (preferably fluorine); and R^{2″} is selected from the group consisting of hydrogen, fluorine and chlorine;

2) have formula (IA\hat{ }\hat{ }) shown below:

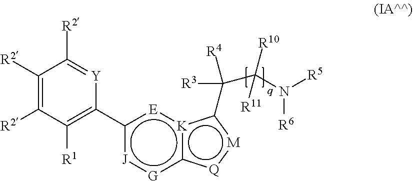

wherein R^{2′} is selected from the group consisting of fluorine and chlorine (preferably fluorine); and R^{2″} is selected from the group consisting of hydrogen, fluorine and chlorine; or

3) have formula (IA\hat{ }\hat{ }\hat{ }) shown below:

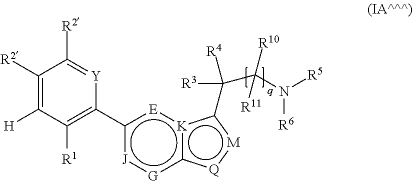

wherein R^{2′} is selected from the group consisting of fluorine and chlorine (preferably fluorine); and R^{2″} is selected from the group consisting of hydrogen, fluorine and chlorine.

In one preferred embodiment, the NMT inhibitor is a compound of Formula (IA′) shown below:

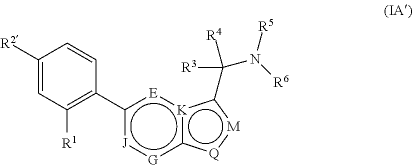

wherein R¹ is a group of formula —X-L-A; A is 5-thiazolyl optionally substituted with 1 or 2 methyl groups (more preferably A is 5-thiazolyl substituted with one methyl group at the 4-position; or substituted with two methyl groups, one at the 2-position and one at the 4-position); X is —O—; L is —(CH₂)ₘ—; m is 1, 2 or 3 (preferably 3); R^{2′} is hydrogen, chlorine or fluorine (preferably fluorine); R³ is hydrogen or methyl; R⁴ is hydrogen or methyl; R⁵ is hydrogen or methyl; R⁶ is hydrogen or methyl; when present R¹⁰ is hydrogen or methyl; when present R¹¹ is hydrogen or methyl; or the R³ group and the R⁵ group and the intervening atoms form a 3 to 7 membered non-aromatic heterocycle composed of the intervening atoms and bond, or the intervening atoms and —(CHR^{a})ᵣ—; or the R¹⁰ group and the R⁵ group and the intervening atoms form a 3 to 7 membered non-aromatic heterocycle composed of the intervening atoms and —(CHR^{a})ᵣ—; r is 1, 2, 3, 4 or 5; R^{a} is hydrogen or methyl (preferably R³ and R⁴ are each independently hydrogen or methyl; and R⁵ and R⁶ are each independently hydrogen or methyl); K, Q and M are each nitrogen; and E, J and G are as defined in formula (I).

In one preferred embodiment, A is selected from the group consisting of optionally substituted 1,2,4-triazol-1-yl, optionally substituted 1,2,4-triazol-4-yl, optionally substituted 1,2,4-triazol-3-yl and optionally substituted 1,2,3-triazol-4-yl. Within that embodiment, preferably X is absent and L is —(CH₂)₃—. A is preferably optionally substituted 1,2,4-triazol-1-yl.

In one preferred embodiment, the NMT inhibitor is a compound of Formula (IA′″) shown below:

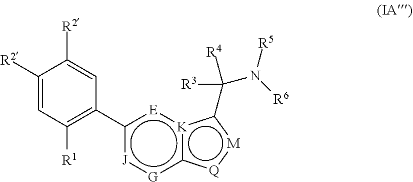

wherein R¹ is a group of formula —X-L-A; A is selected from the group consisting of optionally substituted 1,2,4-triazol-1-yl, optionally substituted 1,2,4-triazol-4-yl, optionally substituted 1,2,4-triazol-3-yl and optionally substituted 1,2,3-triazol-4-yl; X is —O— or absent; L is —(CH₂)ₘ—; m is 2 or 3; R^{2′} is hydrogen or fluorine (more preferably R^{2′} is fluorine); R^{2′} is hydrogen or —OCH₃; R³ and R⁴ are each hydrogen; R⁵ and R⁶ are each methyl; K is carbon; Q is N(R⁸); M is nitrogen; R⁸ is methyl or hydrogen; and E, J and G are as defined in formula (I). Within that embodiment, preferably X is absent and L is —(CH₂)₃—.

In one preferred embodiment, the NMT inhibitor is a compound of Formula (IA′″) shown below:

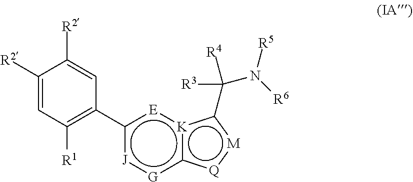

wherein R¹ is a group of formula —X-L-A; A is selected from the group consisting of optionally substituted 1,2,4-triazol-1-yl, optionally substituted 1,2,4-triazol-4-yl, optionally substituted 1,2,4-triazol-3-yl and optionally substituted 1,2,3-triazol-4-yl; X is —O— or absent; L is —(CH₂)ₘ—; m is 2 or 3; R^{2′} is hydrogen or fluorine (more preferably R^{2′} is fluorine); R^{2′} is hydrogen or —OCH₃; R³ and R⁴ are each hydrogen; R⁵ and R⁶ are each methyl; K, Q and M are each nitrogen; and E, J and G are as defined in formula (I). Within that embodiment, preferably X is absent and L is —(CH₂)₃—.

In one preferred embodiment, the NMT inhibitor is a compound of Formula (IA′″) shown below:

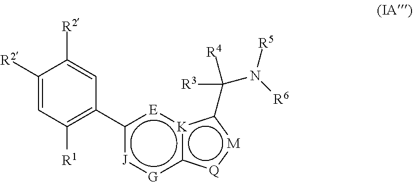

wherein R¹ is a group of formula —X-L-A; A is selected from the group consisting of optionally substituted 1,2,4-triazol-1-yl, optionally substituted 1,2,4-triazol-4-yl, optionally substituted 1,2,4-triazol-3-yl and optionally substituted 1,2,3-triazol-4-yl; X is absent; L is —(CH₂)₃—; R^{2′} is fluorine; R^{2′} is hydrogen or —OCH₃; R³ and R⁴ are each hydrogen; R⁵ and R⁶ are each methyl; K, Q and M are each nitrogen; and E, J and G are as defined in formula (I).

In one embodiment, the NMT inhibitor is a compound of Formula (IB) shown below:

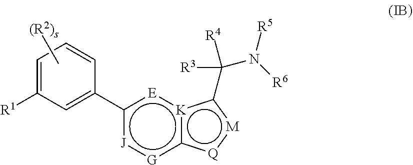

wherein R¹ is a group of formula —X-L-A; X is —O—; L is —(CH₂)ₘ—; m is 1 or 2; A is selected from the group consisting of optionally substituted 3-pyridinyl, 4-pyridinyl and 1-imidazolyl; s is 0; R³ and R⁴ are each hydrogen; R⁵ and R⁶ are each methyl; K is carbon; Q is N(R⁸); M is nitrogen; and R⁸ is hydrogen; and E, J, and G are as defined in formula (I).

In one embodiment, the NMT inhibitor is a compound of Formula (IB), such as, for example, a compound of Formula (Iβ), shown below:

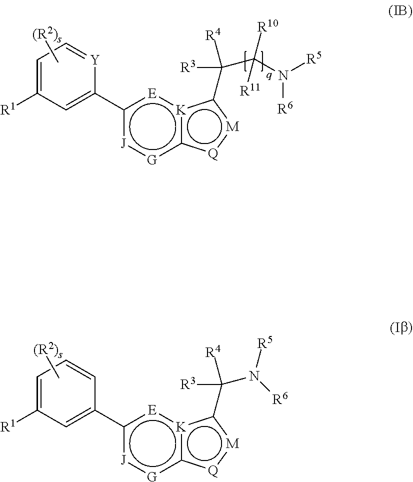

wherein R¹ is a group of formula —X-L-A; X is —O—; L is —(CH₂)ₘ—; m is 1 or 2; A is selected from the group consisting of optionally substituted 3-pyridinyl, 4-pyridinyl and 1-imidazolyl; s is 0 or 1; R^{2′} is fluorine; q is 0 or 1 (preferably q is 0); R³ and R⁴ are each hydrogen; R⁵ and R⁶ are each methyl; R¹⁰ and R¹¹ are each hydrogen or methyl; K, Q and M are each nitrogen; and E, J, and G are as defined in formula (I).

In one embodiment, the NMT inhibitor is any one of the following compounds:

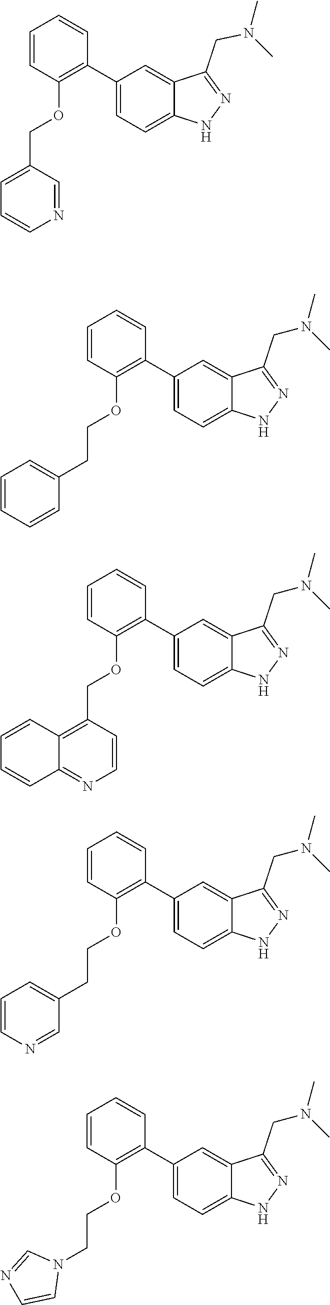 [SMILES: CN(C)Cc1n[nH]c2ccc(-c3ccccc3OCc3cccnc3)cc12 ; CN(C)Cc1n[nH]c2ccc(-c3ccccc3OCCc3ccccc3)cc12 ; CN(C)Cc1n[nH]c2ccc(-c3ccccc3OCc3ccnc4ccccc34)cc12 ; CN(C)Cc1n[nH]c2ccc(-c3ccccc3OCCc3cccnc3)cc12 ; CN(C)Cc1n[nH]c2ccc(-c3ccccc3OCCn3ccnc3)cc12]

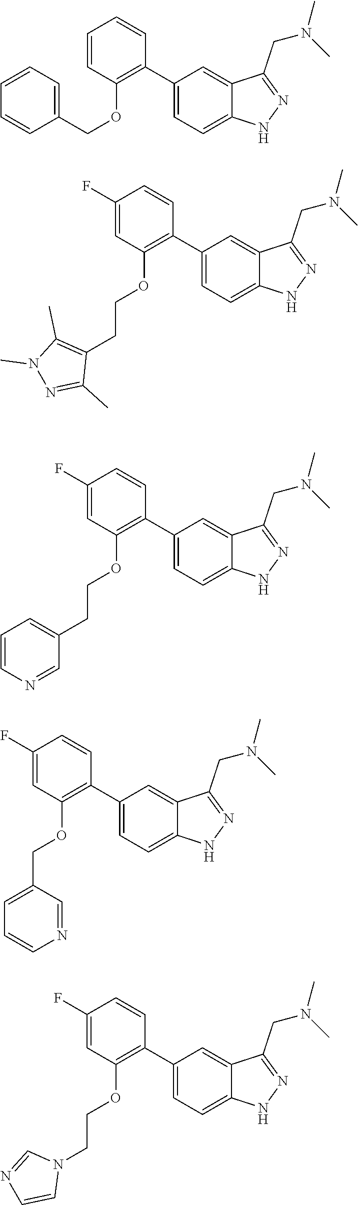

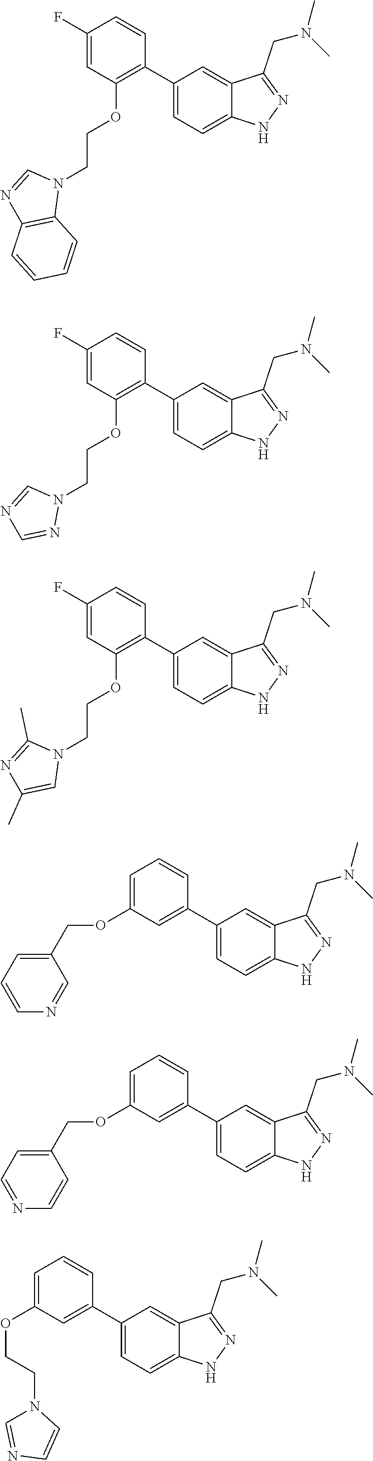 [SMILES: CN(C)Cc1n[nH]c2ccc(-c3ccc(F)cc3OCCn3cnc4ccccc43)cc12 ; CN(C)Cc1n[nH]c2ccc(-c3ccc(F)cc3OCCn3cncn3)cc12 ; Cc1cn(CCOc2cc(F)ccc2-c2ccc3[nH]nc(CN(C)C)c3c2)c(C)n1 ; CN(C)Cc1n[nH]c2ccc(-c3cccc(OCc4cccnc4)c3)cc12 ; CN(C)Cc1n[nH]c2ccc(-c3cccc(OCc4ccncc4)c3)cc12 ; CN(C)Cc1n[nH]c2ccc(-c3cccc(OCCn4ccnc4)c3)cc12]

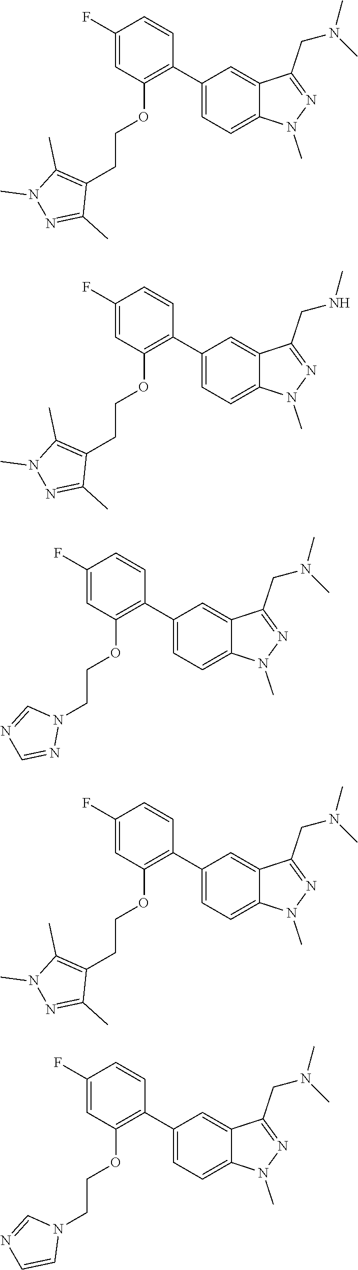

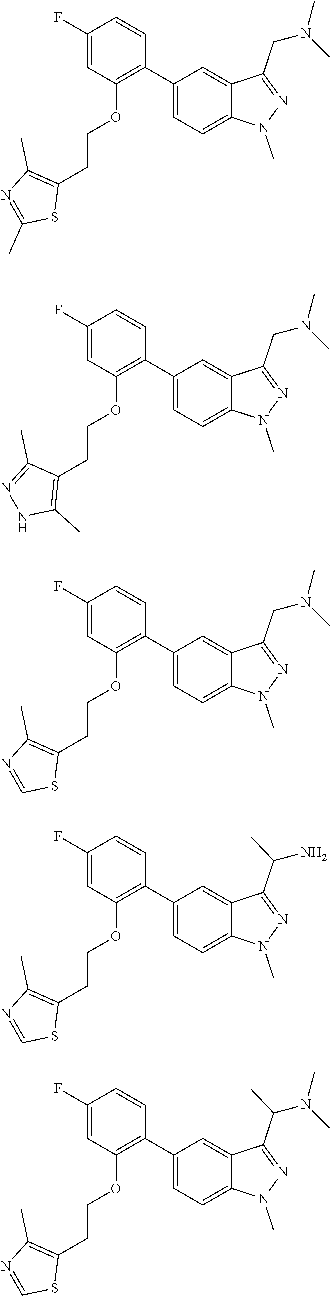 [SMILES: Cc1nc(C)c(CCOc2cc(F)ccc2-c2ccc3c(c2)c(CN(C)C)nn3C)s1 ; Cc1n[nH]c(C)c1CCOc1cc(F)ccc1-c1ccc2c(c1)c(CN(C)C)nn2C ; Cc1ncsc1CCOc1cc(F)ccc1-c1ccc2c(c1)c(CN(C)C)nn2C ; Cc1ncsc1CCOc1cc(F)ccc1-c1ccc2c(c1)c(C(C)N)nn2C ; Cc1ncsc1CCOc1cc(F)ccc1-c1ccc2c(c1)c(C(C)N(C)C)nn2C]

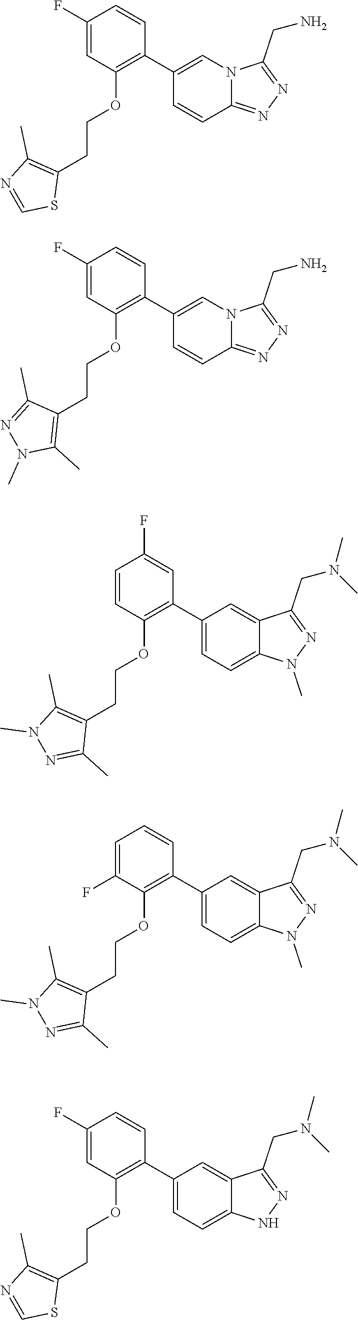

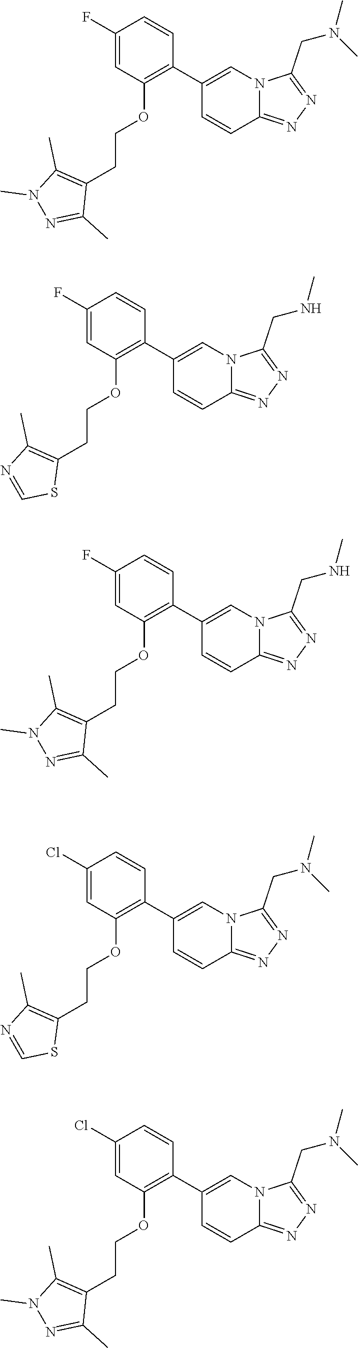

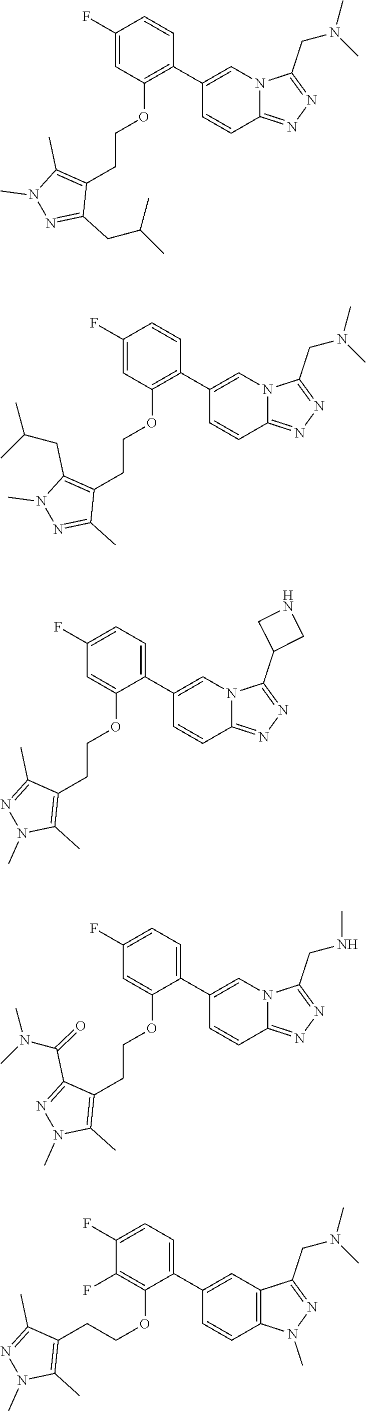

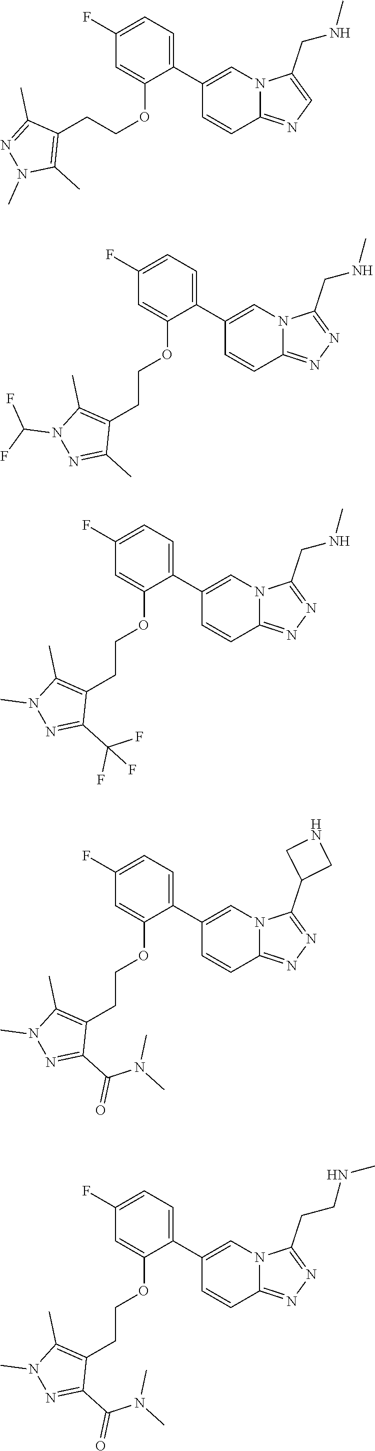

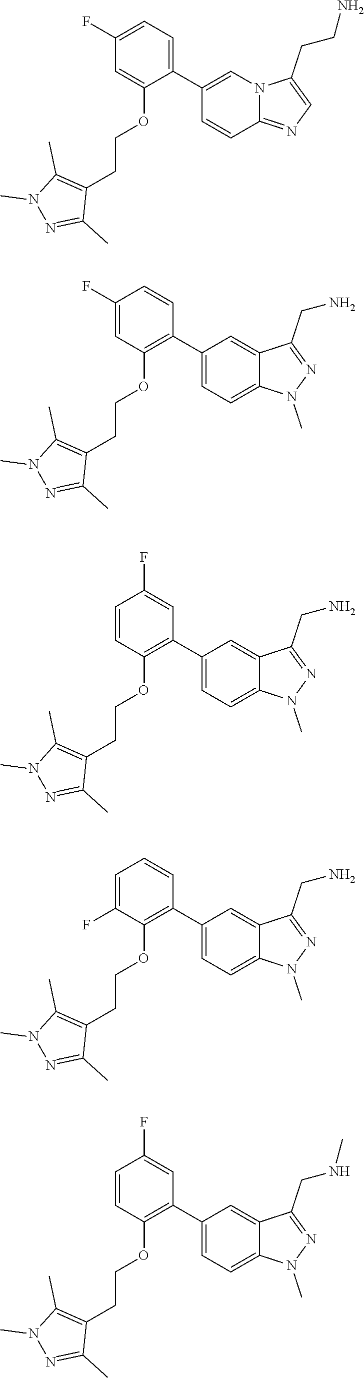

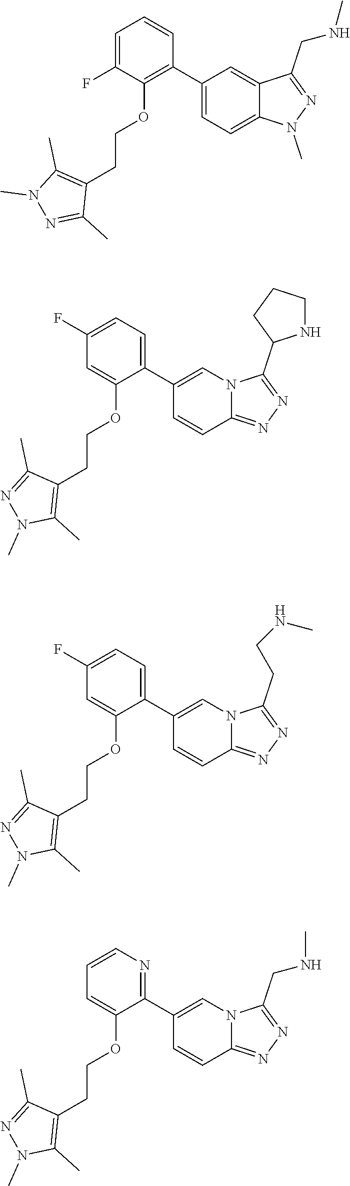

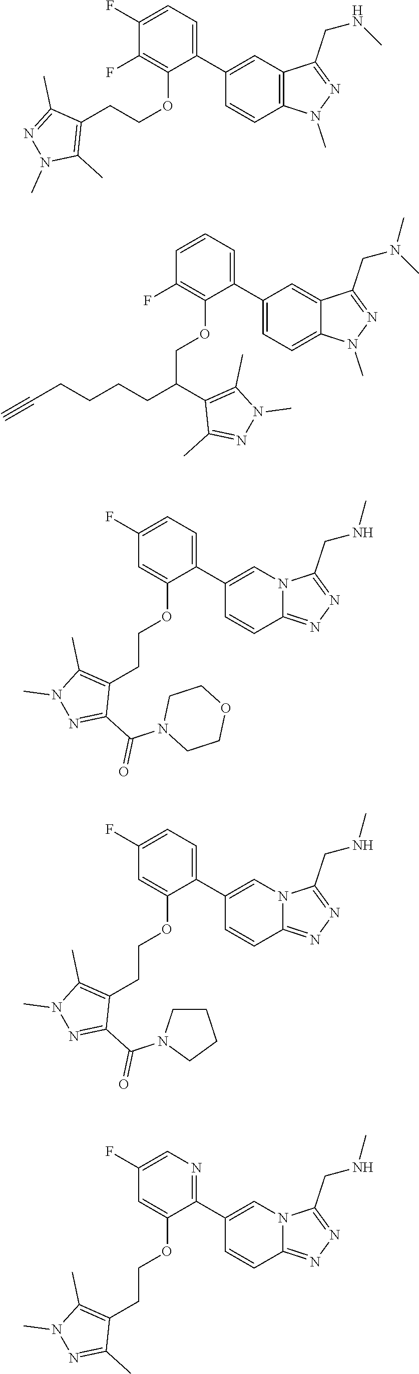

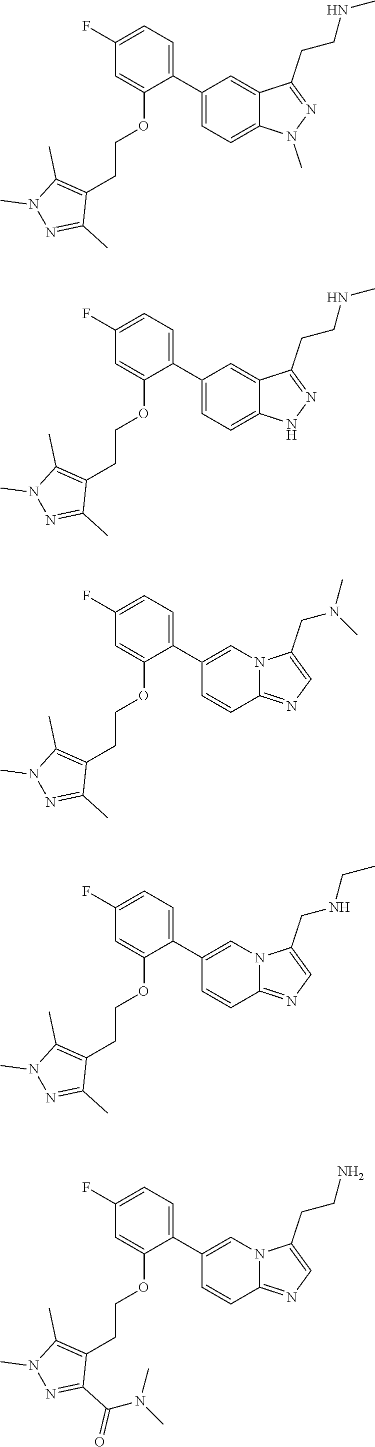

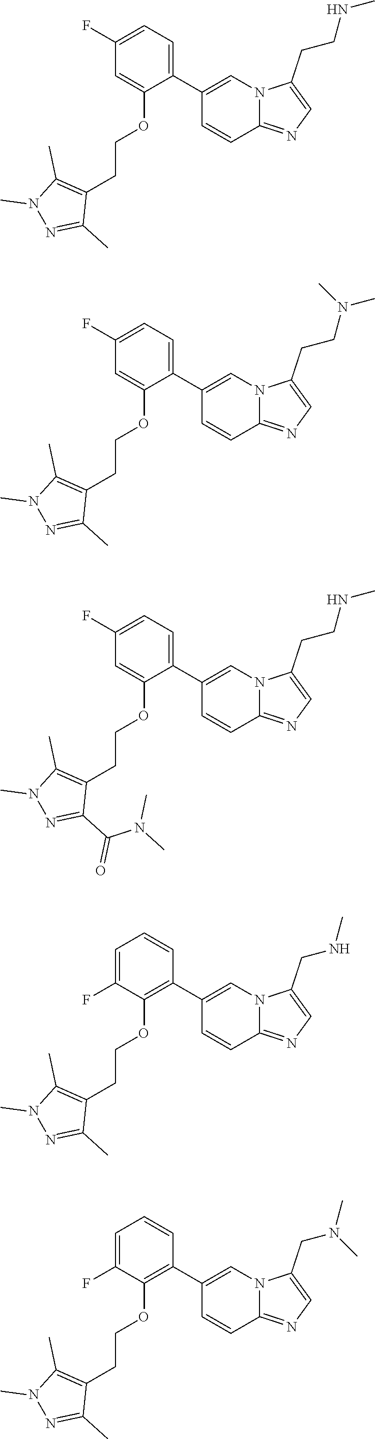

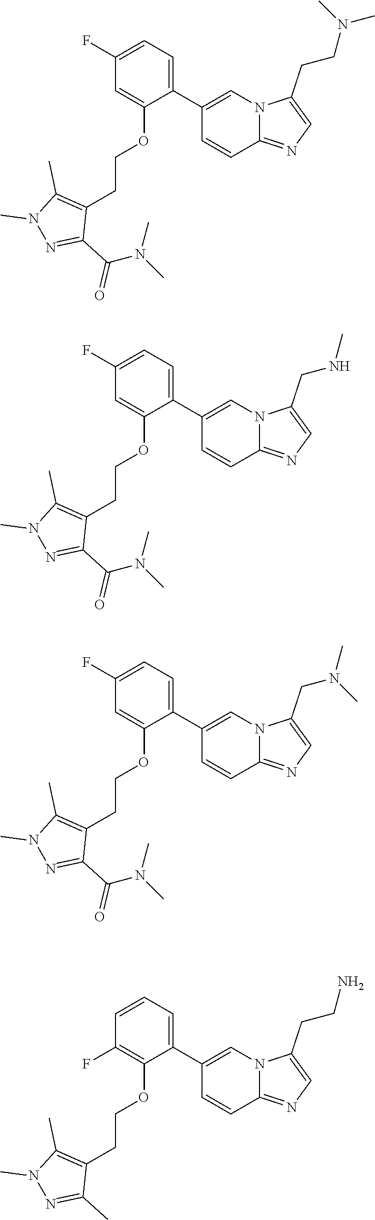

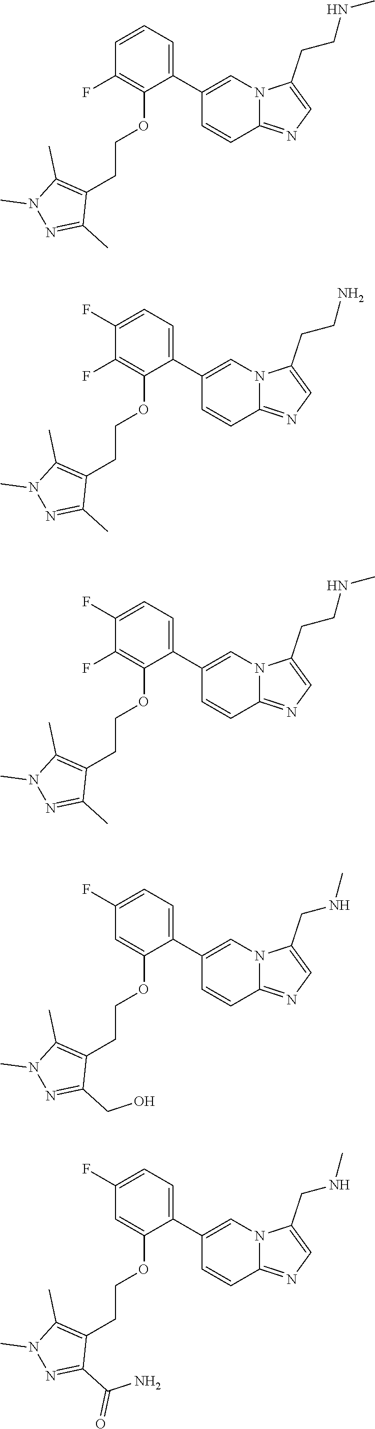

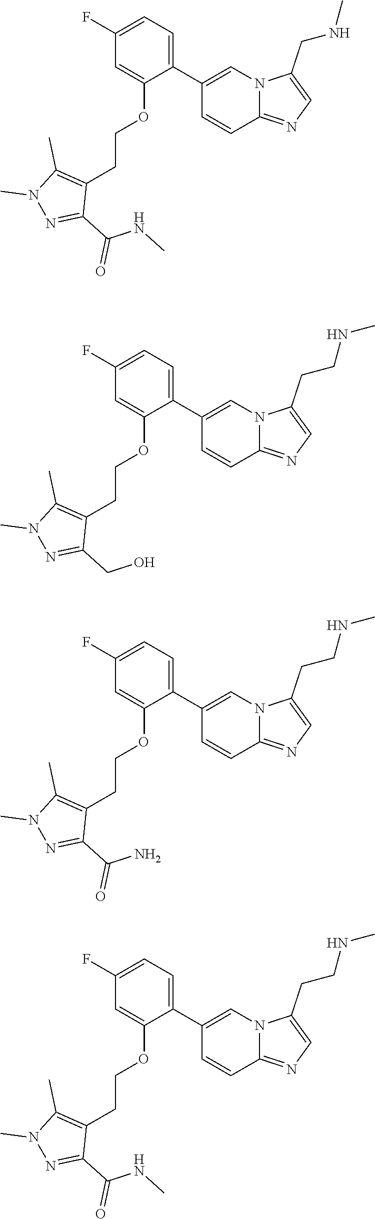

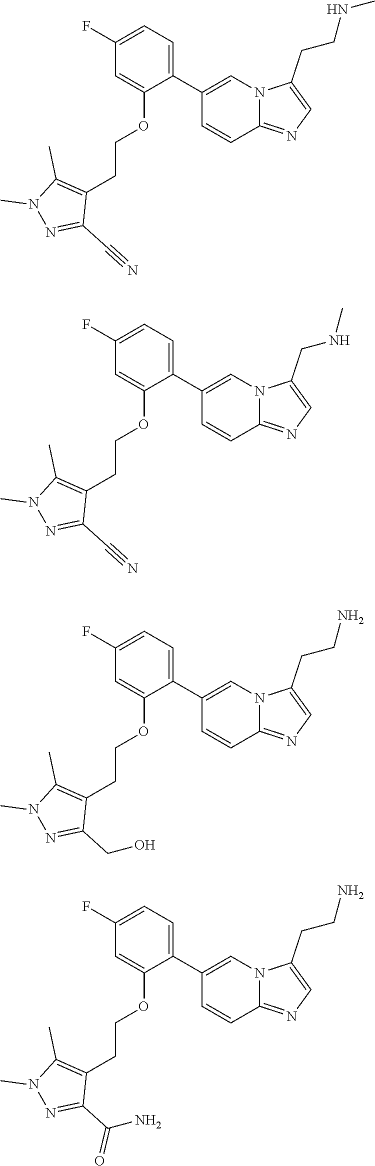

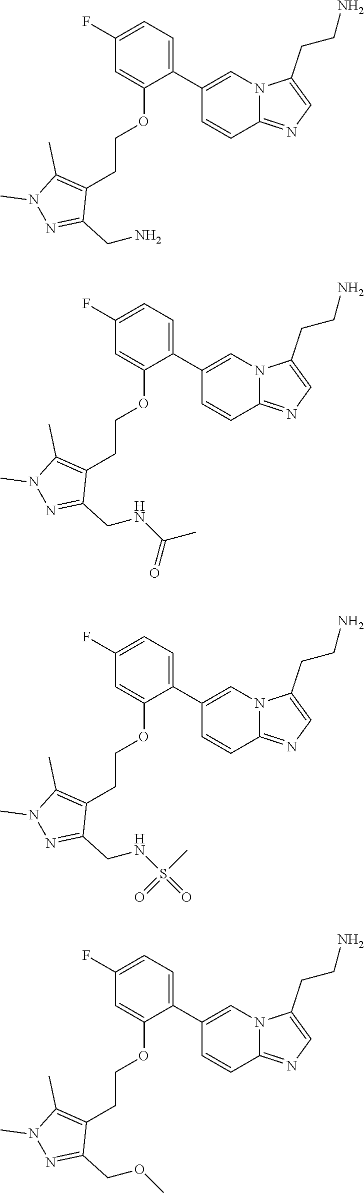 [SMILES: Cc1c(CCOc2cc(F)ccc2-c2ccc3ncc(CCN)n3c2)c(CN)nn1C ; CC(=O)NCc1nn(C)c(C)c1CCOc1cc(F)ccc1-c1ccc2ncc(CCN)n2c1 ; Cc1c(CCOc2cc(F)ccc2-c2ccc3ncc(CCN)n3c2)c(CNS(C)(=O)=O)nn1C ; COCc1nn(C)c(C)c1CCOc1cc(F)ccc1-c1ccc2ncc(CCN)n2c1]

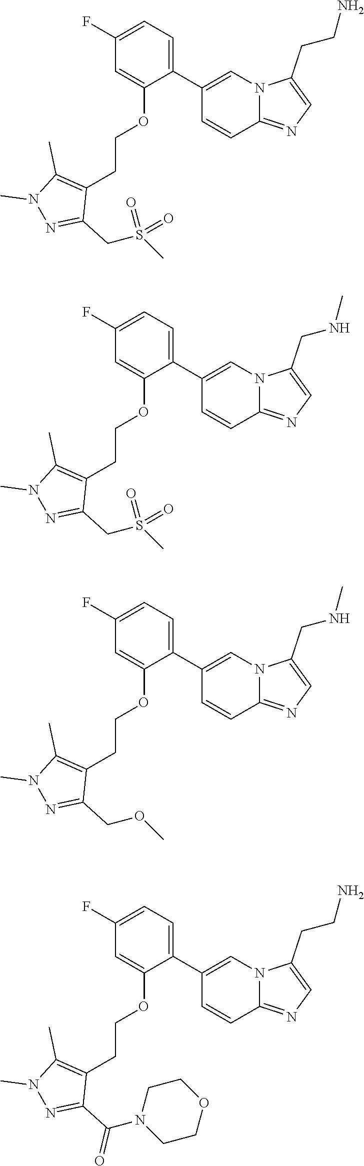

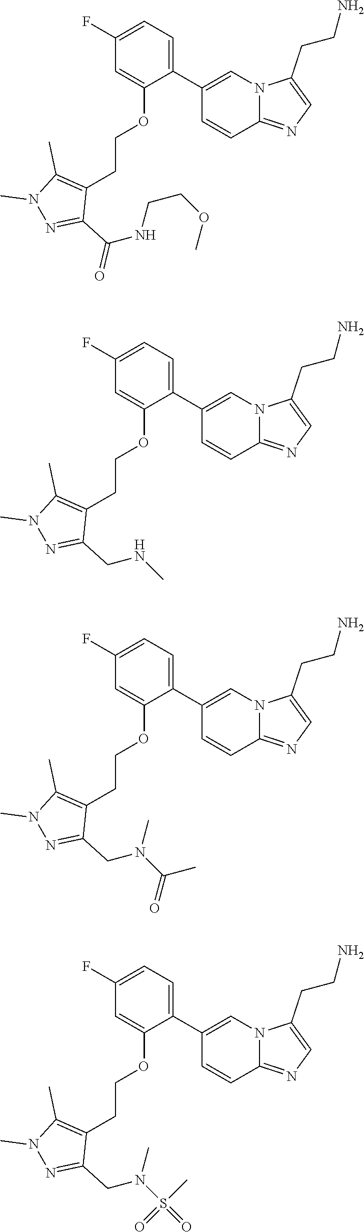 [SMILES: COCCNC(=O)c1nn(C)c(C)c1CCOc1cc(F)ccc1-c1ccc2ncc(CCN)n2c1 ; CNCc1nn(C)c(C)c1CCOc1cc(F)ccc1-c1ccc2ncc(CCN)n2c1 ; CC(=O)N(C)Cc1nn(C)c(C)c1CCOc1cc(F)ccc1-c1ccc2ncc(CCN)n2c1 ; Cc1c(CCOc2cc(F)ccc2-c2ccc3ncc(CCN)n3c2)c(CN(C)S(C)(=O)=O)nn1C]

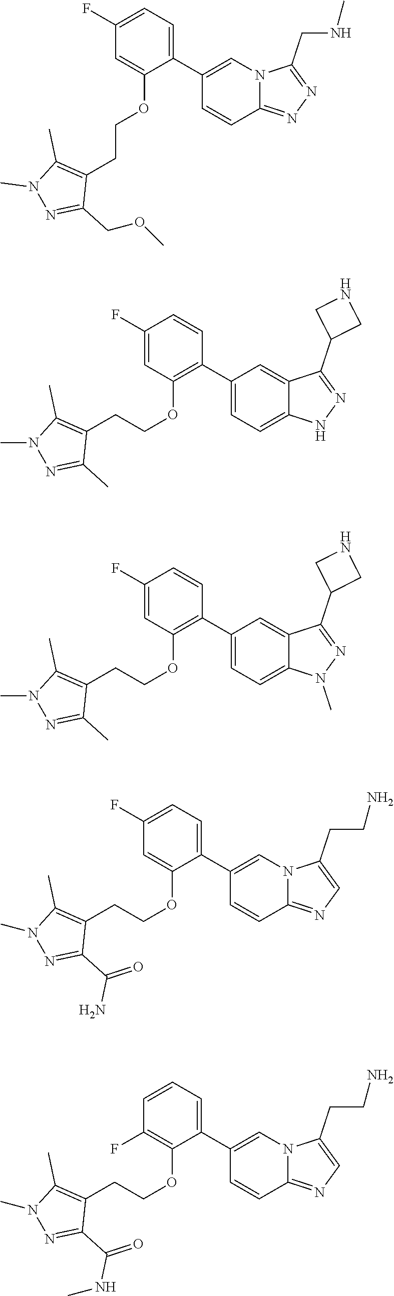

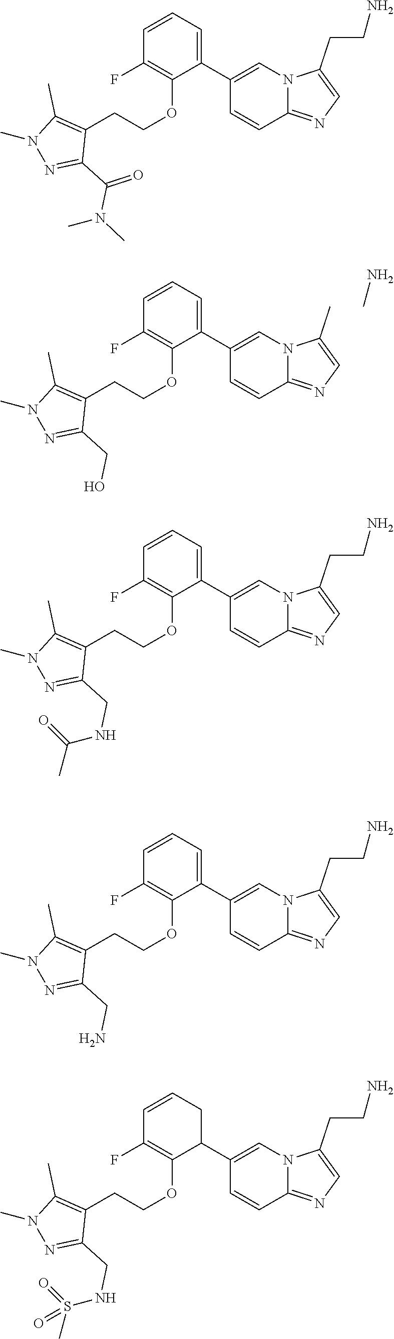

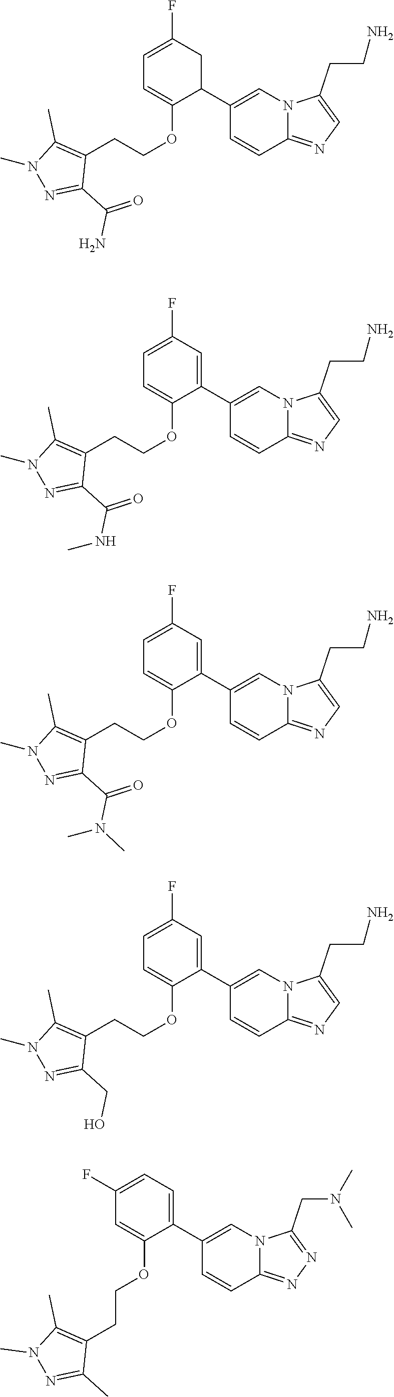

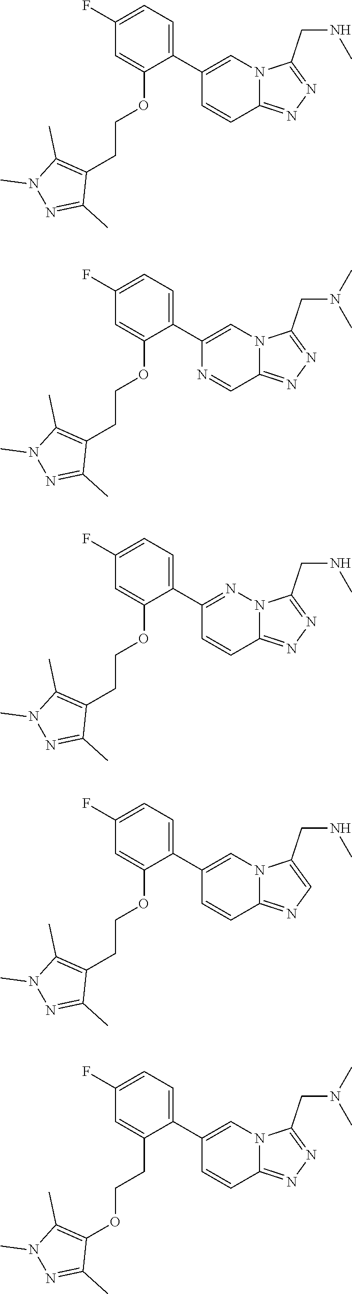

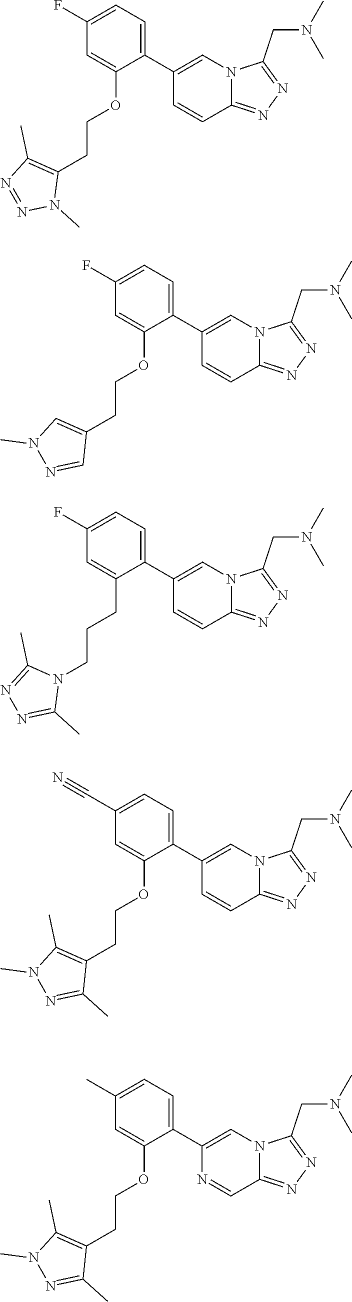

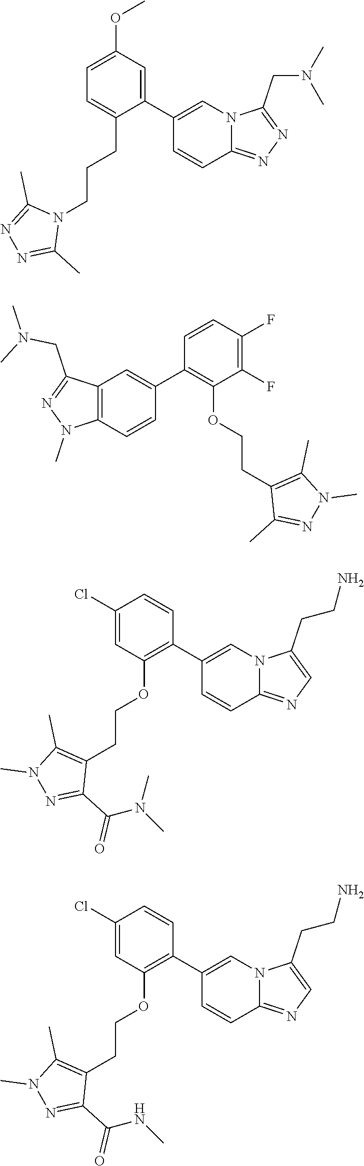

or a pharmaceutically acceptable salt, hydrate or solvate thereof.

In another embodiment, the NMT inhibitor is any one of the following compounds:

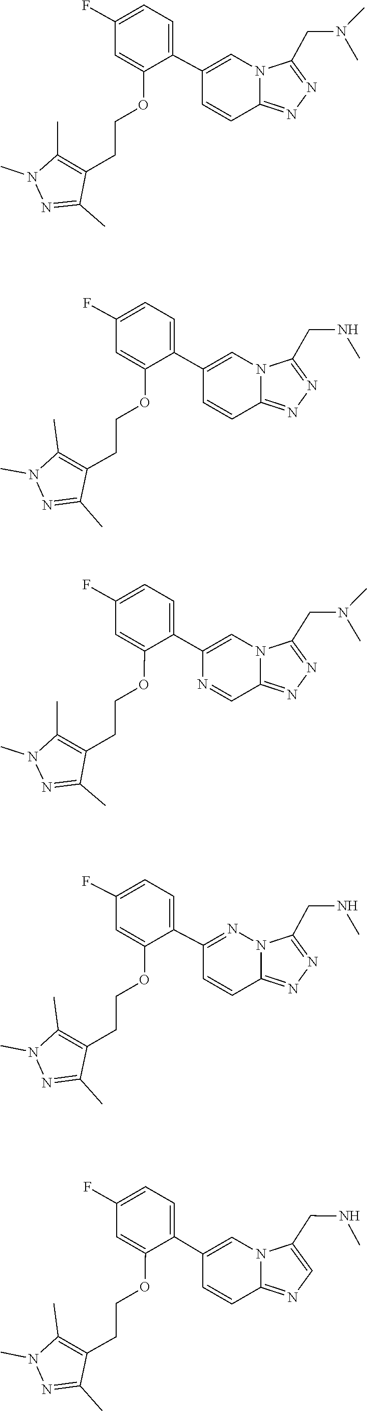

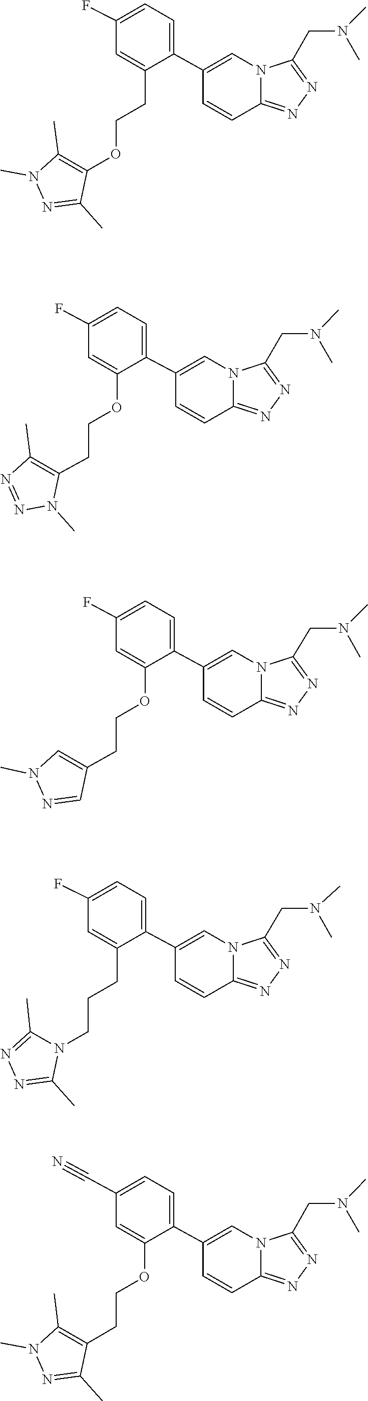

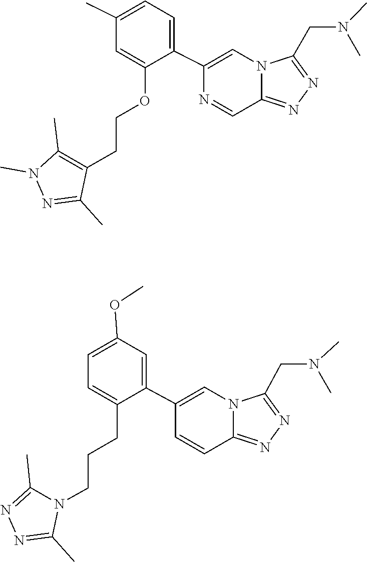

or a pharmaceutically acceptable salt, hydrate or solvate thereof.

In another embodiment, the NMT inhibitor is any one of the following compounds:

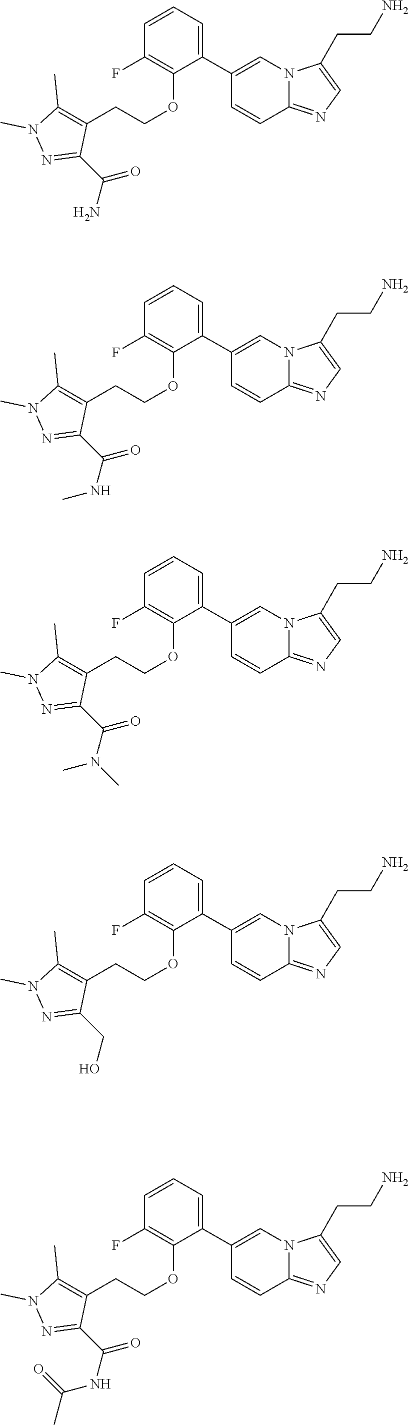 [SMILES: Cc1c(CCOc2c(F)cccc2-c2ccc3ncc(CCN)n3c2)c(C(N)=O)nn1C ; CNC(=O)c1nn(C)c(C)c1CCOc1c(F)cccc1-c1ccc2ncc(CCN)n2c1 ; Cc1c(CCOc2c(F)cccc2-c2ccc3ncc(CCN)n3c2)c(C(=O)N(C)C)nn1C ; Cc1c(CCOc2c(F)cccc2-c2ccc3ncc(CCN)n3c2)c(CO)nn1C ; CC(=O)NC(=O)c1nn(C)c(C)c1CCOc1c(F)cccc1-c1ccc2ncc(CCN)n2c1]

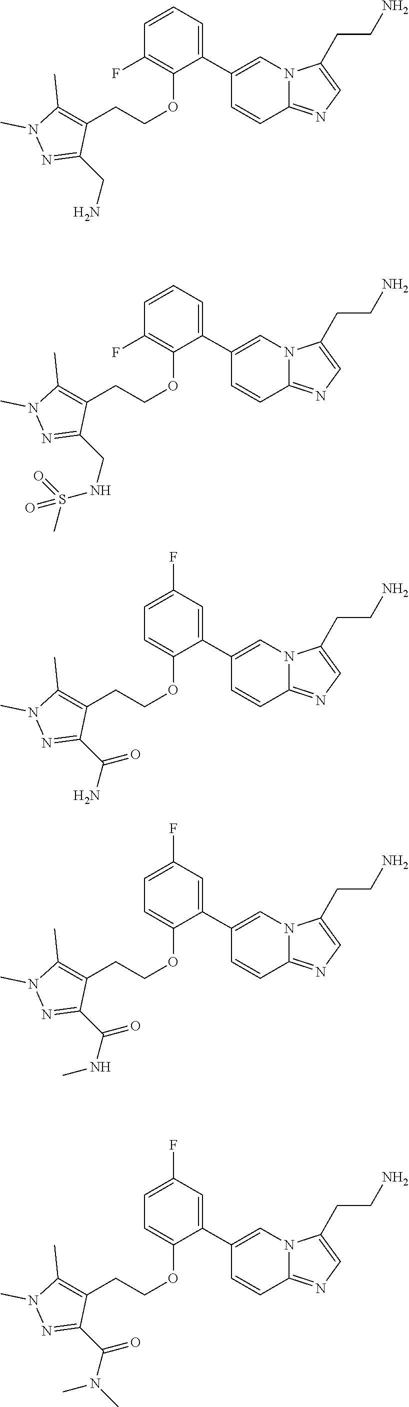 [SMILES: Cc1c(CCOc2c(F)cccc2-c2ccc3ncc(CCN)n3c2)c(CN)nn1C ; Cc1c(CCOc2c(F)cccc2-c2ccc3ncc(CCN)n3c2)c(CNS(C)(=O)=O)nn1C ; Cc1c(CCOc2ccc(F)cc2-c2ccc3ncc(CCN)n3c2)c(C(N)=O)nn1C ; CNC(=O)c1nn(C)c(C)c1CCOc1ccc(F)cc1-c1ccc2ncc(CCN)n2c1 ; Cc1c(CCOc2ccc(F)cc2-c2ccc3ncc(CCN)n3c2)c(C(=O)N(C)C)nn1C]

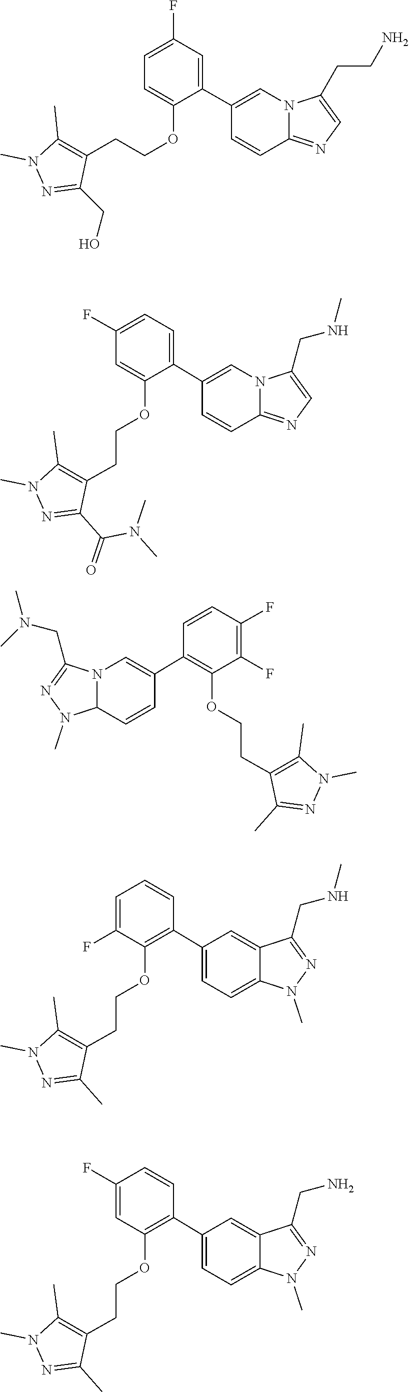

or a pharmaceutically acceptable salt, hydrate or solvate thereof.

In another embodiment, the NMT inhibitor is any one of the following compounds:

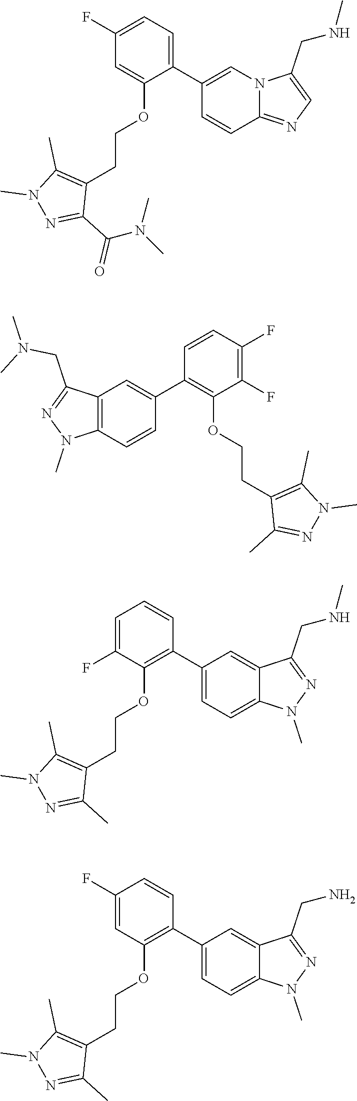

or a pharmaceutically acceptable salt, hydrate or solvate thereof.

In another embodiment, the NMT inhibitor is any one of the following compounds:

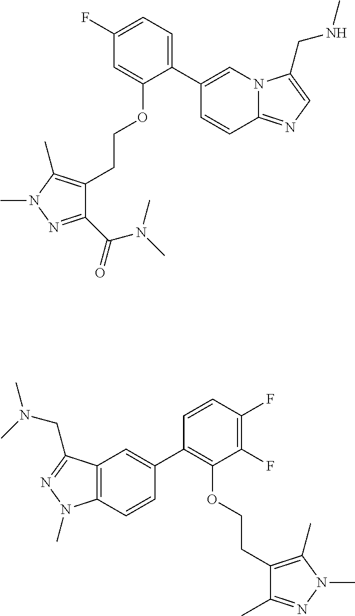

or a pharmaceutically acceptable salt, hydrate or solvate thereof.

In another particular embodiment, the NMT inhibitor is the following compound:

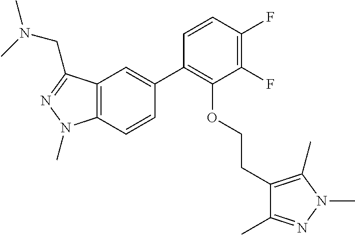

or a pharmaceutically acceptable salt, hydrate or solvate thereof.

In another particular embodiment, the NMT inhibitor is a compound of Formula (Id), shown below, or a pharmaceutically acceptable salt, hydrate or solvate thereof:

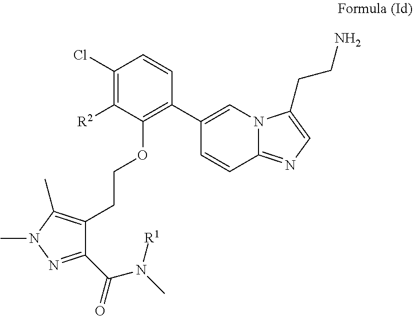

wherein:

R¹ is H or —CH₃; and

R² is H or F.

In another particular embodiment, the NMT inhibitor is selected from any one of the following compounds:

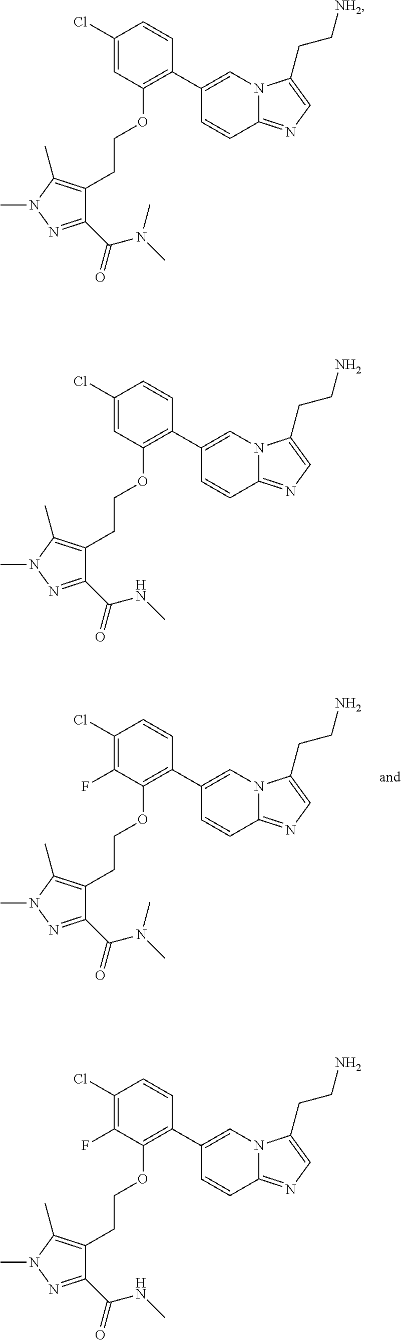 [SMILES: Cc1c(CCOc2cc(Cl)ccc2-c2ccc3ncc(CCN)n3c2)c(C(=O)N(C)C)nn1C ; CNC(=O)c1nn(C)c(C)c1CCOc1cc(Cl)ccc1-c1ccc2ncc(CCN)n2c1 ; Cc1c(CCOc2c(-c3ccc4ncc(CCN)n4c3)ccc(Cl)c2F)c(C(=O)N(C)C)nn1C ; CNC(=O)c1nn(C)c(C)c1CCOc1c(-c2ccc3ncc(CCN)n3c2)ccc(Cl)c1F]

(i.e. 4-(2-{2-[3-(2-aminoethyl)imidazo[1,2-a]pyridin-6-yl]-5-chlorophenoxy}ethyl)-N,N,1,5-tetramethyl-1H-pyrazole-3-carboxamide, 4-(2-{2-[3-(2-aminoethyl)imidazo[1,2-a]pyridin-6-yl]-5-chlorophenoxy}ethyl)-N,1,5-trimethyl-1H-pyrazole-3-carboxamide, 4-(2-{6-[3-(2-aminoethyl)imidazo[1,2-a]pyridin-6-yl]-3-chloro-2-fluorophenoxy}ethyl)-N,N,1,5-tetramethyl-1H-pyrazole-3-carboxamide and 4-(2-{6-[3-(2-aminoethyl)imidazo[1,2-a]pyridin-6-yl]-3-chloro-4-fluorophenoxy}ethyl)-N,1,5-trimethyl-1H-pyrazole-3-carboxamide respectively) or a pharmaceutically acceptable salt, hydrate or solvate thereof.

In another particular embodiment, the NMT inhibitor is selected from any one of the following compounds:

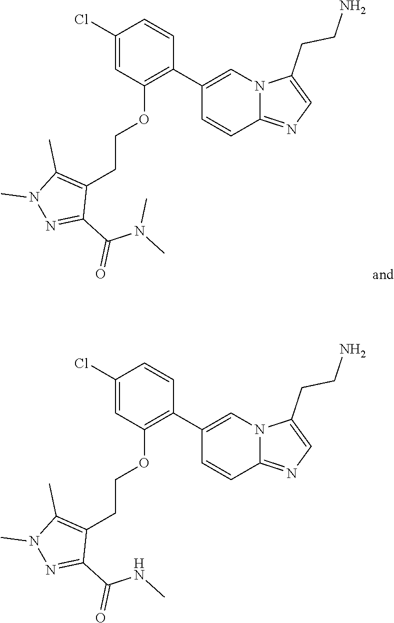 [SMILES: Cc1c(CCOc2cc(Cl)ccc2-c2ccc3ncc(CCN)n3c2)c(C(=O)N(C)C)nn1C ; CNC(=O)c1nn(C)c(C)c1CCOc1cc(Cl)ccc1-c1ccc2ncc(CCN)n2c1]

(i.e. 4-(2-{2-[3-(2-aminoethyl)imidazo[1,2-a]pyridin-6-yl]-5-chlorophenoxy}ethyl)-N,N,1,5-tetramethyl-1H-pyrazole-3-carboxamide and 4-(2-{2-[3-(2-aminoethyl)imidazo[1,2-a]pyridin-6-yl]-5-chlorophenoxy}ethyl)-N,1,5-trimethyl-1H-pyrazole-3-carboxamide) or a pharmaceutically acceptable salt, hydrate or solvate thereof.

In another particular embodiment, the NMT inhibitor is the following compound:

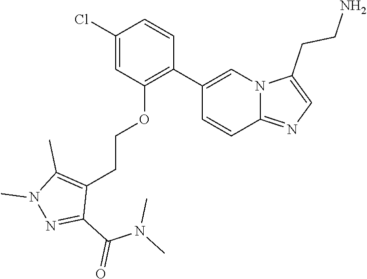 [SMILES: Cc1c(CCOc2cc(Cl)ccc2-c2ccc3ncc(CCN)n3c2)c(C(=O)N(C)C)nn1C]

(i.e. 4-(2-{2-[3-(2-aminoethyl)imidazo[1,2-a]pyridin-6-yl]-5-chlorophenoxy}ethyl)-N,N,1,5-tetramethyl-1H-pyrazole-3-carboxamide) or a pharmaceutically acceptable salt, hydrate or solvate thereof.

### NMT Inhibitors of Formula (II) and Formula (III)

In certain embodiments, the NMT inhibitor is a compound of Formula (II) or Formula (III) shown below, or a pharmaceutically acceptable salt, hydrate or solvate thereof:

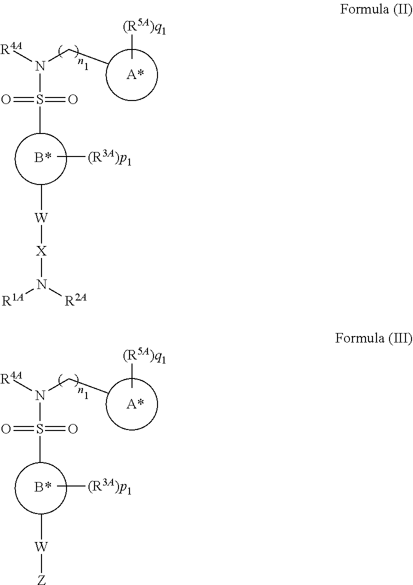

wherein:
m is 0, 1, 2, 3, 4, 5 or 6;
Ring A*, is an optionally substituted nitrogen containing aryl group wherein each substitutable carbon or nitrogen in Ring A* is optionally and independently substituted by one or more R^{5A} and wherein if Ring A* contains an —NH— moiety that nitrogen may be optionally substituted by C₁₋₆alkyl (e.g. methyl); and wherein R^{4A} and Ring A* together with the atoms to which they are attached may form a cyclic group,
Ring B* is an optionally substituted aryl or heteroaryl group wherein each substitutable carbon or heteroatom in Ring B* is optionally and independently substituted by one or more R^{3A};
W and X, one of which may be absent, are independently selected from R^{11A}, hydrocarbyl (e.g. alkyl, alkenyl, alkynyl, or haloalkyl) optionally substituted with R^{11A}, and —(CH₂)ₖ₁-heterocyclyl optionally substituted with R^{12A}; k₁ is 0, 1, 2, 3, 4, 5 or 6;
R^{1A}, R^{2A}, R^{3A}, R^{4A} and R^{5A} are independently selected from hydrogen, R^{12A}, hydrocarbyl (e.g. C₁₋₆ alkyl, alkenyl, alkynyl, or haloalkyl) optionally substituted with R^{12A}, and a —(CH₂)_{L1}— heterocyclyl optionally substituted with one or more R^{12A}; wherein R^{1A} and R^{2A} taken together with the atoms to which they are attached may form a heterocycle, optionally substituted with one or more R^{12A}, wherein R^{1A} and/or R^{2A} taken together with W or X may form a heterocycle optionally substituted with one or more R^{12A}; and wherein one or more of R^{3A} and R^{5A} taken together with the atoms to which they are attached may form a carbocycle, for example heterocyclyl, optionally substituted with R^{12A}; U is 0, 1, 2, 3, 4, 5 or 6;
wherein:
   each R^{11A} and R^{12A} is independently selected from halogen, trifluoromethyl, cyano, thio, nitro, oxo, ═NR^{13A}, —OR^{13A}, —SR^{13A}, —C(O)R^{13A}, —C(O)OR^{13A}, —OC(O)R^{13A}, —NR^{13A}COR^{14A}, —NR^{13A}CON(R^{14A})₂, —NR^{13A}COR^{14A}, —NR^{13A}CO₂R^{14A}, —S(O)R^{13A}, —S(O)₂R^{13A}, —SON(R^{13A})₂, —NR^{13A}S(O)₂R^{14A}; —CSR^{13A}, —N(R^{13A})R^{14A}, —C(O)N(R^{13A})R^{14A}, —SO₂N(R^{13A})R^{14A} and R^{15A};
   R^{13A} and R^{14A} are each independently selected from hydrogen or R^{15A};
   R^{15A} is selected from hydrocarbyl (e.g. C₁₋₆alkyl, alkenyl, alkynyl, or haloalkyl), carbocyclyl and —(CH₂)ₘi-heterocyclyl, and each R^{15A} is optionally and independently substituted with one or more of halogen, cyano, amino, hydroxy, C₁₋₆alkyl or cycloalkyl and C₁₋₆alkoxy;
   m₁ is 0, 1, 2, 3, 4, 5 or 6;
p₁ is 0, 1, 2, 3 or 4; the values of R^{4A} may be the same or different; and
q₁ is 0, 1, 2, 3 or 4; wherein the values of R^{5A} may be the same or different; Y and Z, one or both of which may be absent, are independently selected from hydrogen, R^{16A}, hydrocarbyl (e.g. C₁₋₆alkyl, alkenyl, alkynyl. or haloalkyl) optionally substituted with R^{16A}, and —(CH₂)ᵣi-heterocyclyl optionally substituted with R^{16A}, wherein each R^{16A} is independently selected from halogen, trifluoromethyl, cyano, thio, nitro, oxo, ═NR^{17A}, —OR^{17A}, —SR^{17A}, —C(O)R^{17A}, —C(O)OR^{17A}, —OC(O)R^{17A}, —NR^{17A}COR^{18A}, —NR^{17A}CON(R^{18A})₂, —NR^{17A}COR^{18A}, —NR^{17A}CO₂R^{18A}, —S(O)R^{17A}, —S(O)₂R^{17A}, —SON(R^{17A})₂, —NR^{17A}S(O)₂R^{18A}; —CSR^{17A}, —N(R^{17A})R^{18A}, —C(O)N(R^{17A})R^{18A}, —SO₂N(R^{17A})R^{18A} and R^{19A}; r₁ is 0, 1, 2, 3, 4, 5 or 6;
wherein:
   R^{17A} and R^{18A} are each independently selected from hydrogen or R^{19A};
   R^{19A} is selected from hydrocarbyl (e.g. C₁₋₆alkyl, alkenyl, alkynyl. or haloalkyl), carbocyclyl and —(CH₂)ₛ₁-heterocyclyl, and each R^{19A} is optionally and independently substituted with one or more of halogen, cyano, amino, hydroxy, C₁₋₆alkyl and C₁₋₆alkoxy; and
   s₁ is 0, 1, 2, 3, 4, 5 or 6.

NMT inhibitors of Formula (II) are further described in WO2010/026365. It will be understood that suitable and preferred NMT inhibitors of Formula (II) of the present invention may therefore include any of the compounds disclosed in WO2010/026365.

Suitably, the NMT inhibitor is a compound of Formula (IIa) shown below, or a pharmaceutically acceptable salt, solvate or hydrate thereof:

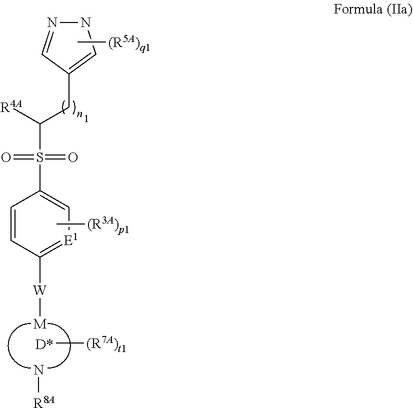

wherein:
m is 0, 1, 2, 3, 4, 5 or 6;
E¹ is independently selected from C and N;
W is selected from a hydrocarbyl optionally substituted with R^{11A}, an optionally substituted aryl or heteroaryl group and a carbocyclyl optionally substituted with one or more of halogen, cyano, amino, hydroxy, C₁₋₆alkyl and C₁₋₆alkoxy groups;
M is selected from C and N;
R^{3A}, R^{4A} and R^{5A} are independently selected from hydrogen, R^{12A}, hydrocarbyl optionally substituted with R^{12A}, and —(CH₂)_{L1}-heterocyclyl optionally substituted with R^{12A};
L₁ is 0, 1, 2, 3, 4, 5 or 6;
wherein each R^{11A} and R^{12A} is independently selected from halogen, trifluoromethyl, cyano, thio, nitro, oxo, ═NR^{13A}, —OR^{13A}, —SR^{13A}, —C(O)R^{13A}, —C(O)OR^{13A}, —OC(O)R^{13A}, —NR^{13A}COR^{14A}, —NR^{13A}CON(R^{13A})₂, —NR^{13A}COR^{14A}, —NR^{13A}CO₂R^{14A}, —S(O)R^{13A}, —S(O)₂R^{13A}, —SON(R^{13A})₂, —NR^{13A}S(O)₂R^{14A}; —CSR^{13A}, —N(R^{13A})R^{14A}, —C(O)N(R^{13A})R^{14A}, —SO₂N(R^{13A})R^{14A} and R^{15A};
R^{13A} and R^{14A} are each independently selected from hydrogen or R^{15A};
wherein R^{15A} is selected from hydrocarbyl, carbocyclyl and —(CH₂)ₘ₁-heterocyclyl, and each R^{15A} is optionally and independently substituted with one or more of halogen, cyano, amino, hydroxy, C₁₋₆alkyl and C₁₋₆alkoxy;
Ring D* is an optionally substituted nitrogen containing 6 or 7 membered heterocycle, wherein each substitutable carbon or nitrogen in Ring D* is optionally and independently substituted by one or more R^{7A};
R^{7A} is independently selected from hydrogen, R^{20A}, hydrocarbyl optionally substituted with R^{20A}, and —(CH₂)_{V1}-heterocyclyl optionally substituted with R^{20A};
v₁ is 0, 1, 2, 3, 4, 5 or 6;
wherein each R^{20A} is independently selected from halogen, trifluoromethyl, cyano, thio, nitro, oxo, ═NR^{21A}, —OR^{21A}, —SR^{21A}, —C(O)R^{21A}, —C(O)OR^{21A}, —OC(O) R^{21A}, —NR^{21A}COR^{22A}, —NR^{21A}CON(R^{22A})₂, —NR^{21A}COR^{22A}, —NR^{21A}CO₂R^{22A}, —S(O)R^{21A}, —S(O)₂R^{21A}, —SON(R^{21A})₂, —NR^{21A}S(O)₂R^{22A}; —CSR^{21A}, N(R^{21A}) R^{22A}, —C(O)N(R^{21A})R^{22A}, —SO₂N(R^{21A})R^{22A} and R^{23A};
wherein R^{21A} and R^{22A} are each independently selected from hydrogen or R^{23A};
wherein R^{23A} is selected from hydrocarbyl, carbocyclyl and —(CH₂)_{w1}-heterocyclyl, and each R^{23A} is optionally and independently substituted with one or more of halogen, cyano, amino, hydroxy, C₁₋₆alkyl and C₁₋₆alkoxy;
w₁ is 0, 1, 2, 3, 4, 5 or 6;
R^{8A} is selected from the list of optional substituents represented by the group R^{4A};
m₁ is 0, 1, 2, 3, 4, 5 or 6;
p¹ is 0, 1, 2, 3 or 4, wherein the values of R^{4A} may be the same or different;
q₁ is 0, 1, 2, 3 or 4, wherein the values of R^{5A} may be the same or different; and
t₁ is 0, 1, 2, 3, 4, 5 or 6, wherein the values of R^{7A} may be the same or different.

More suitably, the NMT inhibitor is a compound of Formula (IIa) shown below, or a pharmaceutically acceptable salt, solvate or hydrate thereof:

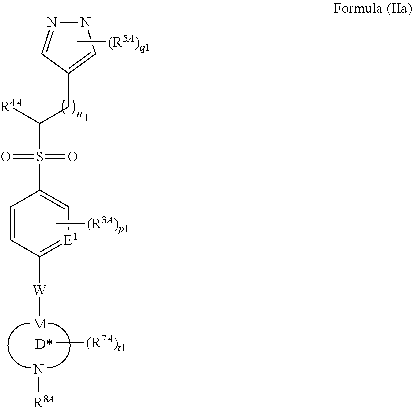

wherein:
n₁ is 0 or 1;
E¹ is C;
W is a (1-4C)hydrocarbyl, an aryl (e.g. phenyl) or heteroaryl group (e.g. pyridinyl);
M is selected from C and N;
R^{3A}, R^{4A} and R^{5A} are independently selected from hydrogen, R^{12A}, and (1-3C)hydrocarbyl optionally substituted with R^{12A};
R^{12A} is independently selected from halogen, trifluoromethyl, cyano, thio, nitro, oxo, —OR^{13A}, —SR^{13A}, —C(O)R^{13A}, —C(O)OR^{13A}, —OC(O)R^{13A}, —NR^{13A}COR^{14A} and R^{15A};
R^{13A} and R^{14A} are each independently selected from hydrogen or a (1-4C)hydrocarbyl (e.g. methyl);
Ring D* is an optionally substituted nitrogen containing 6 or 7 membered heterocycle, wherein each substitutable carbon or nitrogen in Ring D* is optionally and independently substituted by one or more R^{7A};
R^{7A} is independently selected from hydrogen, (1-4C)hydrocarbyl, halogen, trifluoromethyl, cyano, thio, nitro or oxo;
R^{8A} is a hydrogen or a (1-4C)hydrocarbyl (e.g. methyl);
p₁ is 0, 1 or 2, wherein the values of R^{4A} may be the same or different;
q₁ is 3, wherein the values of R^{5A} may be the same or different; and
t₁ is 0, 1 or 2, wherein the values of R^{7A} may be the same or different.

In a particular embodiment, the NMT inhibitor is any one of the compounds shown in Table 1 of WO2010/026365.

In a further embodiment, the NMT inhibitor is a compound selected from:

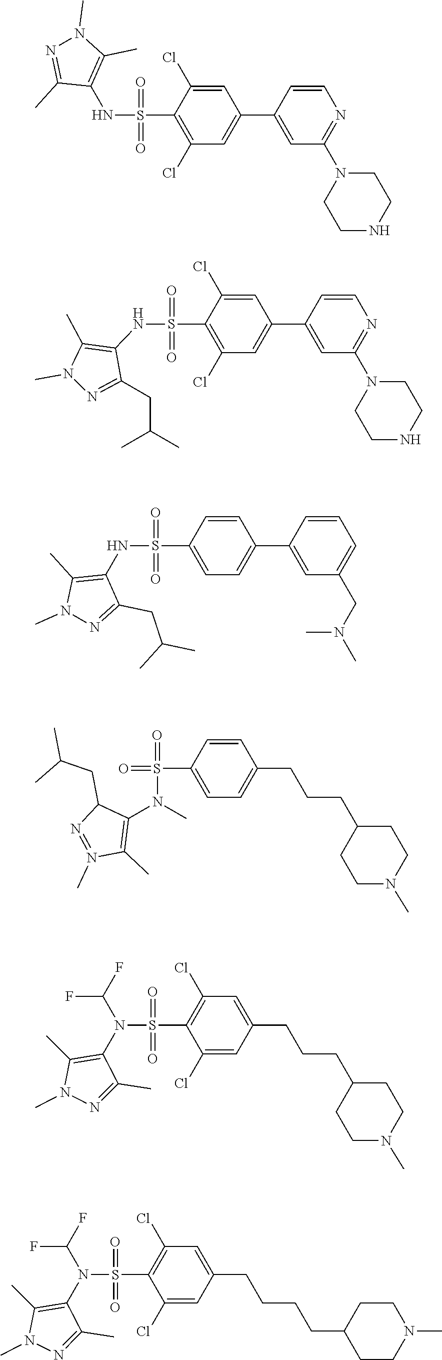

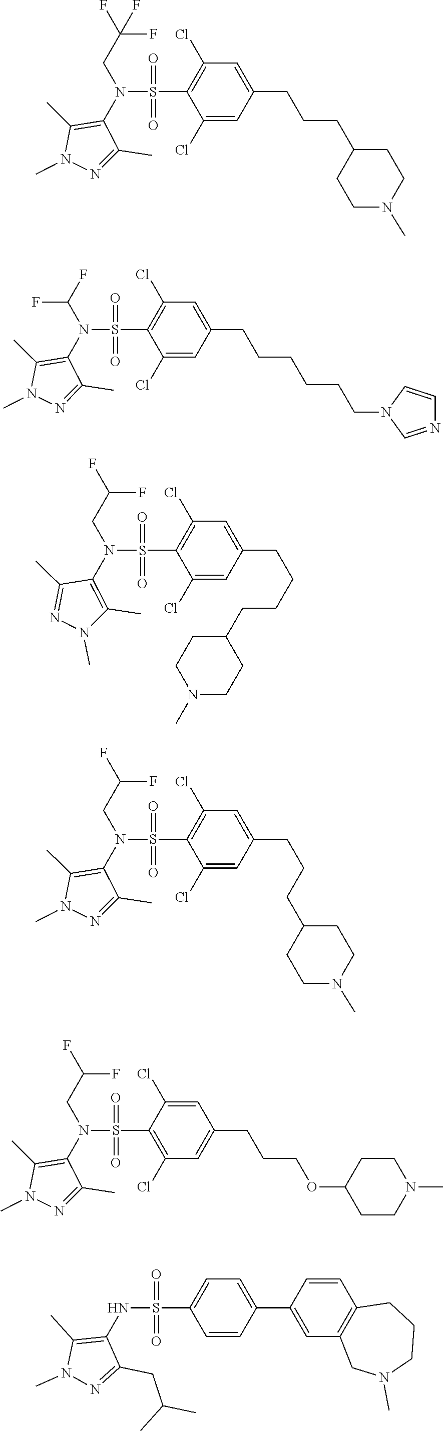

or a pharmaceutically acceptable salt, solvate or hydrate thereof.

In a further embodiment, the NMT inhibitor is a compound selected from:

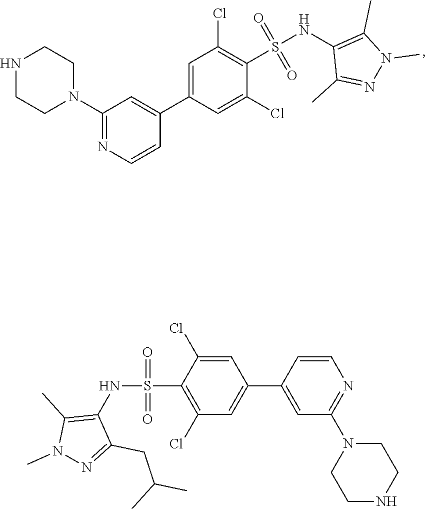

or a pharmaceutically acceptable salt, solvate or hydrate thereof.

In yet a further embodiment, the NMT inhibitor is:

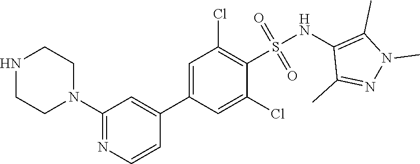 [SMILES: Cc1nn(C)c(C)c1NS(=O)(=O)c1c(Cl)cc(-c2ccnc(N3CCNCC3)c2)cc1Cl]

or a pharmaceutically acceptable salt, solvate or hydrate thereof.

### Other Possible NMT Inhibitors

Other suitable NMT inhibitors may include, for example, any one of the compounds displaying activity as an inhibitor of N-myristoyl transferase (NMT) described in WO00/37464 (Roche) or WO2013/083991 (Imperial Innovations Limited).

In certain embodiments, the NMT inhibitor is any one of the compounds displaying activity as an inhibitor of N-myristoyl transferase (NMT) described in WO2013/083991. It will be appreciated that this embodiment encompasses both generic and specific compounds described in WO2013/083991.

In a particular embodiment, the NMT inhibitor of the present invention is any one of compounds 1-140 disclosed in WO2013/083991.

Salts of the NMT inhibitors for use in the invention are those wherein a counter-ion is pharmaceutically acceptable. Suitable pharmaceutically acceptable salts according to the invention include those formed with organic or inorganic acids or bases. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄)-alkyl- or aryl-sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. For example, it may be the hydrochloric (HCl) salt.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The NMT inhibitor may possess one or more asymmetric centers; such compounds can therefore be produced as individual (R)- or (S)-stereoisomers or as mixtures thereof. It is to be understood that the NMT inhibitors for use in the present invention encompasses all optical, diastereoisomers and geometric isomers and mixtures thereof that possess NMT inhibition activity.

The present invention also encompasses NMT inhibitors as defined herein which comprise one or more isotopic substitutions. For example, H may be in any isotopic form, including 1H, 2H(D), and 3H (T); C may be in any isotopic form, including 12C, 13C, and 14C; and O may be in any isotopic form, including 160 and 180; and the like.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates, such as hydrates, exist when the drug substance incorporates solvent, such as water, in the crystal lattice in either stoichiometric or non-stoichiometric amounts. Drug substances are routinely screened for the existence of hydrates since these may be encountered at any stage of the drug manufacturing process or upon storage of the drug substance or dosage form. Solvates are described in S. Byrn et al., Pharmaceutical Research, 1995. 12(7): p. 954-954, and Water-Insoluble Drug Formulation, 2^{nd} ed. R. Liu, CRC Press, page 553, which are incorporated herein by reference. Accordingly, it will be understood by the skilled person that the NMT inhibitors for use in the present invention may be present in the form of solvates, wherein the associated solvent is a pharmaceutically acceptable solvent. For example, a hydrate is an example of a pharmaceutically acceptable solvate.

It is also to be understood that the NMT inhibitors may exhibit polymorphism, and that the invention encompasses all such forms that possess the herein described activity.

The NMT inhibitors for use in the present invention may also exist in a number of different tautomeric forms and references to NMT inhibitors for use in the present invention include all such forms. For the avoidance of doubt, where a compound can exist in one of several tautomeric forms, and only one is specifically described or shown, all others are nevertheless embraced. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

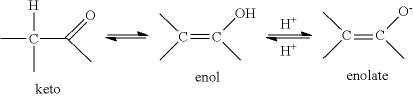

The NMT inhibitor for use in the present invention may be administered in the form of a pro-drug which is broken down in the human or animal body to release the NMT inhibitor for use in the present invention. A pro-drug may be used to alter the physical properties and/or the pharmacokinetic properties of the NMT inhibitor. A pro-drug can be formed when the NMT inhibitor contains a suitable group or substituent to which a property-modifying group can be attached. Examples of pro-drugs include in vivo cleavable ester derivatives that may be formed at a carboxy group or a hydroxy group in an NMT inhibitor for use in the present invention, and in-vivo cleavable amide derivatives that may be formed at a carboxy group or an amino group in an NMT inhibitor for use in the present invention.

For example, the NMT inhibitors for use in the present invention may have an appropriate group which may be converted to an amide or a carbamate. Typical amide and carbamate groups formed from a basic nitrogen in the NMT inhibitors of the present invention, for example, the compounds of Formula (I), include >NR^{G}C(O)R^{G}, >NR^{G}CO₂R^{G}, and >NR^{G}SO₂R^{G}, where R^{G} is selected from the group consisting of C₁₋₆alkyl, C₂₋₈alkenyl, C₂₋₈alkynyl, C₃₋₈cycloalkyl and C₃₋₈cycloalkylC₁₋₈alkyl, haloC₁₋₈alkyl, dihaloC₁₋₈alkyl, trihaloC₁₋₈alkyl, phenyl and phenylC₁₋₄alkyl; more preferably R^{G} is selected from the group consisting of C₁₋₆alkyl, C₂₋₆alkenyl, C₂₋₆alkynyl, C₃₋₈cycloalkyl and C₃₋₈cycloalkylC₁₋₈alkyl

### Particularly Preferred Embodiments

Particularly preferred embodiments of the present invention are set out hereinbelow in numbered paragraphs 1.1 to 1.12.

### Treatment of Cancer Comprising One or More Structural Alterations of the MYC Locus

1.1 A NMT inhibitor, or a pharmaceutically acceptable salt, solvate or hydrate thereof, for use in the treatment of a cancer comprising one or more structural alterations of the MYC locus,
   wherein:
      i) the one or more structural alterations are selected from mutations, copy-number gains and/or chromosomal rearrangements;
      ii) the cancer is selected from high grade mantle zone lymphoma, follicular lymphoma, plasmablastic lymphoma, diffuse large B-cell lymphoma, Burkitt's lymphoma, multiple myeloma, blastoma (e.g. a neuroblastoma, retinoblastoma or glioblastoma), chronic lymphocytic leukaemia, acute myeloid leukemia, B-acute lymphocytic leukaemia and a solid tumour in an organ selected from the breast, colon and gallbladder; and
      iii) the NMT inhibitor is a compound of Formula I (e.g. Formula IA\hat{ }\hat{ }) or Formula II (e.g. Formula IIa) described herein.
1.2 A NMT inhibitor, or a pharmaceutically acceptable salt, solvate or hydrate thereof, for use in the treatment of a cancer comprising one or more structural alterations of the MYC locus,
   wherein:
      i) the one or more structural alterations are mutations;
      ii) the cancer is selected from high grade mantle zone lymphoma, follicular lymphoma, plasmablastic lymphoma, diffuse large B-cell lymphoma, Burkitt's lymphoma, multiple myeloma, blastoma (e.g. a neuroblastoma, retinoblastoma or glioblastoma), chronic lymphocytic leukaemia, acute myeloid leukemia, B-acute lymphocytic leukaemia and a solid tumour in an organ selected from the breast, colon and gallbladder; and; and
      iii) the NMT inhibitor is a compound of Formula I, preferably Formula (IA\hat{ }\hat{ }), (e.g. Compound 1, Compound 2, Compound 3 or Compound 4) described herein.
1.3 A NMT inhibitor, or a pharmaceutically acceptable salt, solvate or hydrate thereof, for use in the treatment of a cancer comprising one or more structural alterations of the MYC locus (e.g. one or more structural alterations of the c-MYC and/or MYCN locus),
   wherein:
      i) the one or more structural alterations are mutations;
      i) the cancer is selected from diffuse large B-cell lymphoma, Burkitt's lymphoma, multiple myeloma, neuroblastoma, retinoblastoma, glioblastoma, acute myeloid leukemia, B-acute lymphocytic leukaemia and a solid tumour in an organ selected from the breast, colon and gallbladder; and
      ii) the NMT inhibitor is a compound of Formula (IA\hat{ }\hat{ }) (e.g. Compound 1, Compound 2, Compound 3 or Compound 4) shown below, or a pharmaceutically acceptable salt, solvate or hydrate thereof:

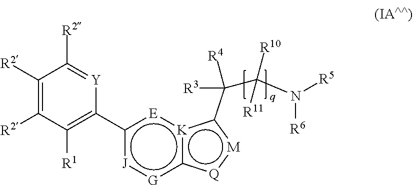

wherein:
   R¹ is a group of formula —X-L-A;
   A is 4-pyrazolyl, said pyrazolyl being optionally substituted with up to 3 methyl groups;
   X is —O— or absent;
   L is —(CH₂)ₘ— or —(CH₂)ₘ—O—;
   m is 2;
   R^{2′} is selected from the group consisting of fluorine, chlorine —CN and methyl (preferably fluorine);
   R^{2″} is selected from the group consisting of hydrogen, fluorine, chlorine, —CN and methyl;
   q is 0 or 1;
   R³ is hydrogen or methyl;
   R⁴ is hydrogen or methyl;
   R⁵ is hydrogen or methyl;
   R⁶ is hydrogen or methyl; or
   the R³ group and the R⁵ group and the intervening atoms form a 3 to 7 membered non-aromatic heterocycle composed of the intervening atoms and bonds, (more preferably R⁵ and R⁶ are both methyl);
   R⁷ where present is hydrogen or methyl;
   R⁸ where present is hydrogen or methyl;
   R⁹ and R¹⁰ where present are hydrogen or methyl; and
   E, J, G, K, Q and M are:
   i) E, J and G are each C(R⁷), K is carbon, Q is N(R⁸), M is nitrogen; and R⁸ is hydrogen or methyl;
   ii) E, J and G are each C(R⁷), and K, Q and M are each nitrogen;
   iii) E and G are each C(R⁷), and J, K, Q and M are each nitrogen;
   iv) J and G are each C(R⁷), and E, K, Q and M are each nitrogen; or
   v) E, J, G and M are each C(R⁷), and K and Q are each nitrogen.
1.4 A NMT inhibitor, or a pharmaceutically acceptable salt, solvate or hydrate thereof, for use in the treatment of a cancer comprising one or more structural alterations of the MYC locus (e.g. one or more structural alterations of the c-MYC and/or MYCN locus),
   wherein:
      i) the one or more structural alterations are mutations which impart overexpression of MYC (e.g. c-MYC or MYCN);
      i) the cancer is selected from diffuse large B-cell lymphoma, a neuroblastoma, retinoblastoma, a glioblastoma, acute myeloid leukemia, B-acute lymphocytic leukaemia and breast cancer (e.g. triple negative breast cancer); and
      ii) the NMT inhibitor is a compound of Formula (IA\hat{ }\hat{ }) (e.g. Compound 1, Compound 2, Compound 3 or Compound 4) shown below, or a pharmaceutically acceptable salt, solvate or hydrate thereof:

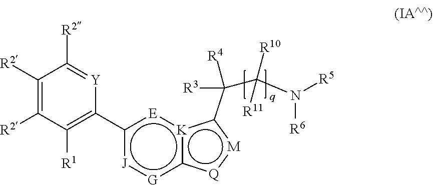

wherein:
   R¹ is a group of formula —X-L-A;
   A is 4-pyrazolyl, said pyrazolyl being optionally substituted with up to 3 substituent groups selected from C₁₋₄alkyl (e.g. methyl) and —C(O)N(R⁹)₂ (e.g. —C(O)N(CH₃)₂);
   X is —O— or absent;
   L is —(CH₂)ₘ— or —(CH₂)ₘ—O—;
   m is 2;
   R^{2′} is selected from the group consisting of fluorine or chlorine (preferably fluorine);
   R^{2″} is selected from the group consisting of hydrogen, fluorine or chlorine;
   q is 0 or 1;
   R³ is hydrogen or methyl;
   R⁴ is hydrogen or methyl;
   R⁵ is hydrogen or methyl;
   R⁶ is hydrogen or methyl; or
   the R³ group and the R⁵ group and the intervening atoms form a 3 to 7 membered non-aromatic heterocycle composed of the intervening atoms and bonds, (more preferably R⁵ and R⁶ are both methyl);
   R⁷ where present is hydrogen or methyl;
   R⁸ where present is hydrogen or methyl;
   R⁹ and R¹⁰ where present are hydrogen or methyl; and
   E, J, G, K, Q and M are:
   i) E, J and G are each C(R⁷), K is carbon, Q is N(R⁸), M is nitrogen; and R⁸ is hydrogen or methyl; or
   ii) E, J, G and M are each C(R⁷), and K and Q are each nitrogen;
with the proviso that A is substituted with no more than one —C(O)N(R⁹)₂ group.
1.5 A NMT inhibitor, or a pharmaceutically acceptable salt, solvate or hydrate thereof, for use in the treatment of a acceptable salt, solvate or hydrate thereof, for use in the treatment of a cancer comprising one or more structural alterations of the MYC locus (e.g. one or more structural alterations of the c-MYC and/or MYCN locus),
   wherein:
      i) the one or more structural alterations are mutations which impart overexpression of MYC (e.g. c-MYC or MYCN);
      ii) the cancer is selected from diffuse large B-cell lymphoma, a neuroblastoma, retinoblastoma, glioblastoma, acute myeloid leukemia, B-acute lymphocytic leukaemia and breast cancer (e.g. triple negative breast cancer); and
      iii) the NMT inhibitor is a compound of Formula (IA\hat{ }\hat{ }) (e.g. Compound 1 or Compound 2) shown below, or a pharmaceutically acceptable salt, solvate or hydrate thereof:

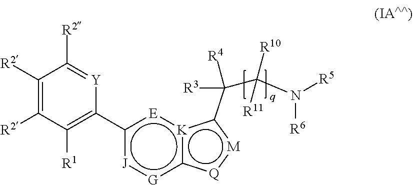

wherein:
   R¹ is a group of formula —X-L-A;
   A is 4-pyrazolyl, said pyrazolyl being optionally substituted with up to 3 substituent groups selected from methyl and —C(O)N(CH₃)₂;
   X is —O— or absent;
   L is —(CH₂)ₘ— or —(CH₂)ₘ—O—;
   m is 2;
   R^{2′} is selected from the group consisting of fluorine or chlorine (preferably fluorine);
   R^{2″} is selected from the group consisting of hydrogen, fluorine or chlorine;
   q is 0;
   R³ is hydrogen or methyl;
   R⁴ is hydrogen or methyl;
   R⁵ is hydrogen or methyl;
   R⁶ is hydrogen or methyl; or
   the R³ group and the R⁵ group and the intervening atoms form a 3 to 7 membered non-aromatic heterocycle composed of the intervening atoms and bonds, (more preferably R⁵ and R⁶ are both methyl);
   E, J, G, K, Q and M are:
   i) E, J and G are each CH, K is carbon, Q is N(R⁸), M is nitrogen; and R⁸ is hydrogen or methyl; or
   ii) E, J, G and M are each CH, and K and Q are each nitrogen;
with the proviso that A is substituted with no more than one —C(O)N(CH₃)₂ group.
1.6 A NMT inhibitor, or a pharmaceutically acceptable salt, solvate or hydrate thereof, for use in the treatment of a acceptable salt, solvate or hydrate thereof, for use in the treatment of a cancer comprising one or more structural alterations of the MYC locus (e.g. one or more structural alterations of the c-MYC and/or MYCN locus),
   wherein:
      i) the one or more structural alterations are mutations which impart overexpression of MYC (e.g. c-MYC or MYCN), preferably, such that the levels of RNA transcript and/or protein of MYC (e.g. c-MYC or MYCN) are at least 25% greater than the RNA transcript and/or protein levels of MYC (e.g. c-MYC or MYCN) found in a normal, healthy cell;
      ii) the cancer is selected from diffuse large B-cell lymphoma, a neuroblastoma, acute myeloid leukemia, B-acute lymphocytic leukaemia and breast cancer (e.g. triple negative breast cancer, such as basal-like breast cancer, or breast invasive carcinoma); and
      iii) the NMT inhibitor is a compound of Formula (IA\hat{ }\hat{ }) (e.g. Compound 1 or Compound 2) shown below, or a pharmaceutically acceptable salt, solvate or hydrate thereof:

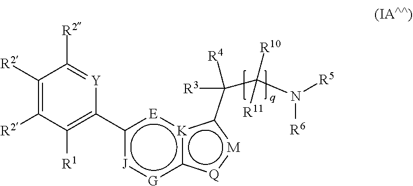

wherein:
   R¹ is a group of formula —X-L-A;
   A is 4-pyrazolyl, said pyrazolyl being optionally substituted with up to 3 substituent groups selected from methyl and —C(O)N(CH₃)₂;
   X is —O— or absent;
   L is —(CH₂)ₘ— or —(CH₂)ₘ—O—;
   m is 2;
   R^{2′} is selected from the group consisting of fluorine or chlorine (preferably fluorine);
   R^{2″} is selected from the group consisting of hydrogen, fluorine or chlorine;
   q is 0;
   R³ is hydrogen or methyl;
   R⁴ is hydrogen or methyl;
   R⁵ is hydrogen or methyl;
   R⁶ is hydrogen or methyl; or
   the R³ group and the R⁵ group and the intervening atoms form a 3 to 7 membered non-aromatic heterocycle composed of the intervening atoms and bonds, (more preferably R⁵ and R⁶ are both methyl);
   E, J, G, K, Q and M are:
   i) E, J and G are each CH, K is carbon, Q is N(R⁸), M is nitrogen; and R⁸ is hydrogen or methyl; or
   ii) E, J, G and M are each CH, and K and Q are each nitrogen;
with the proviso that A is substituted with no more than one —C(O)N(CH₃)₂ group.

### Treatment of MYC Addicted Cancers

1.7 A NMT inhibitor, or a pharmaceutically acceptable salt, solvate or hydrate thereof, for use in the treatment of a MYC addicted cancer,
   wherein:
      i) MYC is overexpressed in said MYC addicted cancer;
      ii) the MYC addicted cancer is selected from high grade mantle zone lymphoma, follicular lymphoma, plasmablastic lymphoma, diffuse large B-cell lymphoma, Burkitt's lymphoma, multiple myeloma, blastoma (e.g. a neuroblastoma, retinoblastoma or glioblastoma), chronic lymphocytic leukaemia, acute myeloid leukemia, B-acute lymphocytic leukaemia and a solid tumour in an organ selected from the breast, colon and gallbladder; and
      iii) the NMT inhibitor is a compound of Formula I (e.g. Formula IA\hat{ }\hat{ }) or Formula II (e.g. Formula IIa) described herein.
1.8 A NMT inhibitor, or a pharmaceutically acceptable salt, solvate or hydrate thereof, for use in the treatment of a MYC addicted cancer (e.g. a c-MYC addicted cancer or a MYCN addicted cancer):
   wherein:
      i) MYC is overexpressed in said MYC addicted cancer;
      ii) the MYC addicted cancer is selected from diffuse large B-cell lymphoma, Burkitt's lymphoma, multiple myeloma, blastoma (e.g. a neuroblastoma, retinoblastoma or glioblastoma), acute myeloid leukemia, B-acute lymphocytic leukaemia and a solid tumour in an organ selected from the breast, colon and gallbladder; and
      iii) the NMT inhibitor is a compound of Formula I, preferably Formula (IA\hat{ }\hat{ }), (e.g. Compound 1, Compound 2, Compound 3 or Compound 4) described herein.
1.9 A NMT inhibitor, or a pharmaceutically acceptable salt, solvate or hydrate thereof, for use in the treatment of a MYC addicted cancer (e.g. a c-MYC addicted cancer or a MYCN addicted cancer):
   wherein:
      i) MYC is overexpressed in said MYC addicted cancer, preferably, such that the levels of RNA transcript and/or protein of MYC in the MYC addicted cancer are at least 25% greater than the RNA transcript and/or protein levels of MYC found in a normal, healthy cell;
      ii) the MYC addicted cancer is selected from diffuse large B-cell lymphoma, Burkitt's lymphoma, multiple myeloma, a neuroblastoma, retinoblastoma, glioblastoma, acute myeloid leukemia, B-acute lymphocytic leukaemia and a solid tumour in an organ selected from the breast, colon and gallbladder; and
      iii) the NMT inhibitor is a compound of Formula I, preferably Formula (IA\hat{ }\hat{ }), (e.g. Compound 1, Compound 2, Compound 3 or Compound 4) described herein.
1.10 A NMT inhibitor, or a pharmaceutically acceptable salt, solvate or hydrate thereof, for use in the treatment of MYC addicted cancer (e.g. a c-MYC addicted cancer or a MYCN addicted cancer);
   wherein:
      i) MYC is overexpressed in said MYC addicted cancer, preferably, such that the levels of RNA transcript and/or protein of MYC in the MYC addicted cancer are at least 25% greater than the RNA transcript and/or protein levels of MYC found in a normal, healthy cell;
      ii) the MYC addicted cancer is selected from diffuse large B-cell lymphoma, a Burkitt's lymphoma, multiple myeloma, a neuroblastoma, retinoblastoma, glioblastoma, acute myeloid leukemia, B-acute lymphocytic leukaemia and a solid tumour in an organ selected from the breast, colon and gallbladder; and
      iii) the NMT inhibitor is a compound of Formula (IA\hat{ }\hat{ }) (e.g. Compound 1, Compound 2, Compound 3 or Compound 4) shown below, or a pharmaceutically acceptable salt, solvate or hydrate thereof:

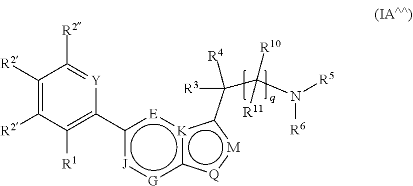

wherein:
   R¹ is a group of formula —X-L-A;
   A is 4-pyrazolyl, said pyrazolyl being optionally substituted with up to 3 substituent groups selected from C₁₋₄alkyl, wherein each —C₁₋₄alkyl is optionally substituted by up to 3 halogen, hydroxyl or —OC₁₋₄alkyl groups; —C(O)N(R⁹)₂ (for example —C(O)N(H)C₁₋₄alkyl), and —CH—₂C(O)N(R⁹)₂ (for example —CH₂C(O)N(H)C₁₋₄alkyl);
   X is —O— or absent;
   L is —(CH₂)ₘ— or —(CH₂)ₘ—O—;
   m is 2;
   R^{2′} is selected from the group consisting of fluorine, chlorine —CN and methyl (preferably fluorine);
   R^{2″} is selected from the group consisting of hydrogen, fluorine, chlorine, —CN and methyl;
   q is 0 or 1;
   R³ is hydrogen or methyl;
   R⁴ is hydrogen or methyl;
   R⁵ is hydrogen or methyl;
   R⁶ is hydrogen or methyl; or
   the R³ group and the R⁵ group and the intervening atoms form a 3 to 7 membered non-aromatic heterocycle composed of the intervening atoms and bonds, (more preferably R⁵ and R⁶ are both methyl);
   R⁷ where present is hydrogen or methyl;
   R⁸ where present is hydrogen or methyl;
   R⁹ and R¹⁰ where present are hydrogen or methyl; and
   E, J, G, K, Q and M are:
   i) E, J and G are each C(R⁷), K is carbon, Q is N(R⁸), M is nitrogen; and R⁸ is hydrogen or methyl;
   ii) E, J and G are each C(R⁷), and K, Q and M are each nitrogen;
   iii) E and G are each C(R⁷), and J, K, Q and M are each nitrogen;
   iv) J and G are each C(R⁷), and E, K, Q and M are each nitrogen; or
   v) E, J, G and M are each C(R⁷), and K and Q are each nitrogen.
1.11 A NMT inhibitor, or a pharmaceutically acceptable salt, solvate or hydrate thereof, for use in the treatment of a MYC addicted cancer (e.g. a c-MYC addicted cancer or a MYCN addicted cancer);
   wherein:
      i) MYC is overexpressed in said transcriptionally addicted cancer, preferably, such that the levels of RNA transcript and/or protein of MYC in the MYC addicted cancer are at least 25% greater than the RNA transcript and/or protein levels of MYC found in a normal, healthy cell;
      ii) the MYC addicted cancer is selected from diffuse large B-cell lymphoma, a neuroblastoma, retinoblastoma, glioblastoma, acute myeloid leukemia, B-acute lymphocytic leukaemia and breast cancer (e.g. triple negative breast cancer); and
      iii) the NMT inhibitor is a compound of Formula (IA\hat{ }\hat{ }) (e.g. Compound 1, Compound 2, Compound 3 or Compound 4) shown below, or a pharmaceutically acceptable salt, solvate or hydrate thereof:

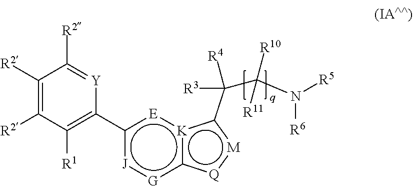

wherein:
   R¹ is a group of formula —X-L-A;
   A is 4-pyrazolyl, said pyrazolyl being optionally substituted with up to 3 substituent groups selected from C₁₋₄alkyl (e.g. methyl) and —C(O)N(R⁹)₂ (e.g. —C(O)N(CH₃)₂);
   X is —O— or absent;
   L is —(CH₂)ₘ— or —(CH₂)ₘ—O—;
   m is 2;
   R^{2′} is selected from the group consisting of fluorine or chlorine (preferably fluorine);
   R^{2″} is selected from the group consisting of hydrogen, fluorine or chlorine;
   q is 0 or 1;
   R³ is hydrogen or methyl;
   R⁴ is hydrogen or methyl;
   R⁵ is hydrogen or methyl;
   R⁶ is hydrogen or methyl; or
   the R³ group and the R⁵ group and the intervening atoms form a 3 to 7 membered non-aromatic heterocycle composed of the intervening atoms and bonds, (more preferably R⁵ and R⁶ are both methyl);
   R⁷ where present is hydrogen or methyl;
   R⁸ where present is hydrogen or methyl;
   R⁹ and R¹⁰ where present are hydrogen or methyl; and
   E, J, G, K, Q and M are:
   i) E, J and G are each C(R⁷), K is carbon, Q is N(R⁸), M is nitrogen; and R⁸ is hydrogen or methyl; or
   ii) E, J, G and M are each C(R⁷), and K and Q are each nitrogen;
with the proviso that A is substituted with no more than one —C(O)N(R⁹)₂ group.
1.12 A NMT inhibitor, or a pharmaceutically acceptable salt, solvate or hydrate thereof, for use in the treatment of a MYC addicted cancer (e.g. a c-MYC addicted cancer or a MYCN addicted cancer);
   wherein:
      i) MYC is overexpressed in said MYC addicted cancer, preferably, such that the levels of RNA transcript and/or protein of MYC in the MYC addicted cancer are at least 25% greater than the RNA transcript and/or protein levels of MYC found in a normal, healthy cell;
      ii) the MYC addicted cancer is selected from diffuse large B-cell lymphoma, neuroblastoma, retinoblastoma, glioblastoma, small cell lung carcinoma, astrocytoma, multiple myeloid leukaemia, B-acute lymphocytic leukaemia, triple negative breast cancer (e.g. basal-like breast cancer) and breast invasive carcinoma; and
      iii) the NMT inhibitor is a compound of Formula (IA\hat{ }\hat{ }) (e.g. Compound 1 or Compound 2) shown below, or a pharmaceutically acceptable salt, solvate or hydrate thereof:

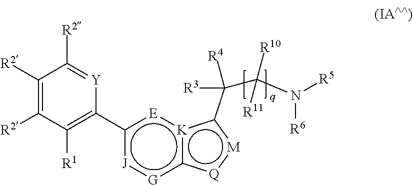

wherein:
   R¹ is a group of formula —X-L-A;
   A is 4-pyrazolyl, said pyrazolyl being optionally substituted with up to 3 substituent groups selected from methyl and —C(O)N(CH₃)₂;
   X is —O— or absent;
   L is —(CH₂)ₘ— or —(CH₂)ₘ—O—;
   m is 2;
   R^{2′} is selected from the group consisting of fluorine or chlorine (preferably fluorine);
   R^{2″} is selected from the group consisting of hydrogen, fluorine or chlorine;
   q is 0;
   R³ is hydrogen or methyl;
   R⁴ is hydrogen or methyl;
   R⁵ is hydrogen or methyl;
   R⁶ is hydrogen or methyl; or
   the R³ group and the R⁵ group and the intervening atoms form a 3 to 7 membered non-aromatic heterocycle composed of the intervening atoms and bonds, (more preferably R⁵ and R⁶ are both methyl);
   E, J, G, K, Q and M are:
   i) E, J and G are each CH, K is carbon, Q is N(R⁸), M is nitrogen; and R⁸ is hydrogen or methyl; or
   ii) E, J, G and M are each CH, and K and Q are each nitrogen;
with the proviso that A is substituted with no more than one —C(O)N(CH₃)₂ group.

### Synthesis

The NMT inhibitors for use in the present invention can be prepared by any suitable technique known in the art. Particular processes for the preparation of the NMT inhibitors described herein may be found in WO00/37464 (Roche), WO2010/026365 (University of Dundee), WO2013/083991 (Imperial Innovations Limited) and WO2017/001812 (Imperial Innovations Limited).

In the description of the synthetic methods described herein and in any of the references noted above, it is to be understood that all proposed reaction conditions, including choice of solvent, reaction atmosphere, reaction temperature, duration of the experiment and workup procedures, can be selected by a person skilled in the art.

Numerous synthetic routes to the NMT inhibitors described herein can be devised by a person skilled in the art and the exemplified synthetic routes described in the above references do not limit the invention. Many methods exist in the literature for the synthesis of heterocycles, for example: Joule, J. AC Mills, K., Heterocyclic Chemistry, 2010, 5^{th} Edition, Pub. Wiley.

### Examples

### NMT Inhibitors

Throughout the accompanying Examples section, and throughout the specification as a whole, reference to Compounds 1, 2, 3, 4, 5, 6 and/or 7 will be understood to be a reference to the compound(s) shown below:

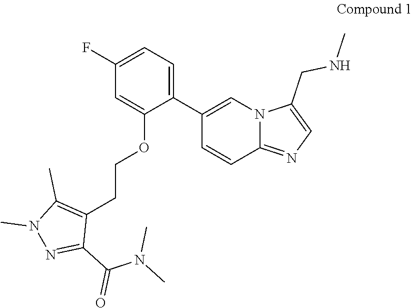

### (4-[2-(5-fluoro-2-{3-[(methylamino)methyl]imidazo[1,2-a]pyridin-6-yl}phenoxy)ethyl]-N,N,1,5-tetramethyl-1H-pyrazole-3-carboxamide)

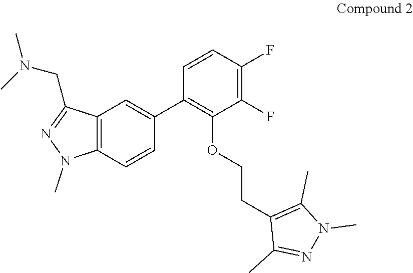

### (1-(5-(3,4-difluoro-2-(2-(1,3,5-trimethyl-1H-pyrazol-4-yl)ethoxy)phenyl)-1-methyl-1H-indazol-3-yl)-N,N-dimethylmethanamine)

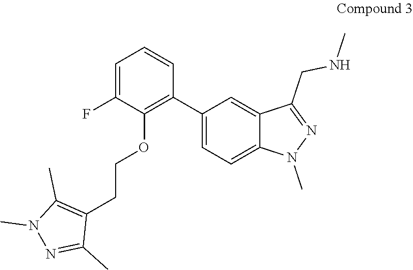

### (1-(5-(3-fluoro-2-(2-(1,3,5-trimethyl-1H-pyrazol-4-yl)ethoxy)phenyl)-1-methyl-1H-indazol-3-yl)-N-methylmethanamine)

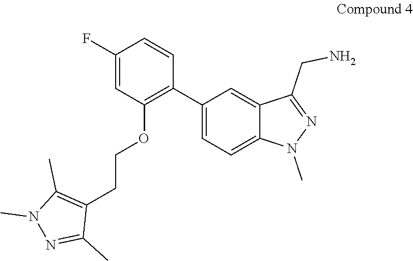 [SMILES: Cc1nn(C)c(C)c1CCOc1cc(F)ccc1-c1ccc2c(c1)c(CN)nn2C]

### ((5-(4-fluoro-2-(2-(1,3,5-trimethyl-1H-pyrazol-4-yl)ethoxy)phenyl)-1-methyl-1H-indazoyl-3-yl)methanamine)

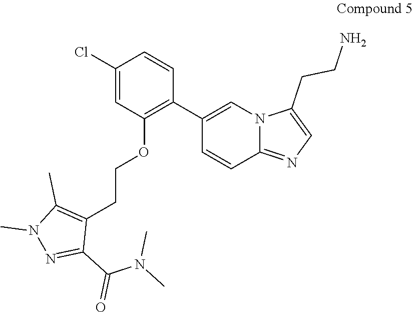 [SMILES: Cc1c(CCOc2cc(Cl)ccc2-c2ccc3ncc(CCN)n3c2)c(C(=O)N(C)C)nn1C]

### (4-(2-{2-[3-(2-aminoethyl)imidazo[1,2-a]pyridin-6-yl]-5-chlorophenoxy}ethyl)-N,N,1,5-tetramethyl-1H-pyrazole-3-carboxamide)

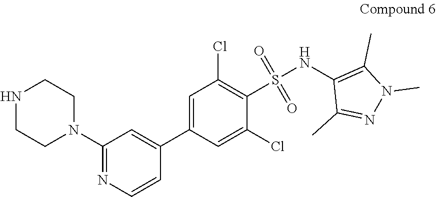 [SMILES: Cc1nn(C)c(C)c1NS(=O)(=O)c1c(Cl)cc(-c2ccnc(N3CCNCC3)c2)cc1Cl]

### (2,6-Dichloro-4-(2-piperazin-1-yl-pyridin-4-yl)-N-(1,3,5-trimethyl-1H-pyraxol-4-yl)-benzenesulfonamide)

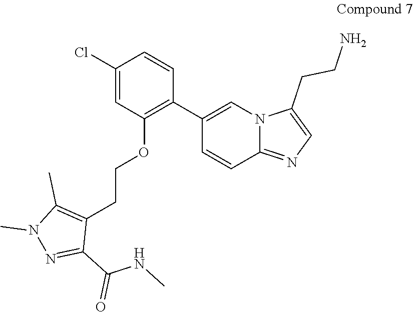 [SMILES: CNC(=O)c1nn(C)c(C)c1CCOc1cc(Cl)ccc1-c1ccc2ncc(CCN)n2c1]

### (4-(2-{2-[3-(2-aminoethyl)imidazo[1,2-a]pyridin-6-yl]-5-chlorophenoxy}ethyl)-N,1,5-trimethyl-1H-pyrazole-3-carboxamide)

### Methods

### Synthetic Procedures

### General Remarks

Compounds 1, 2, 3 and 4 were prepared using the synthetic procedures described in WO2017/001812. For the avoidance of doubt, Compounds 1, 2, 3 and 4 of the present invention correspond to Examples 77, 49, 35 and 31 of WO2017/001812 respectively.

Compound 6 may be prepared using the synthetic procedure described in WO2010/026365. For the avoidance of doubt, Compound 6 of the present invention corresponds to the compound DDD85646 of WO2010/026365.

Compounds 5 and 7 were prepared as described hereinbelow.

### General Experimental Details

### LC-MS

Compounds requiring purification under basic conditions were purified on an LC-MS system equipped with a YMC Actus Triart C18 5 μm (20×250 mm) column or Gemini NX 5 μm C18 (100×30 mm) column, using a gradient elution of acetonitrile in water containing 20 mM Ammonium bicarbonate (10-45% over 30 min then 95% acetonitrile for 2 minutes).

### HPLC

The purity of Compound 5 and 7 (was determined by analytical HPLC using an Eclipse Extend 5 μm C18 (150×4.6 mm) or Shimadzu L Column 2 ODS 5 μm C18 (150×4.6 mm) column using gradient elution of acetonitrile in water containing 10 mM ammonium acetate over 12 min.

### NMR

¹H NMR and ¹³C spectra were recorded on 400 MHz and 101 MHz respectively instruments at room temperature unless specified otherwise were referenced to residual solvent signals. Data are presented as follows: chemical shift in ppm, integration, multiplicity (br=broad, app=apparent, s=singlet, d=doublet, t=triplet, q=quartet, p=pentet, m=multiplet) and coupling constants in Hz.

### Synthetic Procedures

### Boc Deprotection (Method B)

The Boc protected amine was dissolved in dioxane and treated with a solution of HCl in dioxane (6M, 2 mL). The reaction mixture was stirred at room temperature overnight. All volatiles were removed under reduced pressure and the product triturated with ether redissolved in water and freeze dried.

### Preparation of Starting Materials

All of the starting materials for making the intermediate and example compound were obtained from commercial sources or using literature methods, except for methyl 4-(2-hydroxyethyl)-1,5-dimethyl-1H-pyrazole-3-carboxylate, which was made as follows:

### Preparation of methyl 4-(2-hydroxyethyl)-1,5-dimethyl-1H-pyrazole-3-carboxylate Starting Material

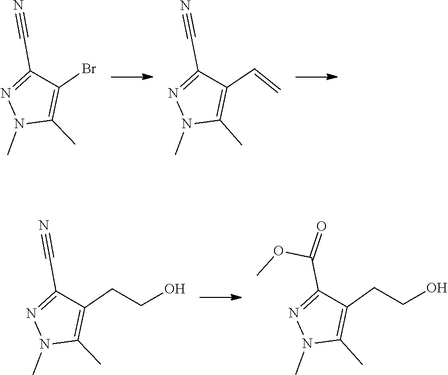

### Step 1

A solution of 4-bromo-1,5-dimethyl-1H-pyrazole-carbonitrile (8.0 g, 40 mmol) in dry DMF (40 mL) was treated with tributylvinylstannane (23.4 mL, 80 mmol). The mixture was purged with argon for 15 minutes before addition of tetrakis(triphenylphosphine) palladium(O) (2.3 g, 2 mmol). The reaction was heated to 110° C. overnight, diluted with ethyl acetate and washed with potassium fluoride solution, water and brine, dried over Na₂SO₄, concentrated under reduced pressure. The crude product was purified by flash column chromatography by elution with ethylacetate/hexane (20:80) to provide 4-ethenyl-1,5-dimethyl-1H-pyrazole-3-carbonitrile (4.0 g, 68%). ¹H NMR (400 MHz, CDCl₃) 6.45 (dd, 1H), 5.80 (dd, 1H), 5.34 (dd, 1H), 3.82 (s, 3H), 2.29 (s, 3H).

### Step 2

A solution of 4-ethenyl-1,5-dimethyl-1H-pyrazole-3-carbonitrile (1.2 g, 8.2 mmol) in dioxane (5 mL) was treated with a solution of 9-BBN (0.5M in THF, 32 mL, 16 mmol) under a nitrogen atmosphere. The reaction was heated to 100° C. overnight. The mixture was re-cooled to 0° C., and was treated with ethanol (4.8 mL), NaOH solution (6M, 2.4 mL), H₂O₂ (50% solution, 3.6 mL). The reaction mixture was heated at RT for 2 hr diluted with DCM/methanol (95:5), dried over sodium sulphate and concentrated under reduced pressure. The crude product purified by flash column chromatography by elution with DCM/methanol (98:2) to provide the compound 4-(2-hydroxyethyl)-1,5-dimethyl-1H-pyrazole-3-carbonitrile (500 mg, 37%). ¹H NMR (400 MHz, CDCl₃) 3.81 (s, 3H), 3.78 (q, 2H), 2.74 (t, 2H), 2.55 (s, 3H), 1.86 (t, 1H).

### Step 3

A solution of 4-(2-hydroxyethyl)-1,5-dimethyl-1H-pyrazole-3-carbonitrile (1.0 g, 6.1 mmol) in methanol (12 mL) was treated with a solution of HCl in dioxane (4M, 12 mL). The reaction mixture was stirred at 80° C. for 5 hr and evaporated under reduced pressure. The crude product was basified with sat. NaHCO₃ solution and diluted with EtOAc, washed with water, brine, dried over sodium sulfate and evaporated under reduced pressure to give methyl 4-(2-hydroxyethyl)-1,5-dimethyl-1H-pyrazole-3-carboxylate (1.1 g, 92%). ¹H NMR (400 MHz, CDCl₃) 3.90 (s, 3H), 3.84 (s, 3H), 3.77 (q, 2H), 2.93 (t, 2H), 2.23 (s, 3H), 2.07 (t, 1H).

### Preparation of Intermediate 1

### Intermediate 1

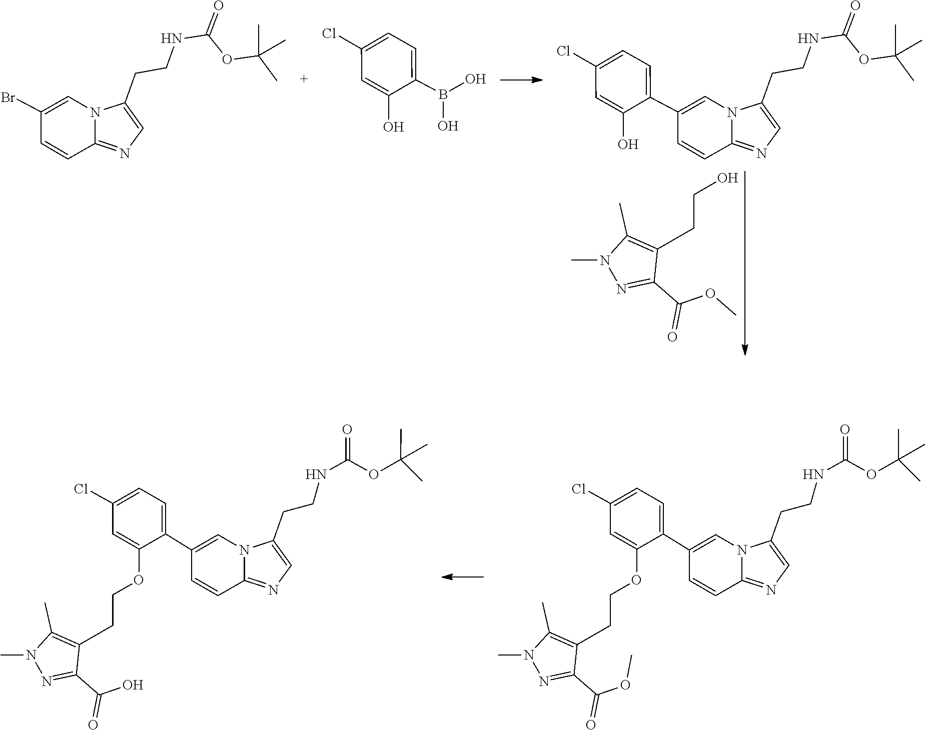

### 4-(2-{2-[3-(2-{(tert-butoxy)carbonyl]amino}ethyl)imidazo[1,2-a]pyridin-6-yl]-5chlorophenoxy}ethyl)-1,5-dimethyl-1H-pyrazole-3-carboxylic Acid

### Step 1

A solution of tert-butyl N-(2-{6-bromoimidazo[1,2-a]pyridin-3-yl}ethyl)carbamate (7.0 g, 20.5 mmol) was dissolved in dioxane/water (5:1, 175 mL) and treated with 4-chloro-2-hydroxybenzene boronic acid (8.0 g, 46.3 mmol) and tetrakis(triphenylphosphine) palladium(O) (937 mg, 2.0 mmol), followed by potassium phosphate (13 g, 61.7 mmol). The reaction mixture was purged with argon then heated to 100° C. for 3 hr, cooled to room temperature and filtered through a bed of Celite™ and washed with ethyl acetate. The ethyl acetate layer taken dried over Na₂SO₄, and evaporated under reduced pressure. The crude product was purified by column chromatography eluting with 3% MeOH in DCM to give tert-butyl N-{2-[6-(4-chloro-2-hydroxyphenyl)imidazo[1,2-a]pyridin-3-yl]ethyl}carbamate (7.98 g, 97%). ¹H NMR (400 MHz, DMSO-d₆) δ 8.49 (s, 1H), 7.43-7.62 (m, 4H), 6.97-7.01 (m, 3H), 5.76 (s, 1H), 3.27 (t, 2H), 3.05 (t, 2H), 1.34 (s, 9H).

### Step 2

A solution of methyl tert-butyl N-{2-[6-(4-chloro-2-hydroxyphenyl)imidazo[1,2-a]pyridin-3-yl]ethyl}carbamate (5.0 g, 12.9 mmol) in toluene (50 mL) was reacted with methyl 4-(2-hydroxyethyl)-1,5-dimethyl-1H-pyrazole-3-carboxylate (3.07 g, 15.5 mmol) and cyanomethylene tributylphosphorane (6.77 mL, 25.8 mmol) at 100° C. for 16 hr. The reaction mixture was then diluted with ethyl acetate, and washed with water and brine, dried over sodium sulphate and concentrated. This crude material was purified by column chromatography by elution with DCM: methanol (95:5) to give methyl 4-(2-{2-[3-(2-{[(tert-butoxy)carbonyl]amino}ethyl)imidazo[1,2-a]pyridin-6-yl]-5-chlorophenoxy}ethyl)-1,5-dimethyl-1H-pyrazole-3-carboxylate (3.8 g, 52%) as a brown gum. ¹H NMR (400 MHz, DMSO-d₆) δ 8.39 (s, 1H), 7.53 (d, 1H), 7.45 (s, 1H), 7.43 (d, 1H), 7.24-7.27 (m, 2H), 7.11 (dd, 1H), 6.97 (br, t, 1H), 5.75 (s, 1H), 4.14 (t, 2H), 3.71 (s, 3H), 3.68 (s, 3H), 2.99-3.03 (m, 4H), 1.91 (s, 3H), 1.30 (s, 9H).

### Step 3

A solution of methyl 4-(2-{2-[3-(2-{[(tert-butoxy)carbonyl]amino}ethyl)imidazo[1,2-a]pyridin-6-yl]-5-chlorophenoxy}ethyl)-1,5-dimethyl-1H-pyrazole-3-carboxylate (3.0 g, 5.3 mmol) in THF-water (4:1, 50 mL) was treated with methanol (0.1 mL) followed by lithium hydroxide hydrate (444 mg, 10.6 mmol). The resulting mixture was stirred at rt for 16 hr. The reaction mixture was cooled to 0° C. and was acidified with saturated citric acid solution and extracted with DCM. The final organic layer was dried over sodium sulphate and concentrated to afford desired product 4-(2-{2-[3-(2-{[(tert-butoxy)carbonyl]amino}ethyl)imidazo[1,2-a]pyridin-6-yl]-5-chlorophenoxy}ethyl)-1,5-dimethyl-1H-pyrazole-3-carboxylic acid (2.7 g, 92%) as a brown solid. ¹H NMR (400 MHz, DMSO-d₆) δ 8.42 (s, 1H), 7.56 (d, 1H), 7.44-7.55 (m, 2H), 7.32 (d, 1H), 7.27 (s, 1H), 7.11 (d, 1H), 6.98 (m, 1H), 5.76 (s, 1H), 4.13 (t, 2H), 3.68 (s, 3H), 3.32 (t, 2H), 3.01-3.04 (m, 4H), 1.93 (s, 3H), 1.29 (s, 9H).

### Preparation of Compound 5

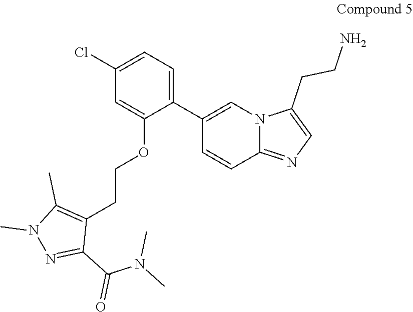 [SMILES: Cc1c(CCOc2cc(Cl)ccc2-c2ccc3ncc(CCN)n3c2)c(C(=O)N(C)C)nn1C]

### 4-(2-{2-[3-(2-aminoethyl)imidazo[1,2-a]pyridin-6-yl]-5-chlorophenoxy}ethyl)-N,N,1,5-tetramethyl-1H-pyrazole-3-carboxamide

### Step 1

A solution of 4-(2-{2-[3-({[2-(tert-butoxy)carbonyl]amino}ethyl)imidazo[1,2-a]pyridin-6-yl]-4-chlorophenoxy}ethyl)-1,5-dimethyl-1H-pyrazole-3-carboxylic acid (Intermediate 1, 1.8 g, 3.25 mmol) in THF (20 mL) was added carbonyldimidazole (790 mg, 4.9 mmol) and the reaction mixture was stirred at rt for 3 hr. The mixture was treated with triethylamine (1.4 mL, 9.7 mmol) followed by dimethylamine solution (2M in THF, 3.2 mL, 6.5 mmol). The reaction mixture was stirred at rt for 16 hrs, quenched by addition of saturated sodium bicarbonate solution, extracted with ethyl acetate. The organic extract was dried over sodium sulphate and concentrated. The crude product was purified by prep TLC (3% MeOH/DCM) to give tert-butylN-[2-(6-{2-[2-(3-(dimethyl)carbamoyl-1,5-dimethyl-1H-pyrazol-4-yl)ethoxy]-4-chlorophenyl}imidazo[1,2-a]pyridin-3-yl)ethyl]-carbamate as an off-white solid (1.0 g, 53%). ¹H NMR (400 MHz, DMSO-d₆) δ 8.38 (s, 1H), 7.51 (d, 1H), 7.42-7.45 (m, 2H), 7.23-7.26 (m, 2H), 7.11 (dd, 1H), 6.98 (m, 1H), 5.76 (s, 1H), 4.12 (t, 2H), 3.65 (s, 3H), 3.36 (t, 2H), 3.03 (s, 3H), 3.00 (t, 2H), 2.90 (s, 3H), 2.84 (t, 2H), 1.95 (s, 3H), 1.29 (d, 9H).

### Step 2

According to the general method for Boc deprotection (method A) tert-butyl N-[2-(6-{2-[2-(3-(dimethyl)carbamoyl-1,5-dimethyl-1H-pyrazol-4-yl)ethoxy]-4-chlorophenyl}imidazo[1,2-a]pyridin-3-yl)ethyl]-carbamate (1.40 g, 2.4 mmol) was treated with a solution of HCl in ether (2M, 70 mL). The solution stirred at room temperature for 3 hr and was evaporated under reduced pressure. The crude product dissolved in water and freeze dried to give the title compound as an off white solid (1.23 g, 92%) hplc rt 6.3 min LC-MS MH⁺ 481; ¹H NMR (400 MHz, DMSO-d₆) δ 14.9 (br.s, 1H), 9.01 (s, 1H), 8.36 (br. s, 3H), 8.17 (s, 1H), 8.02 (dd, 2H), 7.55 (d, 1H), 7.32 (d, 1H), 7.17 (d, 1H), 4.14 (t, 2H), 3.70 (s, 3H), 3.48 (t, 2H), 3.19 (t, 2H), 3.07 (s, 3H), 2.91 (s, 3H), 2.86 (t, 2H), 2.07 (s, 3H).

### Preparation of Compound 7

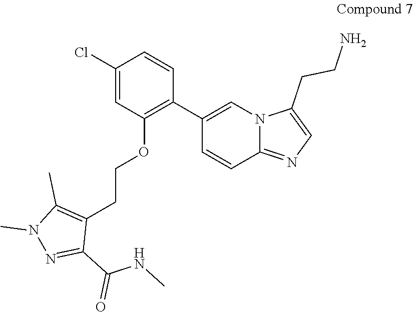 [SMILES: CNC(=O)c1nn(C)c(C)c1CCOc1cc(Cl)ccc1-c1ccc2ncc(CCN)n2c1]

### 4-(2-{2-[3-(2-aminoethyl)imidazo[1,2-a]pyridin-6-yl]-5-chlorophenoxy}ethyl)-N,1,5-trimethyl-1H-pyrazole-3-carboxamide

### Step 1

A solution of 4-(2-{2-[3-(2-{[(tert-butoxy)carbonyl]amino}ethyl)imidazo[1,2-a]pyridin-6-yl]-4-chlorophenoxy}ethyl)-1,5-dimethyl-1H-pyrazole-3-carboxylic acid (Intermediate 1, 64 mg, 0.19 mmol) in THF (2 mL) was treated with triethylamine (0.048 mL, 0.35 mmol), methylamine solution (2M in THF, 0.17 mL, 0.35 mmol), hydroxybenzotriazole (23.4 mg, 0.17 mmol) and EDCI (33.2 mg, 0.17 mmol). The reaction mixture was stirred at rt for 16 hrs, quenched with saturated NaHCO₃, extracted with ethyl acetate. The combined extracts were washed with water and brine, dried over sodium sulphate and concentrated. The crude product was purified by prep TLC (5% MeOH/DCM) to give tert-butyl N-{2-[6-(4-chloro-2-{2-[1,5-dimethyl-3-(methylcarbamoyl)-1H-pyrazol-4-yl]ethoxy}phenyl)imidazo[1,2-a]pyridin-3-yl]ethyl}carbamate as an off-white solid (30 mg, 46%). ¹H NMR (400 MHz, DMSO-d₆) δ 8.37 (s, 1H), 7.90 (d, 1H), 7.52 (d, 1H), 7.42-7.45 (m, 2H), 7.23-7.27 (m, 2H), 7.09 (d, 1H), 6.98 (t, 1H), 5.75 (s, 1H), 4.16 (t, 2H), 4.02 (q, 1H), 3.66 (s, 3H), 3.31 (t, 2H), 3.01 (m, 4H), 2.68 (s, 3H), 1.90 (s, 3H), 1.30 (d, 9H).

### Step 2

According to the general method for Boc deprotection (method B) tert-butyl N-{2-[6-(4-chloro-2-{2-[1,5-dimethyl-3-(methylcarbamoyl)-1H-pyrazol-4-yl]ethoxy}phenyl)imidazo[1,2-a]pyridin-3-yl]ethyl}carbamate (30 mg, 0.053 mmol) was dissolved in dioxane (2 mL), cooled to 0° C. and treated with a solution of HCl in ether (2M, 2 mL). The solution stirred at room temperature for 3 hr and was evaporated under reduced pressure. The crude product dissolved in water and freeze dried to give the title compound as a light brown solid (20 mg, 81%) hplc rt 3.9 min LC-MS MH⁺ 467; ¹H NMR (400 MHz, DMSO-d₆) δ 14.7 (br.s, 1H), 8.98 (s, 1H), 8.19 (br. s, 3H), 8.15 (s, 1H), 8.05 (d, 1H), 8.01 (d, 1H), 7.89 (q, 1H), 7.55 (dd, 1H), 7.37 (d, 1H), 7.19 (dd, 1H), 4.18 (t, 2H), 3.72 (s, 3H), 3.46 (t, 2H), 3.20 (q, 2H), 3.04 (t, 2H), 2.67 (d, 3H), 2.07 (s, 3H).

### Biological Data

### Materials

### Cell Lines

### P-493-6

The P-493-6 cell line is a non-transformed B cell line that can be genetically engineered to have either “low” levels of c-Myc, “medium” levels of c-Myc or “high” levels of c-Myc. The terms “low”, “medium” and “high” are descriptors commonly used in the art and will be readily understood to mean:
i) “low” levels of c-Myc—a level of MYC expression which represents non-proliferating normal cells (i.e. a level of MYC expression not sufficient to cause the cycling of the cells);
ii) “medium” levels of c-Myc—a level of MYC expression which represents normal proliferating cells; and
iii) ““high” levels of c-Myc—a level of MYC expression which represents enforced/deregulated expression of c-Myc, typically observed in cancerous cells.

The P-493-6 cell lines were sourced from Chi Van Dang, Ludwig Cancer Research. The modified P-493-6 cell lines may be established using methodology well known in the art, see for example Int. J. Cancer, 2000, 87(6), 787-793.

### SKNAS and Shep

The SKNAS and Shep cell lines are both human neuroblastoma cell lines. The SKNAS cell line used herein was sourced from Linda Valentijn, Academic Medical Center, Amsterdam. The Shep cell line used herein was sourced from Michael D. Hogarty, The Children's Hospital of Philadelphia. The SKNAS and Shep cell lines used herein may be prepared using methods known in the art, see, for example, Cancer Res., 2005, 65(8), 3136-3145 and Oncogene, 2008, 27(24), 3424-3434 respectively.

In the experimental protocols described herein the SKNAS and Shep cell lines were genetically engineered to model differences between high grade neuroblastoma (which are usually marked by MYCN amplifications and with particular poor clinical prognosis) and low grade neuroblastoma. Accordingly, the SKNAS and/or Shep cell lines “with N-MYC induction” described herein will be understood to be cell lines treated with tamoxifen and represent aggressive, high grade type cancer cells. Conversely, the SKNAS and/or Shep cell lines “without N-MYC induction” described herein will be understood to be cell lines not treated with tamoxifen and represent less aggressive forms of cancer cells (Huang et al., Cold Spring Harb. Perspect. Med., 2013, 3(10), 1-22).

### General Experimental

### Cell Culture:

Shep-ER-N-Myc, SKNAS-ER-N-Myc, and the P-493-6 cell line were cultured in DMEM high glucose (ThermoFisher Scientific, 61965059), supplemented with 10% FBS at 5% CO₂. The BL41 cell line was cultured in RPMI 1640 (ThermoFisher Scientific, 61870044), supplemented with 10% FBS at 5% CO₂. HeLa cells were cultured in DMEM low glucose (Sigma, D6046), supplemented with 10% FBS at 10% CO₂. The Shep-ER-N-Myc was sourced from Linda Valentijn, and firstly reported in Valentijn et. al., Cancer Res, 2005, 65(8), 3136-3145. The SKNAS-ER-N-Myc was sourced from Michael D. Hogarty, and firstly reported in Ushmorov et. al., Oncogene, 2008, 27, 3424-3434. The P-493-6 cell line was sourced from Chi Van Dang, and firstly reported in Pajic et. al., Int J Cancer, 2000, 87(6), 787-793. BL41 and HeLa cell lines were sourced from the Cell Service Standard Technology Platform at the Francis Crick Institute.

### Western Blot:

Shep (200,000/well) and SKNAS (500,000/well) were seeded on a 6 well plate on day 0. On day 1, media was exchanged and media containing 100 nM Tamoxifen (H6278 Sigma) was added to the cells. Ethanol was used as control. 24 and 48 hours later, cells were lysed in cold buffer (PBS, 1% Tryton, 0.1% SDS). Protein concentration was measured with BCA assay (ThermoFisher Scientific, 23250) according to the manufacturer instructions. 20 μg of proteins were loaded on 10% acrylamide gel and transferred onto Nitrocellulose membrane. Membrane was blocked in 1×TBS 0.1% Tween-20 containing 5% non-fat milk for 1 hour. N-Myc antibody (Cell Signalling, 9405) was prepared in 5% BSA 1×TBS 0.1% Tween-20™ and the membrane was left incubating at 4 degrees centigrade overnight with gentle shaking. The following day Glyceraldehyde 3-phosphate dehydrogenase (GAPDH, Cell Signalling, 2118) was added for 1 hour as loading control in 5% BSA 1×TBS 0.1% tween-20. Membranes were washed 3 times in TBS 0.1%. Membranes were developed using secondary anti-rabbit (HRP-DAKO) antibody using an IMAGEQUANT 600 RGB.

### CellTiter Blue:

### General

At the respective end points of the experiments 20 μL/well of the CellTiter Blue Assay (Promega, G8081) were added and the cells were incubated at 37° C. for 3 hours. The fluorescence was measured at 570 nM using an EnVision™ plate reader. As a positive control, the highest DMSO concentration was added to the cells (usually 0.4% DMSO); as a negative control, the cells were treated with 10 μg/mL Puromycin and 1 uM Staurosporine (Merck, P7255 and S4400, respectively).

### P-493-6

The experiment was performed in a 96 well plate and in technical quadruplicate. On day 0, 2500 cells/well in 50 μL media were seeded in, for the MYC high condition: just media; for the MYC medium condition: 1 uM β-Estradiol (Merck, E8875) and 0.1 μg/mL Doxycycline (Merck, D9891); for the MYC low condition: 0.1 μg/mL Doxycycline. On day 1, 50 μL of media for the respective MYC condition and 2×NMT inhibitor, in the respective concentration, were added to the respective wells. On day 3 (for 48 hours incubation) or day 4 (for 72 hours) of incubation with the NMT inhibitor, the experiment was stopped according to the general experimental procedures for the CellTiter Blue experiments.

### Shep:

The experiment was performed in a 96 well plate and in technical quadruplicate. On day 0, 1,000 cells/well were seeded in 50 μL media. On day 1, 50 μL media containing 200 nM Tamoxifen (Merck, H7904), to induce N-Myc, or ethanol as control were added to the medium for 24 hours. On day 2, 100 μL of medium for the respective N-Myc condition and 2×NMT inhibitor, in the respective concentration, were added to the wells. On day 4 (for 72 hours incubation), the experiment was stopped according to the general experimental procedures for the CellTiter Blue experiments.

### SKNAS:

The experiment was performed in a 48 well plate and in technical triplicate. On day 0, 5,500 cells/well were seeded in 200 μL media. On day 1, the media was removed and 200 μL of media with Tamoxifen, to induce N-Myc, or ethanol as control were added. On day 2, the media was removed and 200 μL of medium for the respective N-Myc condition and 1×NMT inhibitor, in the respective concentration, were added. On day 6 (for 96 hours of incubation), the experiment was stopped according to the general experimental procedures for the CellTiter Blue experiments.

### Flow Cytometry

### General

Single-cell suspensions were stained with the following monoclonal antibody: α-Active Caspase3 (BD Biosciences, C92-605, 550821, PE) and c-Myc (Abeam, Y69, ab190560). To analyse DNA content, we stained with FxCycle Violet (ThermoFisher Scientific, F10347). To assess proliferation, we used the Click-iT EdU Alexa Fluor 488 Flow Cytometry Assay Kit (ThermoFisher Scientific, C10420) according to manufacturer's instructions. Zombie NIR (Biolegend, 423105) was used to exclude dead cells. The cells were fixed in a 4% solution of PFA, permeabilised with Cytofix/Cytoperm (BD Bioscience, 554714) and washed in Perm/Wash Buffer (BD Bioscience, 554723). Samples were acquired on a MACSQuant VYB (Miltenyi Biotec) and analysed using the FlowJo software (v10.4, Tree star).

### P-493-6

The experiment was performed in a 96 U-well plate and in technical triplicate to validate the system and in technical duplicate to study the effect of NMT inhibitors in the different MYC states.

For the validation of the system, on day 0, 5,000 cells/well in 100 μL media were seeded in, for the MYC high condition: just media; for the MYC medium condition: 1 uM p-Estradiol (Merck, E8875) and 0.1 μg/mL Doxycycline (Merck, D9891); for the MYC low condition: 0.1 μg/mL Doxycycline. On day 3, the cells were treated according to the general procedures for flow cytometry.

To study the effect of the NMT inhibitors, on day 0, 2,500 cells/well in 50 μL were seeded in the respective media conditions for the different MYC conditions. On day 1 50 μL of media for the respective MYC condition and 2×NMT inhibitor, in the respective concentration, were added to the respective wells. After 6 hours, 24 hours, 48 hours and 72 hours the cells were treated according to the general procedures for flow cytometry. After 24 hours, to the plates for the 48 hours and 72 hours-time points 100 μL of fresh media, for the respective MYC condition and 1×NMT inhibitor for the respective concentration were added to the wells, to a total of 200 μL of media.

### Shep and SKNAS

The experiment was performed in a 24 well plate and in technical duplicate. On day 0, 7,800 cells/well for the Shep and 15,000 cells/well for the SKNAS were seeded in 500 μL media. On day 1, the media was removed and 600 μL of media with 100 nM of Tamoxifen, to induce N-Myc, or ethanol as control. On day 2, the respective concentrations of NMT inhibitor were added. After 6 hours, 24 hours, 48 hours and 72 hours the cells were treated according to the general procedures; however, all the media was collected to not loose apoptotic cells, and Trypsin (ThermoFisher Scientific, 25200056) was used to harvest the cells. The combined cells and media were collected in a tube and spun at 2000 g x 3 minutes, and transferred to a 96 well plate for further staining according to the general procedures for flow cytometry.

### RNA Sequencing

Samples were purified with the miRNeasy Mini Kit (Qiagen, 217004) according to manufacturer's instructions. The RNA sequencing was performed at the Advanced Sequencing Unit, The Francis Crick Institute. Samples were prepared with the Nugen cDNA kit and sequenced using Illumina HiSeq system. Gene expression raw data was analysed by the Bioinformatics and Biostatistics Unit, The Francis Crick Institute. The analysis was performed on biological replicates for each condition on biological replicates generating approximately 30 million 100 bp paired-end reads. The RSEM package (v1.3.0) (Li and Dewy, BMC Bioinformatics, 2011) and STAR (v2.5.2a) (Dobin et. al., Bioinformatics, 2013) were used to align reads to the human hg38 transcriptome, taken from Ensembl (v. GRChg38) available at UCSC (http://hqdownload.soe.ucsc.edu/downloads.html). For RSEM, all parameters were run as default using the “-forward-prob 0” option for strand specific protocol. Differential expression analysis was carried out with DESeq2 (v1.12.4) (Love, et. al., Genome Biol, 2014) within R (v3.3.1).

### BL41

The experiment was performed in T-25 flasks in biological quadruplicate. On day 0, 500,000 cells/mL were seeded in 10 mL of media. On day 1,100 nM of Compound 2 or DMSO as control were added. On day 2 (24 hours of incubation), the RNA was purified with the miRNeasy kit, according to manufacturer's instructions.

### HeLa

The experiment was performed in 10 cm² dishes, in biological quadruplicate. On day 0, 1,000,000 cells/dish were plated in 10 mL of media. On day 1, 100 nM of Compound 2 or DMSO as control were added. On day 2 (24 hours of incubation), the RNA was purified with the miRNeasy kit, according to manufacturer's instructions.

### Gene Set Enrichment Analysis

Gene set enrichment analysis (GSEA) was performed using GSEA (v3.0) (Subramanian et. al., PNAS, 2005, 102(43), 15545-15550). Gene sets were obtained from the Broad Institute Signatures data base (http://software.broadinstitute.org/gsea/msigdb/index.jsp). For the in-house RNAseq data, GSEA was carried out with ranked gene lists, using Wald statistics. All parameters were kept as default, except for the enrichment statistic (classic) and the max size that was changed to 500.000 respectively. For the microarray data, the pre-processed data was obtained from the Sanger Institute (Lorio et al., Cell, 2016, 166(3), 740-754; https://www.cancerrxgene.org/downloads). All parameters were kept as default, except the max size that was changed to 500,000 respectively. For the RNAseq data and CRISPR data sets from the Broad Institute, the pre-processed data was obtained from the DepMap project (CCLE and GDSC Consortium, Nature, 2015, 528, 84-87 and Robin et. al., Nature Genetics, 2017, 49(12), 1779-1784; https://depmap.org/portal/download/; Public 18Q4 Versions). All parameters in the GSEA were kept to default, except the max size that was changed to 500.000 respectively.

### Pharmacogenomics Screens

The sensitivity data, measured as EC50, for cancer cell lines treated with Compound 4, Compound 3, and Compound 6 were provided by the Sanger Institute, using their methods described in Lorio et. al., Cell, 2016,166(3), 740-754. The sensitivity data, measured as EC50, on Compound 3 and Compound 4 is not yet published; the sensitivity data for Compound 6 is available on: https://www.cancerrxgene.org/translation/Drug/1266. For the microarray data, copy number data, recurrently altered chromosomal segments data, and genomic variants, used to compare the expression of different genes or the presence/absence of structural alterations in the Myc paralogs loci with sensitivity, the pre-processed data was obtained from the Sanger Institute (https://www.cancerrxgene.org/downloads).

### Results

### NMT Inhibition

The NMT inhibition activity data for Compounds 1, 2, 3 and 4 is described in WO2017/001812. In particular, the NMT inhibition data is provided in Table 1 of WO2017/001812.

The inhibition activity data for Compound 6 is described in WO2010/026365, more specifically in the Table on page 118 of WO2010/026365.

### Compounds 5 and 7

### Example (a); HsNMT1 IC₅₀

The IC₅₀ values for human NMT1 (HsNMT1) of Compounds 5 and 7 were measured using a sensitive fluorescence-based assay based on detection of CoA by 7-diethylamino-3-(4-maleimido-phenyl)-4-methylcoumarin, as described in Goncalves, V., et al., Analytical Biochemistry, 2012, 421, 342-344 and Goncalves, V., et al., J. Med. Chem, 2012, 55, 3578.

The HsNMT1 IC₅₀ value for Compound 5 was observed to be 1.5 nM.

The HsNMT1 IC₅₀ value for Compound 7 was observed to be 0.4 nM.

### Example (b) Metabolic Activity Assay (MTS Assay)

Compounds 5 and 7 were also tested for activity in an in vitro metabolic activity assay using the human cell line MRC5. Compounds having activity in inhibiting metabolic activity in the assay are expected to be useful as agents for preventing and/or treating cancer, by virtue of being inhibitors of human NMT1 and/or NMT2. The compounds with the highest activity in the assay are expected to be the most potent inhibitors of human NMT1 and/or NMT2.

### Cell Preparation

MRC5 cells (cell type: fibroblast cells) were grown in DMEM media (supplemented with 10% FBS) and were seeded in a 96-well plate, 24 h prior to treatment. Cell suspensions were prepared by adjusting the cell density to the appropriate concentration (as stated in the Table 1 below) and 50 μL of the cell suspension was transferred to wells B-G in columns 2-11 of a 96-well plate.

TABLE 1
Number of cells plated

| | MRC5 |
|---|---|
| Cell suspension | 38,000 |
| concentration (cells/mL) | |
| cells per well | 1,900 |

### Assay Procedure

100 μL of growth media (DMEM media) containing 0.2% DMSO was added to wells B-G in columns 2 and 11 as positive controls, and 100 μL of growth media containing Puromycin (3 μg/mL; final concentration in the plate 2 μg/mL) was added to wells B-G in column 3 as a negative control. Seven concentrations of NMT inhibitor stock solution were prepared for Compound 5 (same final percentage of DMSO, dilution factor=3 starting from 15 μM or 150 μM). 100 μL of inhibitor stock solution was added to wells B-G in columns 4-10 of a 96-well plate (final concentration of Compound 5 in the plate starting from 10 μM or 100 μM; total volume in each well was 150 μL). The plate was incubated at 37° C. with 5% CO₂ level.

After 72 h, 20 μL MTS reagent (Promega, prepared according to the supplier protocol) was added to each well of the 96-well plate. The plate was incubated at 37° C. for 2 h and the absorbance per well was measured at 490 nm with an EnVision plate reader. The average absorbance value of the negative control (Puromycin-treated cells) was subtracted from each value and the metabolic activity was calculated as a percentage relative to the positive control (DMSO-treated cells). EC50 values were calculated using GraphPad.

The EC₅₀ value for Compound 5 was observed to be 13 nM.

The EC₅₀ value for Compound 7 was observed to be 37 nM.

### Correlations Between Alterations in MYC and the Effectiveness of a NMT Inhibitor

### P-493-6 Cell Lines

### Validation of the P-493-6 Cell Lines

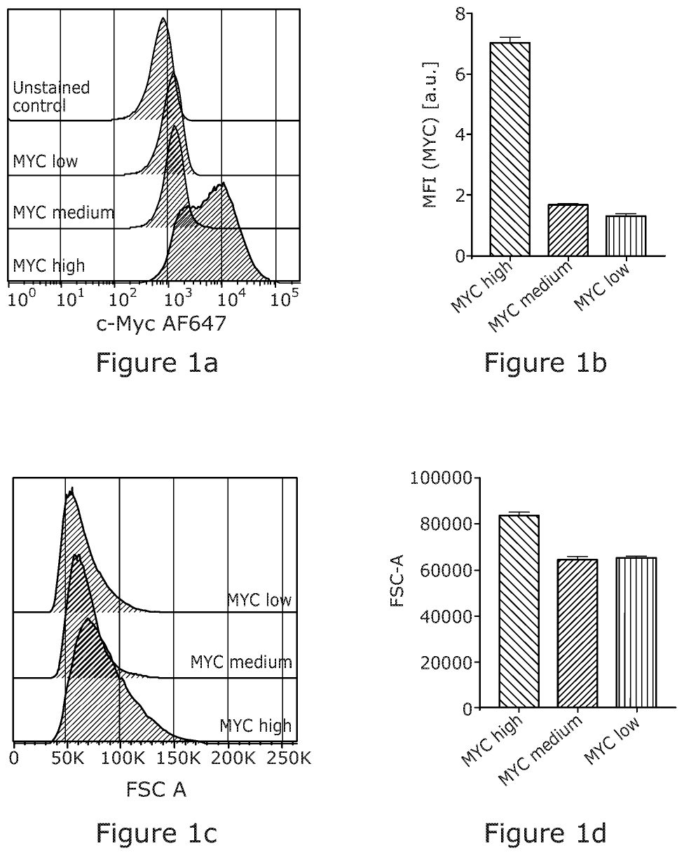

FIGS. 1, 2 and 3 show the flow cytometry analysis for the expression of c-MYC in the P-493-6 cell line. This data shows that the genetically engineered P-493-6 cell lines (immortalised B cell lines with an inducible system) allow for three different MYC levels: high, medium and low (as defined above). This makes the P-493-6 cell line a particularly good cell line to use for determining the correlation between the effectiveness of an NMT inhibitor and MYC expression.

High levels of MYC expression was found to cause increased cell size, which was measured and determined by increased forward scatter.

FIG. 1 shows: a) representative flow cytometry analysis for the expression of the c-Myc in the P-493-6 cell line from an unstained control (first row), low levels of c-Myc (second row), medium levels of c-Myc (third row), and high levels of c-Myc (fourth row); b) quantification of the median fluorescence intensities (MFI), normalised against the unstained control of the expression levels of c-Myc (N=3); c) representative flow cytometry analysis for the forward scatter area (FSC-A) as a surrogate for cell size in the P-493-6 cell line from low levels of c-Myc (first row), medium levels of c-Myc (second row), and high levels of c-Myc (third row); and d) quantification of the FSC-A geometric mean (N=3).

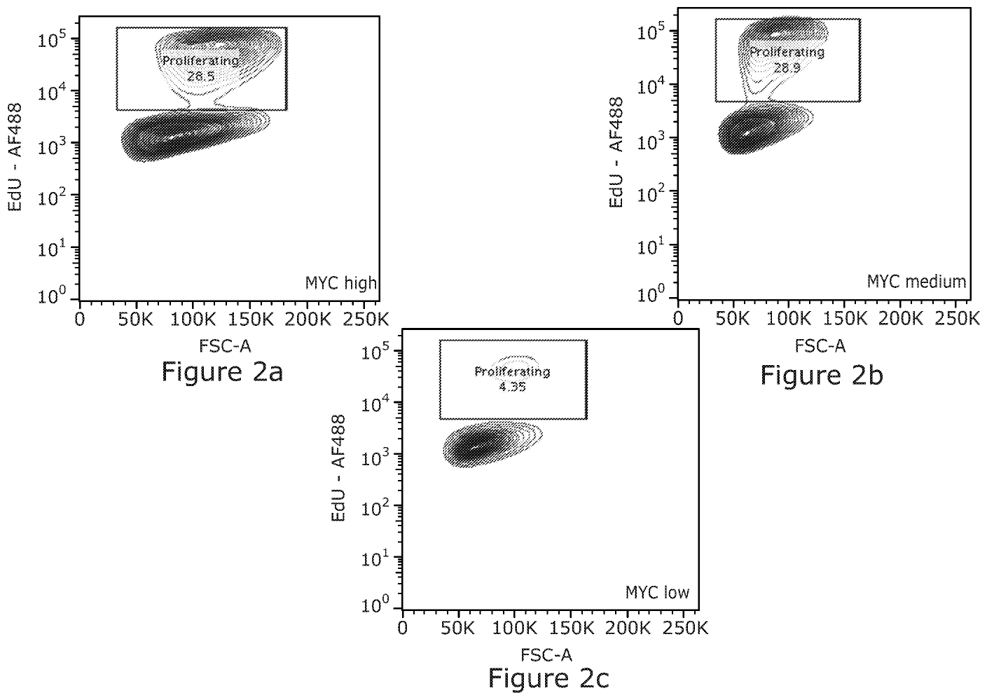

FIG. 2 shows: a) representative flow cytometry analysis of EdU incorporation, clicked to an AlexaFluor488fluorochrom, in the c-Myc high P-493-6 cell line; b) representative flow cytometry analysis of EdU incorporation, clicked to an AlexaFluor 488 fluorochrom, in the c-Myc medium P-493-6 cell line; and c) representative flow cytometry analysis of EdU incorporation, clicked to an AlexaFluor 488 fluorochrom, in the c-Myc low P-493-6 cell line.

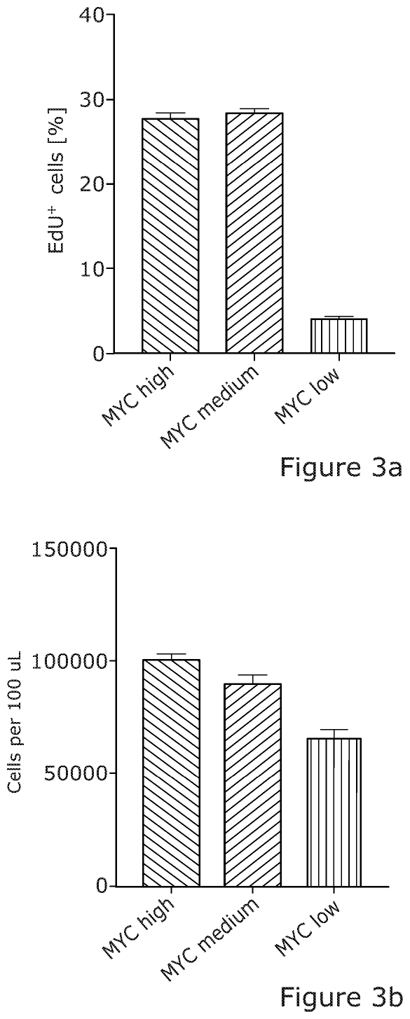

FIG. 3 shows: a) quantification of the relative proportion of cells that are EdU positive, hence engaging in DNA synthesis (N=3); b) quantification of the cell number in 100 uL of cell suspension, analysed via flow cytometry after 72 hours of induction (N=3).

While there is little difference between DNA synthesis between the P-493-6 cell lines with high, medium or low MYC levels (as measured by EdU incorporation), overall, the cell numbers were found to be higher in the MYC high state P-496-6 cell lines, indicating a potentially faster cycling.

### MYC Synthetic Lethality Data

FIG. 4 shows: a) metabolic viability of the P-493-6 cell line treated with Compound 2 at the shown concentrations for 48 hours, with different expression levels of c-Myc induced; b) metabolic viability of the P-493-6 cell line treated with Compound 2 at the shown concentrations for 72 hours, with different expression levels of c-Myc induced.

From FIG. 4 it can be seen that at both 48 hours and 72 hours there is a clear MYC-dependent increase in cell death in the P-493-6 cell lines. This data suggests that high levels of c-MYC are lethal in combination with an NMT inhibitor, such as Compound 2, and even administering the NMT inhibitor at a concentration as low as 10 nM resulted in measurable effects on cell viability at 72 hours in the Myc high cell lines.

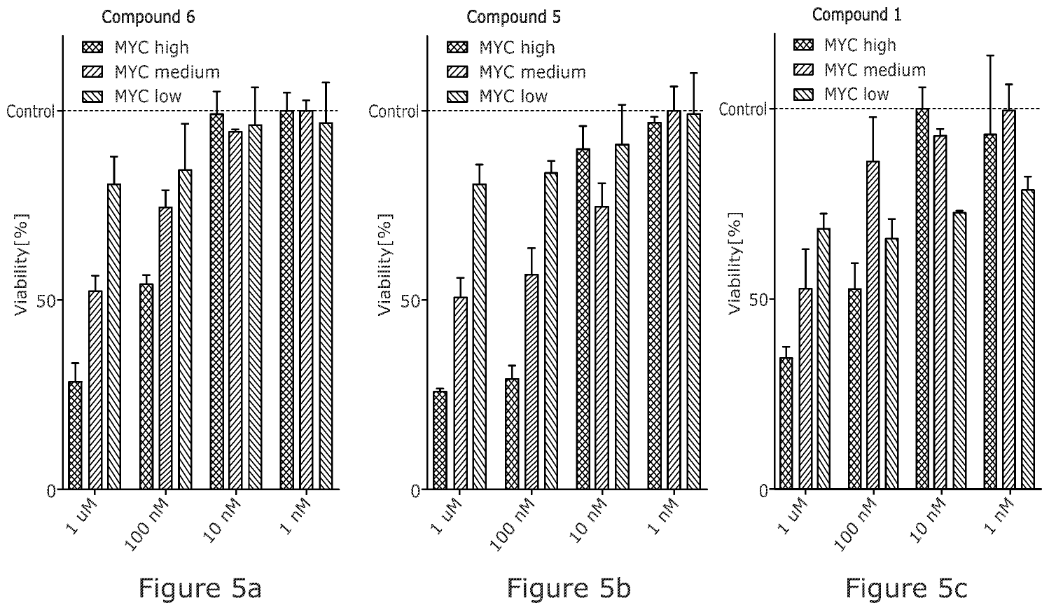

FIG. 5 shows: a) metabolic viability of the P-493-6 cell line treated with Compound 6 at the shown concentrations for 72 hours, with different expression levels of c-Myc induced; b) metabolic viability of the P-493-6 cell line treated with Compound 5 at the shown concentrations for 72 hours, with different expression levels of c-Myc induced; and c) metabolic viability of the P-493-6 cell line treated with Compound 1 at the shown concentrations for 72 hours, with different expression levels of c-Myc induced.

FIG. 5 shows that the MYC-dependent increase in cell death in the P-493-6 cell lines is observed for a range of structurally diverse NMT inhibitor compounds. With the highest levels of cell death being recorded in each case for P-493-6 cell lines with high MYC expression.

### Quantification of Cell Numbers

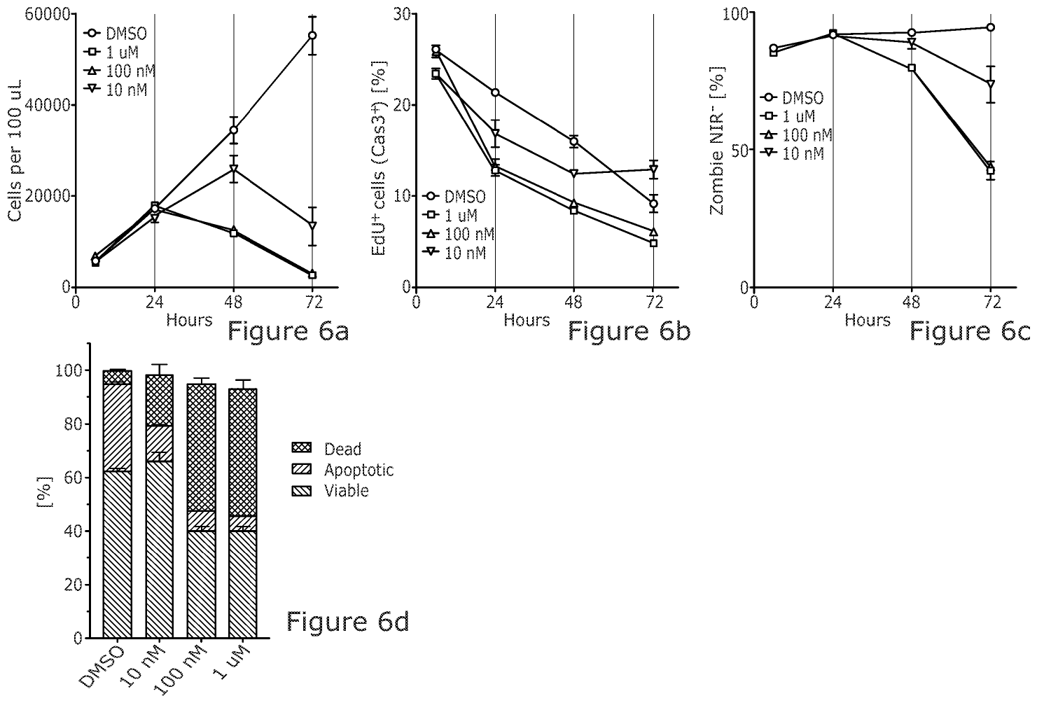

FIG. 6 shows: a) quantification of the cell numbers (excluding dead cells) of the P-493-6 cell line with c-MYC high, at the given time points (6, 24, 48 and 72 hours) with the shown concentrations (1 uM, 100 nM, 10 nM and control) of Compound 2 (N=2); b) quantification of the relative proportion of cells that are EdU positive, hence engaging in DNA synthesis, of the P-493-6 cell line with c-MYC high, at the given time points (6, 24, 48 and 72 hours) with the shown concentrations (1 uM, 100 nM, 10 nM and control) of Compound 2 (N=2); c) quantification of the relative proportion of cells that are Zombie NIR negative, hence have intact cell membranes, of the P-493-6 cell line with c-MYC high, at the given time points (6, 24, 48 and 72 hours) with the shown concentrations (1 uM, 100 nM, 10 nM and control) of Compound 2 (N=2); and d) quantification of the relative proportion of cells that are either dead (Zombie NIR positive), undergoing apoptosis (Caspase3 positive) or viable (Zombie NIR negative and Caspase 3 negative) of the P-493-6 cell line with c-Myc high (N=2).

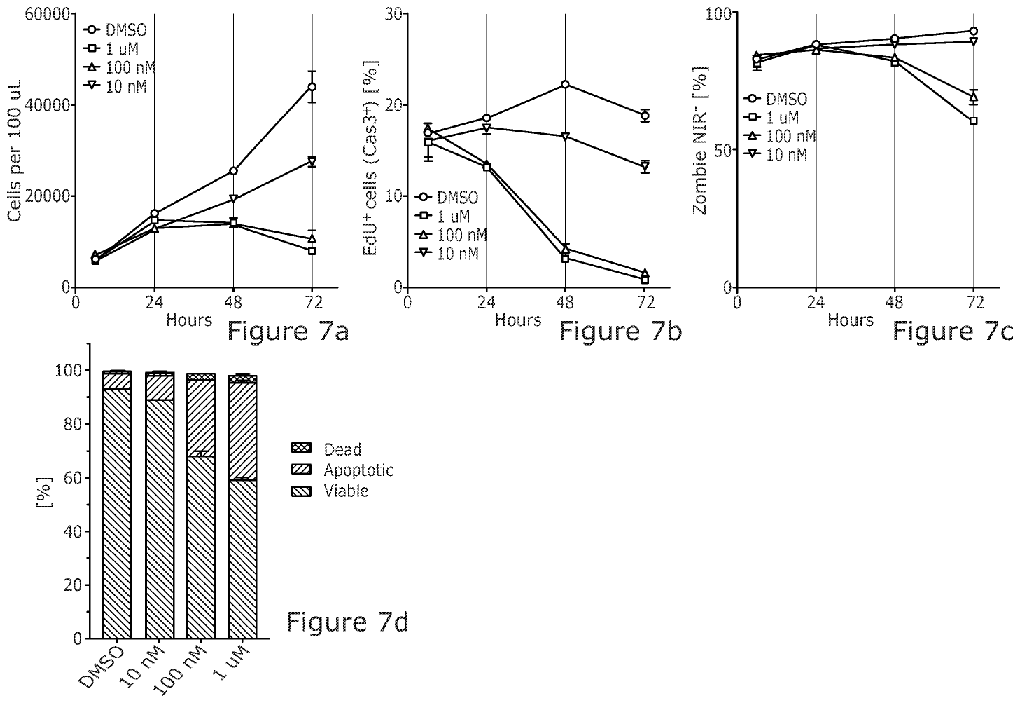

FIG. 7 shows: a) quantification of the cell numbers (excluding dead cells) of the P-493-6 cell line with c-MYC medium, at the given time points (6, 24, 48 and 72 hours) with the shown concentrations (1 uM, 100 nM, 10 nM and control) of Compound 2 (N=2); b) quantification of the relative proportion of cells that are EdU positive, hence engaging in DNA synthesis, of the P-493-6 cell line with c-MYC medium, at the given time points (6, 24, 48 and 72 hours) with the shown concentrations (1 uM, 100 nM, 10 nM and control) of Compound 2 (N=2); c) quantification of the relative proportion of cells that are Zombie NIR negative, hence have intact cell membranes, of the P-493-6 cell line with c-MYC medium, at the given time points (6, 24, 48 and 72 hours) with the shown concentrations (1 uM, 100 nM, 10 nM and control) of Compound 2 (N=2); and d) quantification of the relative proportion of cells that are either dead (Zombie NIR positive), undergoing apoptosis (Caspase3 positive) or viable (Zombie NIR negative and Caspase 3 negative) of the P-493-6 cell line with c-Myc medium.

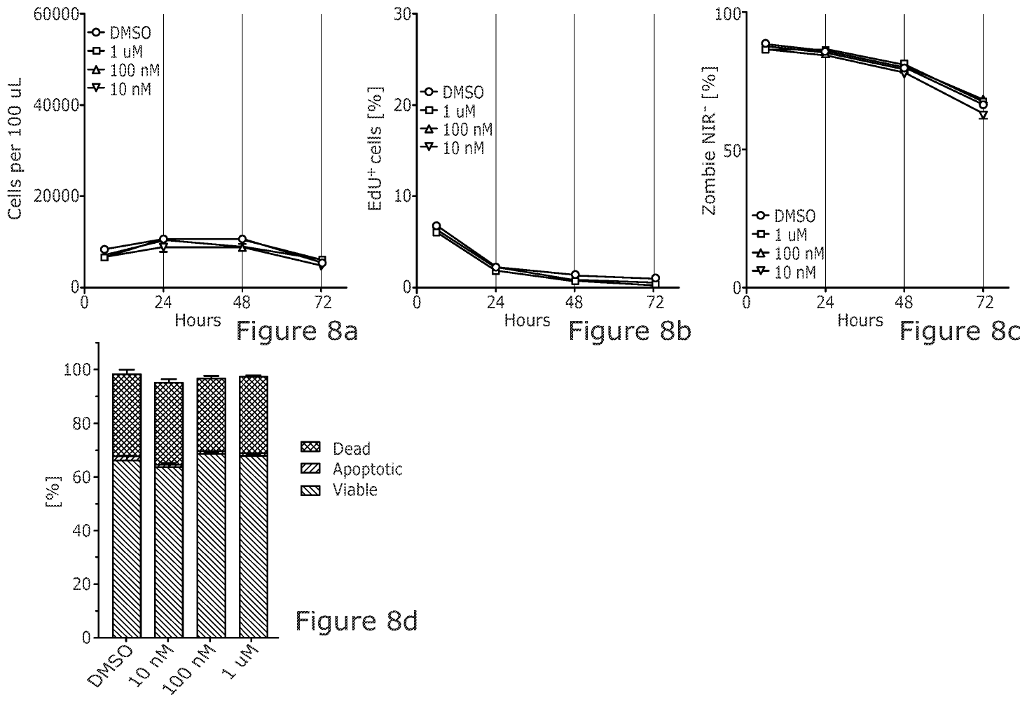

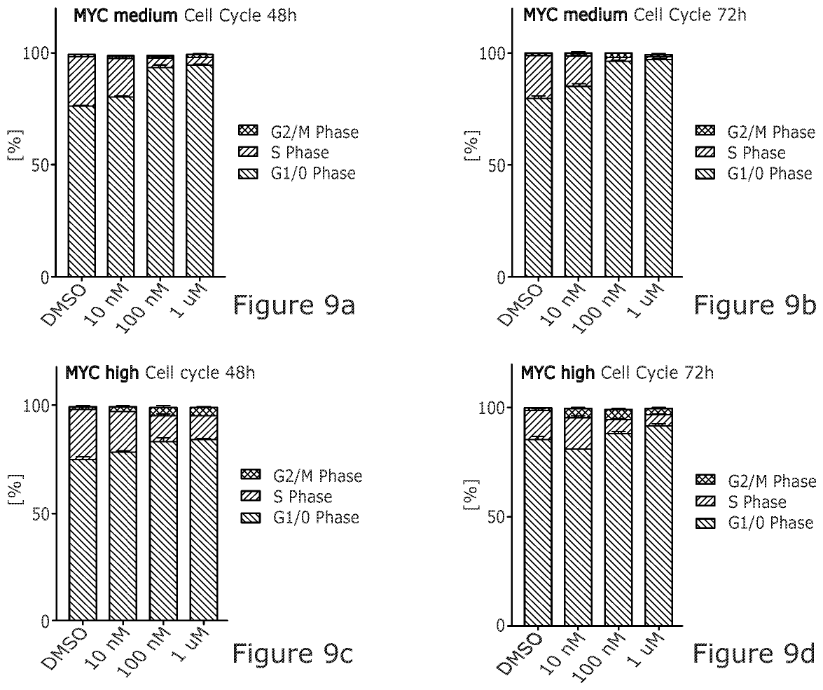

FIG. 8 shows: a) quantification of the cell numbers (excluding dead cells) of the P-493-6 cell line with c-MYC low, at the given time points (6, 24, 48 and 72 hours) with the shown concentrations (1 uM, 100 nM, 10 nM and control) of Compound 2 (N=2); b) quantification of the relative proportion of cells that are EdU positive, hence engaging in DNA synthesis, of the P-493-6 cell line with c-MYC low, at the given time points (6, 24, 48 and 72 hours) with the shown concentrations (1 uM, 100 nM, 10 nM and control) of Compound 2 (N=2); and c) quantification of the relative proportion of cells that are Zombie NIR negative, hence have intact cell membranes, of the P-493-6 cell line with c-MYC low, at the given time points (6, 24, 48 and 72 hours) with the shown concentrations (1 uM, 100 nM, 10 nM and control) of Compound 2 (N=2); and d) quantification of the relative proportion of cells that are either dead (Zombie NIR positive), undergoing apoptosis (Caspase3 positive) or viable (Zombie NIR negative and Caspase 3 negative) of the P-493-6 cell line with c-Myc low.

FIGS. 6, 7 and 8 show that high levels of c-MYC, in combination with an NMT inhibitor, cause rapid depletion of the cells. It can be seen from FIGS. 6, 7 and 8 that the MYC high cells are being depleted faster than the MYC medium and MYC low cells, with a rapid drop in overall cell viability. Interestingly, DNA synthesis in relative terms is less affected than in the MYC medium case. The MYC low cells are relatively unaffected by the administration of the NMT inhibitor compound.

### Shea ER-N-MYC and SKNAS ER-N-MYC Cell Lines

### Validation of the Shep ER-N-MYC and SKNAS ER-N-MYC Cell Lines

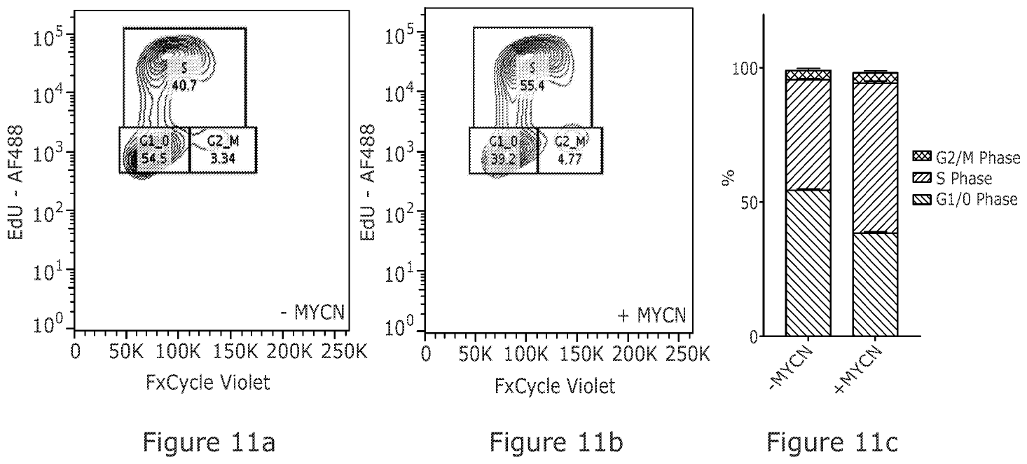

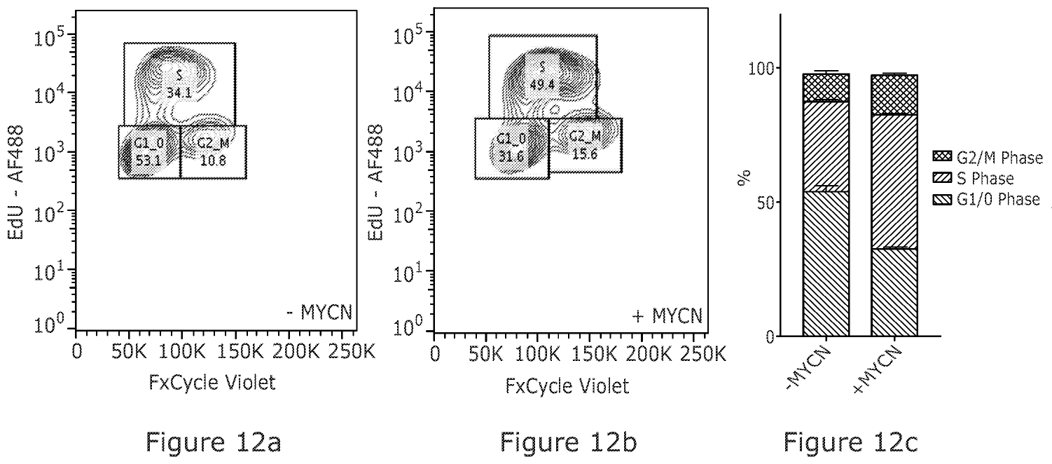

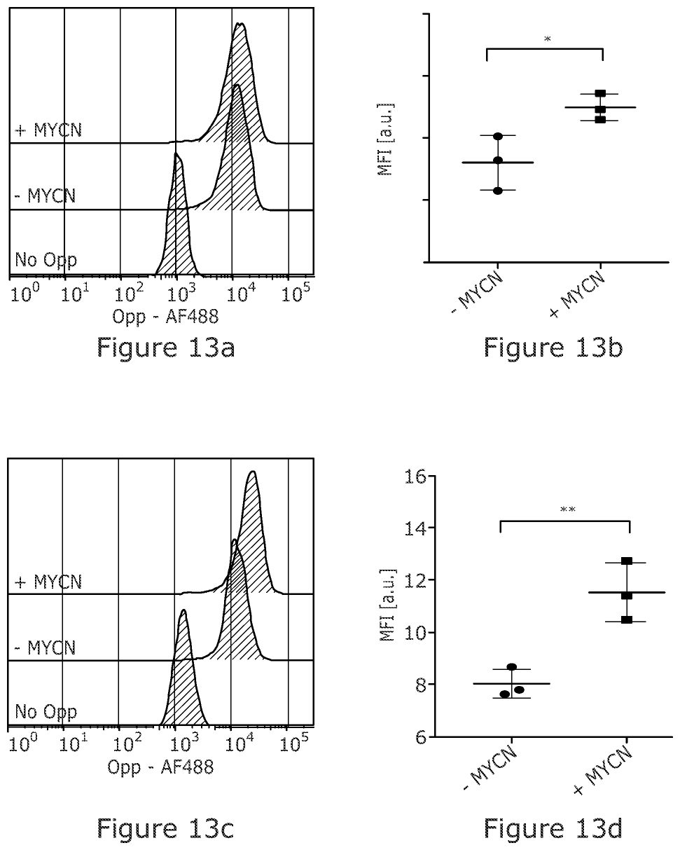

SKNAS and Shep are neuroblastoma cell lines, which usually have little to no MYCN expression. Upon treatment with tamoxifen, the cell line induces MYCN which increases cycling (seen in the top trace of FIGS. 11 and 12 measured by increase in S phase) and protein synthesis (seen in FIG. 13 and measured by OPP incorporation).

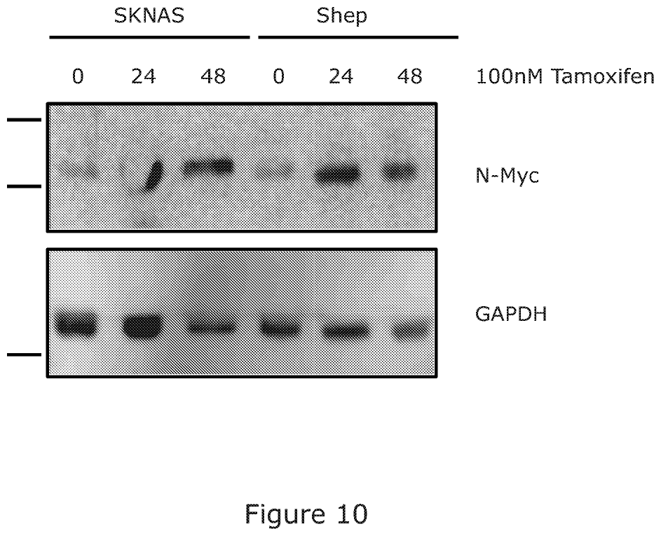

FIG. 10 shows representative western blot showing MYCN protein expression following time course treatment with 100 nM of Tamoxifen. GAPDH was used as loading control.

FIG. 11 shows: a) representative flow cytometric analysis of the cell cycle profile for the Shep ER-N-MYC cell line, without induction of MYCN. Cells in G1/0 phase have low amounts of FxCycle Violet stainings and are EdU negative. Cells in S phase are EdU positive. Cells in G2/M phase have high amounts of FxCycle Violet staining and are EdU negative; b) representative flow cytometric analysis of the cell cycle profile for the Shep ER-N-MYC cell line, with induction of MYCN. Cells in G1/0 phase have low amounts of FxCycle Violet stainings and are EdU negative. Cells in S pase are EdU positive. Cells in G2/M phase have high amounts of FxCycle Violet staining and are EdU negative; and c) quantification of the cell cycle profile of the Shep cell, with and without the induction of MYCN (N=3).

FIG. 12 shows: a) representative flow cytometric analysis of the cell cycle profile for the SKNAS ER-N-MYC cell line, without induction of MYCN. Cells in G1/0 phase have low amounts of FxCycle Violet stainings and are EdU negative. Cells in S pase are EdU positive. Cells in G2/M phase have high amounts of FxCycle Violet staining and are EdU negative; b) representative flow cytometric analysis of the cell cycle profile for the SKNAS ER-N-MYC cell line, with induction of MYCN. Cells in G1/0 phase have low amounts of FxCycle Violet stainings and are EdU negative. Cells in S pase are EdU positive. Cells in G2/M phase have high amounts of FxCycle Violet staining and are EdU negative; and c) quantification of the cell cycle profile of the SKNAS ER-N-MYC cell, with and without the induction of MYCN (N=3).

FIG. 13 shows: a) representative flow cytometric analysis of the protein synthesis, via incorporation of OPP, of the Shep ER-N-MYC cell line with or without induction of MYCN; first row: with induction of MYCN; second row: without induction of MYCN; last row: no OPP added; b) quantification of the median fluorescence of OPP, clicked to the AF488 fluorochrome, of the Shep ER-N-MYC cell line with and without induction of N-Myc; c) representative flow cytometric analysis of the protein synthesis, via incorporation of OPP, of the SKNAS ER-N-MYC cell line with or without induction of MYCN, first row: with induction of MYCN; second row: without induction of MYCN, last row: no OPP added; d.) quantification of the median fluorescence of OPP, clicked to the AF488 fluorochrome, of the SKNAS ER-N-MYC cell line with and without induction of MYCN.

### MYC Synthetic Lethality Data

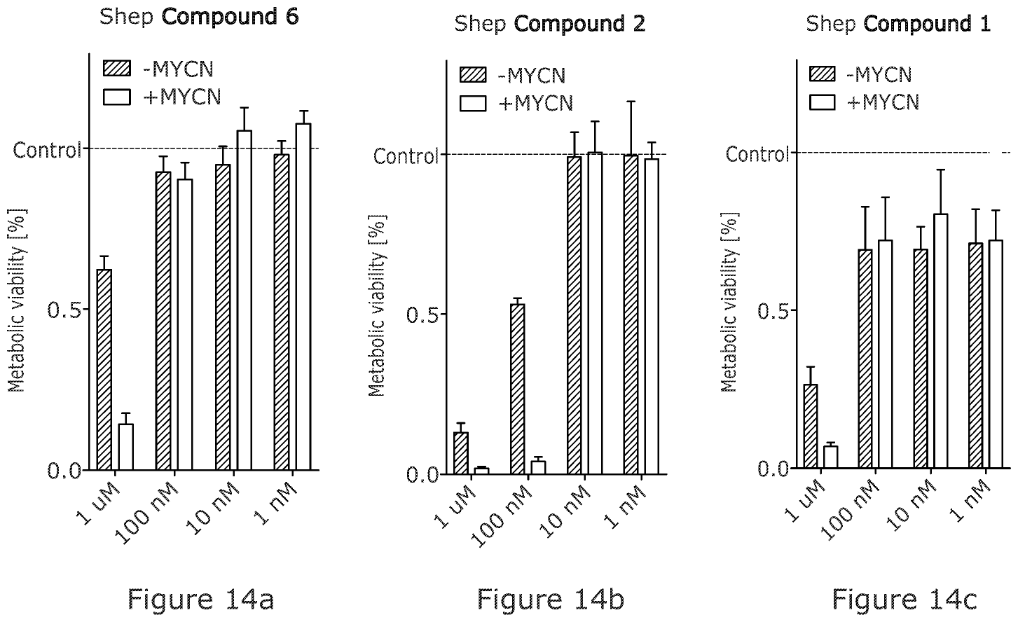

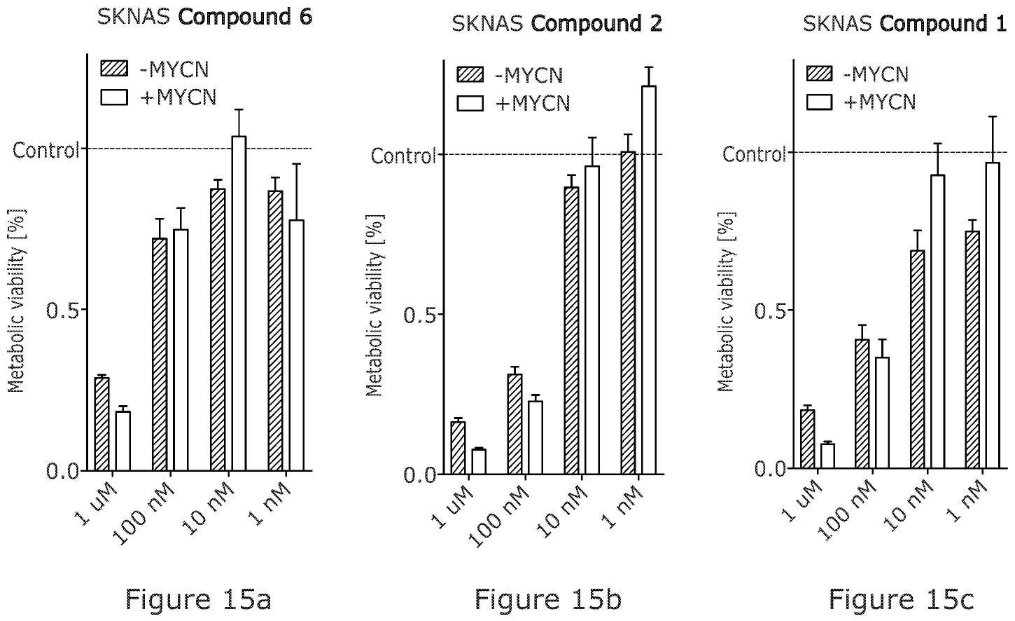

Similar to the case of c-MYC in the P-493-6 cell lines, it can be seen from FIGS. 14 and 15 that high levels of MYCN induce increased cell death more rapidly at lower concentrations upon treatment with Compound 2 than for low levels of MYCN. Similar results are observed when the Compounds 1 and 6 are administered to the Shep-ER-N-MYC and SKNAS-ER-N-MYC cell lines (see FIGS. 14 and 15).

FIG. 14 shows: a) metabolic viability of the Shep-ER-N-MYC cell line treated with Compound 6 at the shown concentrations for 72 hours, with (grey) or without (black) induction of MYCN; b) metabolic viability of the Shep-ER-N-MYC cell line treated with Compound 2 at the shown concentrations for 72 hours, with (grey) or without (black) induction of MYCN, c) metabolic viability of the SKNAS-ER-N-MYC cell line treated with Compound 1 at the shown concentrations for 72 hours, with (orange) or without (grey) induction of MYCN.

FIG. 15 shows the metabolic viability of the SKNAS-ER-N-MYC cell line treated with: a) Compound 6; b) Compound 2; and c) Compound 1 at the shown concentrations for 96 hours, with (grey) or without (black) induction of MYCN.

### Quantification of Cell Numbers

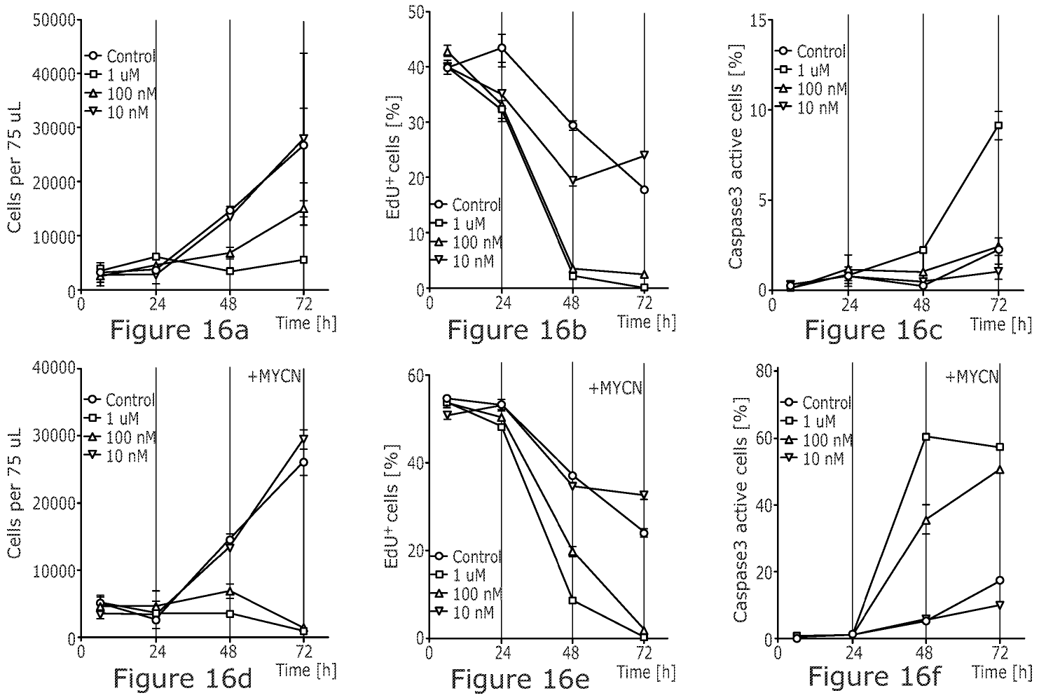

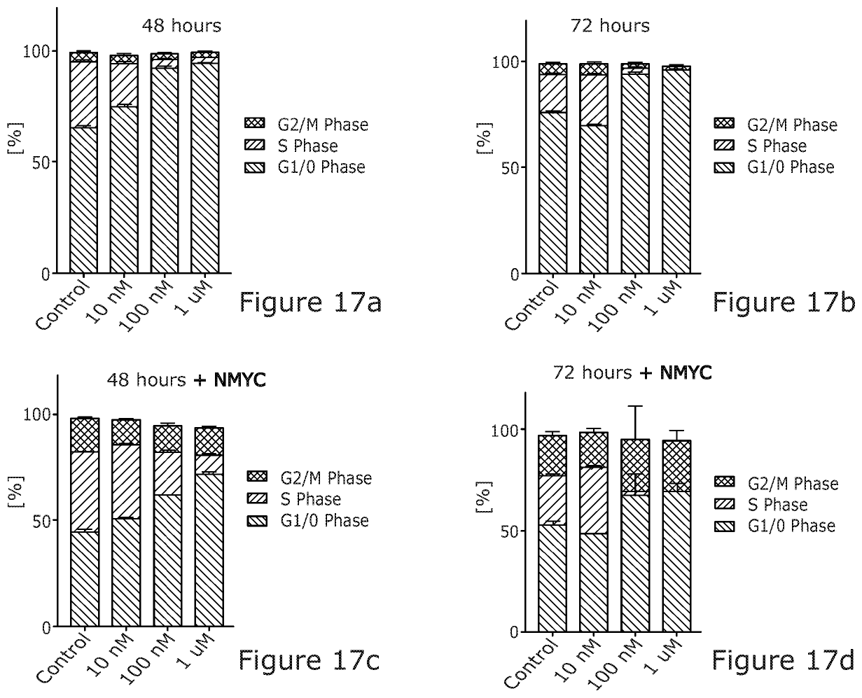

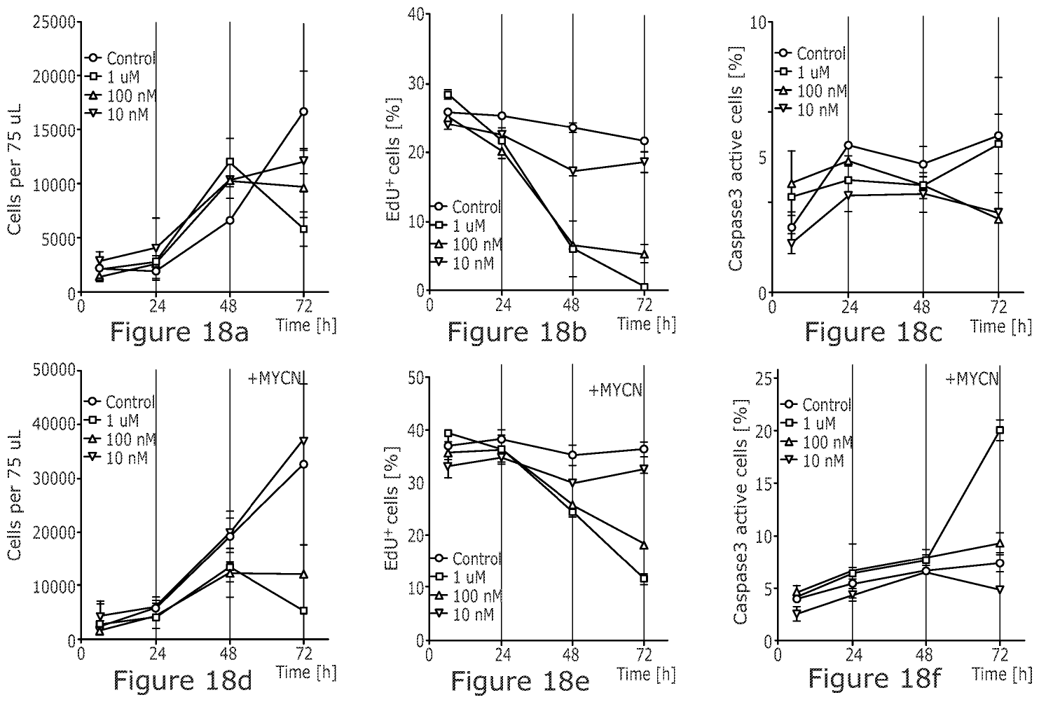

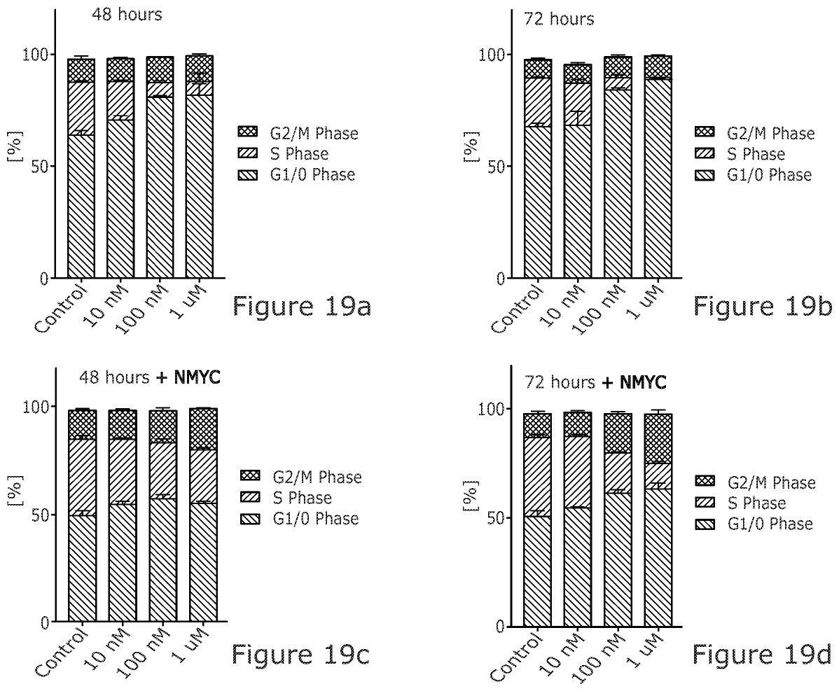

As can be seen from FIGS. 16 and 18, administration of a NMT inhibitor to a neuroblastoma cell with high levels of MYCN results in rapid depletion of the cells. For the Shep cell line in the high MYCN state, apoptosis induction occurs rapidly after 24 hours (up to 60%). In the case of no (or low) MYCN, this only occurs after 48 hours and to a much lesser degree. As similarly observed for c-MYC expression in the P-493-6 cell line, the Shep cells are still trying to cycle at 48 hours with high MYCN. (See FIG. 17) Similar results are observed for the SKNAS ER-N-MYC cell line (FIGS. 18 and 19).

FIG. 16 shows: a) quantification of the cell numbers (excluding dead cells) of the Shep-ER-N-MYC cell line without MYCN induction, at the given time points (6, 24, 48 and 72 hours) with the shown concentrations (1 uM, 100 nM, 10 nM and control) of Compound 2 (N=2); b) quantification of the relative proportion of cells that are EdU positive, hence engaging in DNA synthesis, of the Shep-ER-N-MYC cell line without MYCN induction, at the given time points (6, 24, 48 and 72 hours) with the shown concentrations (1 uM, 100 nM, 10 nM and control) of Compound 2 (N=2); c) quantification of the relative proportion of cells that caspase3 positive, hence are undergoing apoptosis, of the Shep-ER-N-MYC cell line without MYCN induction, at the given time points (6, 24, 48 and 72 hours) with the shown concentrations (1 uM, 100 nM, 10 nM and control) of Compound 2 (N=2). d) quantification of the cell numbers (excluding dead cells) of the Shep-ER-N-MYC cell line with MYCN induction, at the given time points (6, 24, 48 and 72 hours) with the shown concentrations (1 uM, 100 nM, 10 nM and control) of Compound 2 (N=2); e) quantification of the relative proportion of cells that are EdU positive, hence engaging in DNA synthesis, of the Shep-ER-N-MYC cell line with MYCN induction, at the given time points (6, 24, 48 and 72 hours) with the shown concentrations (1 uM, 100 nM, 10 nM and control) of Compound 2 (N=2); and f) quantification of the relative proportion of cells that caspase3 positive, hence are undergoing apoptosis, of the Shep-ER-N-MYC cell line with MYCN induction, at the given time points (6, 24, 48 and 72 hours) with the shown concentrations (1 uM, 100 nM, 10 nM and control) of Compound 2 (N=2).

FIG. 17 shows: a.) quantification of the cell cycle profile of the Shep-ER-N-Myc cell line without induction of MYCN, treated with the shown concentration of Compound 2 (1 uM, 100 nM, 10 nM and control) for 48 hours (N=2); b.) quantification of the cell cycle profile of the Shep-ER-N-Myc cell line without induction of MYCN, treated with the shown concentration of Compound 2 (1 uM, 100 nM, 10 nM and control) for 72 hours (N=2); c.) quantification of the cell cycle profile of the Shep-ER-N-Myc cell line with induction of MYCN, treated with the shown concentration of Compound 2 (1 uM, 100 nM, 10 nM and control) for 48 hours (N=2); and d.) quantification of the cell cycle profile of the Shep-ER-N-Myc cell line with induction of MYCN, treated with the shown concentration of Compound 2 (1 uM, 100 nM, 10 nM and control) for 72 hours (N=2).

FIG. 18 shows: a) quantification of the cell numbers (excluding dead cells) of the SKNAS-ER-N-MYC cell line without MYCN induction, at the given time points (6, 24, 48 and 72 hours) with the shown concentrations (1 uM, 100 nM, 10 nM and control) of Compound 2 (N=2); b) quantification of the relative proportion of cells that are EdU positive, hence engaging in DNA synthesis, of the SKNAS-ER-N-MYC cell line without MYCN induction, at the given time points (6, 24, 48 and 72 hours) with the shown concentrations (1 uM, 100 nM, nM and control) of Compound 2 (N=2); c) quantification of the relative proportion of cells that caspase3 positive, hence are undergoing apoptosis, of the SKNAS-ER-N-MYC cell line without MYCN induction, at the given time points (6, 24, 48 and 72 hours) with the shown concentrations (1 uM, 100 nM, 10 nM and control) of Compound 2 (N=2). d) quantification of the cell numbers (excluding dead cells) of the SKNAS-ER-N-MYC cell line with MYCN induction, at the given time points (6, 24, 48 and 72 hours) with the shown concentrations (1 uM, 100 nM, 10 nM and control) of Compound 2 (N=2); e) quantification of the relative proportion of cells that are EdU positive, hence engaging in DNA synthesis, of the SKNAS-ER-N-MYC cell line with MYCN induction, at the given time points (6, 24, 48 and 72 hours) with the shown concentrations (1 uM, 100 nM, 10 nM and control) of Compound 2 (N=2); and f) quantification of the relative proportion of cells that caspase3 positive, hence are undergoing apoptosis, of the Shep-ER-N-MYC cell line with MYCN induction, at the given time points (6, 24, 48 and 72 hours) with the shown concentrations (1 uM, 100 nM, 10 nM and control) of Compound 2 (N=2). FIG. 19 shows: a) quantification of the cell cycle profile of the Shep-ER-N-Myc cell line with induction of MYCN, treated with the shown concentration of Compound 2 (1 uM, 100 nM, 10 nM and control) for 48 hours (left) and 72 hours (right) (N=2); and b) relative change of the different cell cycle phases, shown as log 2-fold changes over the control for 100 nM Compound 2 at 48 hours and 72 hours for the Shep-ER-N-Myc cell line with induction of MYCN.

FIG. 19 shows: a.) quantification of the cell cycle profile of the SKNAS-ER-N-Myc cell line without induction of MYCN, treated with the shown concentration of Compound 2 (1 uM, 100 nM, 10 nM and control) for 48 hours (N=2); b.) quantification of the cell cycle profile of the SKNAS-ER-N-Myc cell line without induction of MYCN, treated with the shown concentration of Compound 2 (1 uM, 100 nM, 10 nM and control) for 72 hours (N=2); c.) quantification of the cell cycle profile of the SKNAS-ER-N-Myc cell line with induction of MYCN, treated with the shown concentration of Compound 2 (1 uM, 100 nM, 10 nM and control) for 48 hours (N=2); and d.) quantification of the cell cycle profile of the SKNAS-ER-N-Myc cell line with induction of MYCN, treated with the shown concentration of Compound 2 (1 uM, 100 nM, 10 nM and control) for 72 hours (N=2).

As can be seen from FIGS. 17 and 19, and as was similarly observed for c-MYC, MYCN forces the cells through the G1-S checkpoint increasing the lethality of the NMT inhibitor.

Furthermore, it was observed that the MYCN activated cells do not reduce their cycling even when presented with Compound 2 (see FIGS. 17 and 19), although there is a slight increase in G2/M accumulation. On the other hand, the cell line without activated MYCN was observed to stops cycling at 48 hours.

### Global Correlations

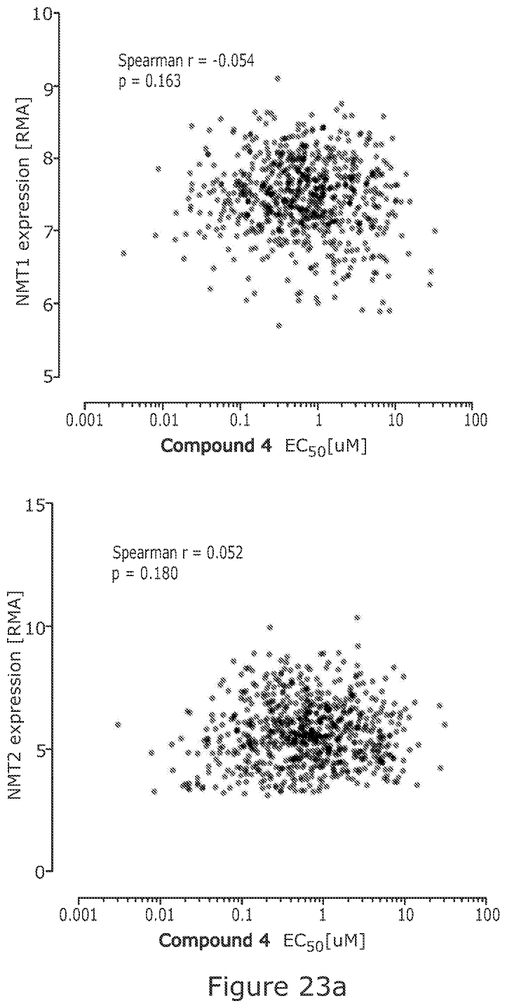

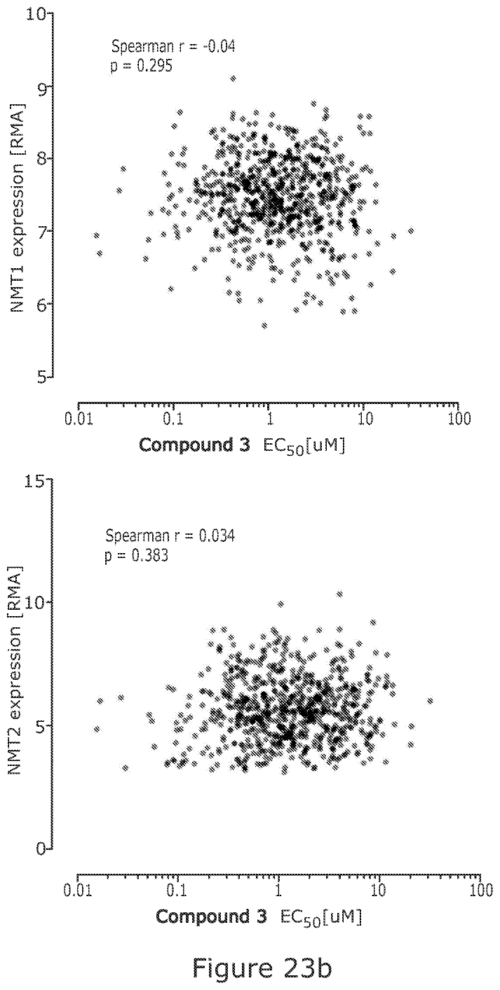

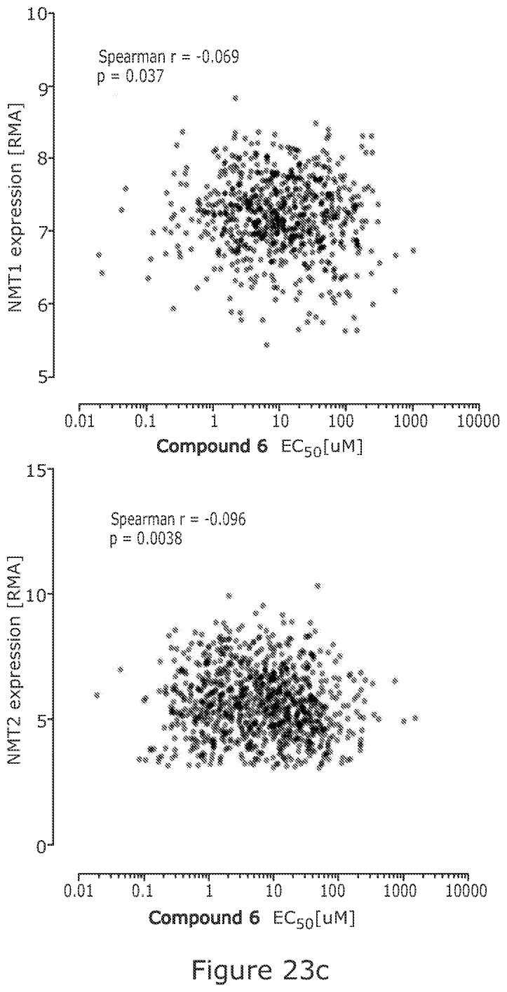

The GDSC data shown in FIGS. 20-22 indicates a correlation between c-MYC expression and sensitivity to an NMT inhibitor. A trend was observed indicating that increased c-MYC correlates well with higher sensitivity to the three different NMT inhibitors tested in the Sanger cancer line drug screen. Contrary to previous disclosures in the art (see, for example, WO2017/011907A1), FIG. 23 shows that there is no correlation between the expression of hsNMT1 or hsNMT2 with the sensitivity to an NMT inhibitor.

FIG. 20 shows: a) global comparison of the correlation between sensitivity to the NMT inhibitor Compound 6, measured as EC50, and normalised c-MYC expression (the expression data was obtained from Lorio et. al., Cell, 2016, 166(3), 740-754 and a nonparametric Spearman correlation coefficient was used); b) comparison between the top quartile and bottom quartile of cells with the highest (dashed/grey) and lowest expression (black) and their respective responsiveness to the NMT inhibitor Compound 6 (a nonparametric Mann Whitney test was used).

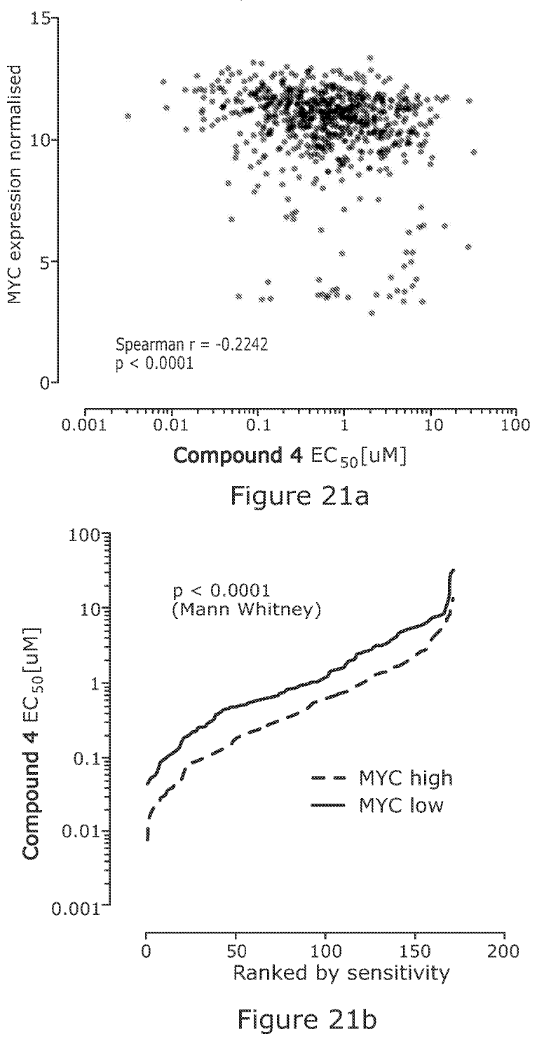

FIG. 21 shows: a) global comparison of the correlation between sensitivity to the NMT inhibitor Compound 4, measured as EC50, and normalised c-MYC expression (the expression data was obtained from Lorio et. al., Cell, 2016, 166(3), 740-754 and a nonparametric Spearman correlation coefficient was used); and b) comparison between the top quartile and bottom quartile of cells with the highest (dashed/grey) and lowest expression (black) and their respective responsiveness to the NMT inhibitor Compound 4 (a nonparametric Mann Whitney test was used).

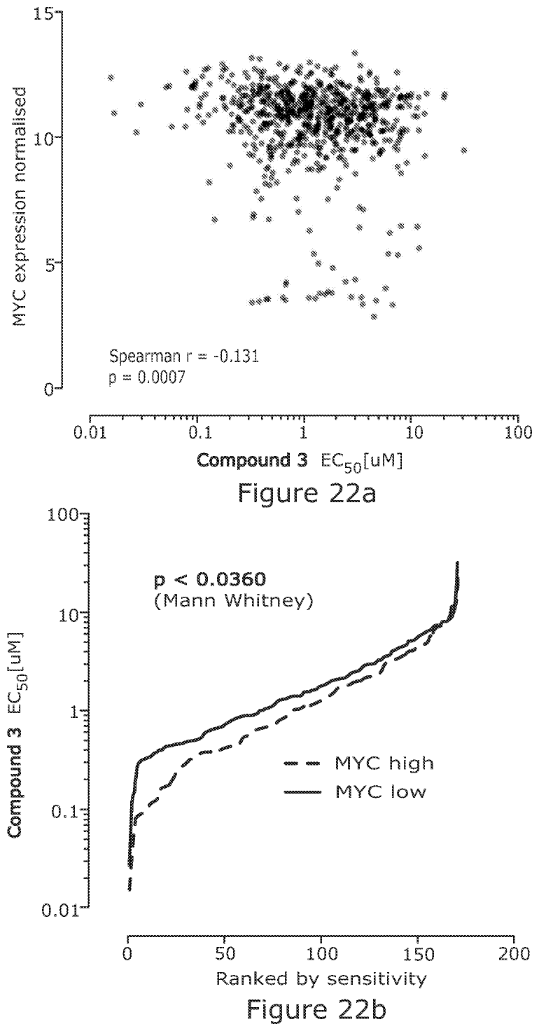

FIG. 22 shows: a) global comparison of the correlation between sensitivity to the NMT inhibitor Compound 3, measured as EC50, and normalised c-MYC expression (the expression data was obtained from Lorio et. al., Cell, 2016, 166(3), 740-754 and a nonparametric Spearman correlation coefficient was used); b) comparison between the top quartile and bottom quartile of cells with the highest (dashed/grey) and lowest expression (black) and their respective responsiveness to the NMT inhibitor Compound 3 (a nonparametric Mann Whitney test was used).

FIG. 23 shows: a.) top: global comparison of the correlation between sensitivity to the NMT inhibitor Compound 4, measured as EC50, and normalised NMT1 expression; bottom: global comparison of the correlation between sensitivity to the NMT inhibitor Compound 4, measured as EC50, and normalised NMT2 expression (the expression data was obtained from Lorio et. al., Cell, 2016, 166(3), 740-754 and a nonparametric Spearman correlation coefficient was used); b.) top: global comparison of the correlation between sensitivity to the NMT inhibitor Compound 3, measured as EC50, and normalised NMT1 expression; bottom: global comparison of the correlation between sensitivity to the NMT inhibitor Compound 3, measured as EC50, and normalised NMT2 expression (the expression data was obtained from Lorio et, al., Cell, 2016, 166(3), 740-754 and a nonparametric Spearman correlation coefficient was used); and c.) top: global comparison of the correlation between sensitivity to the NMT inhibitor Compound 6, measured as EC50, and normalised NMT1 expression; bottom: global comparison of the correlation between sensitivity to the NMT inhibitor Compound 6, measured as EC50, and normalised NMT2 expression (the expression data was obtained from Lorio et. al., Cell, 2016, 166(3), 740-754 and a nonparametric Spearman correlation coefficient was used).

mRNAseq Data

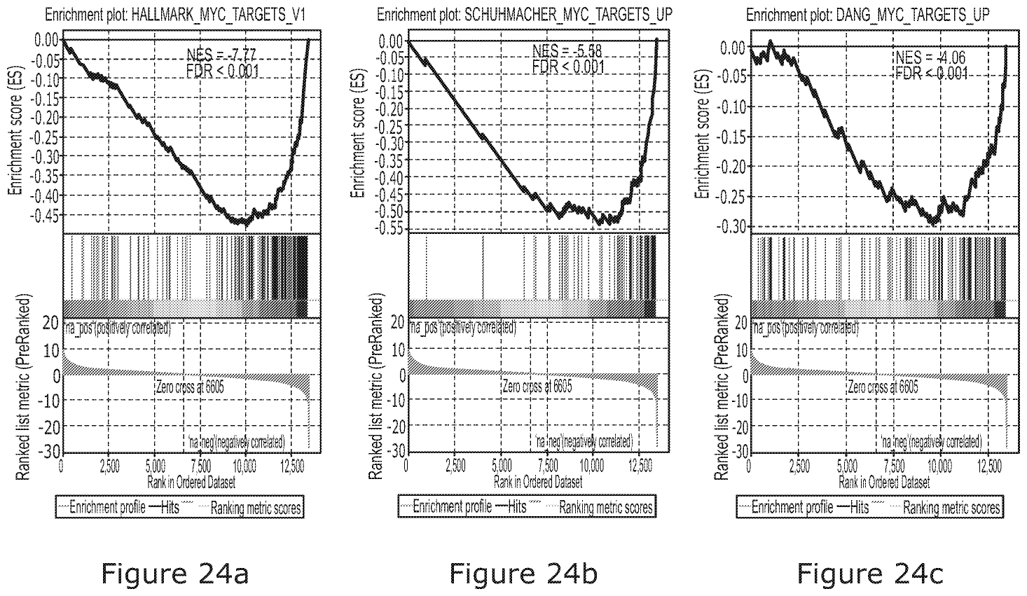

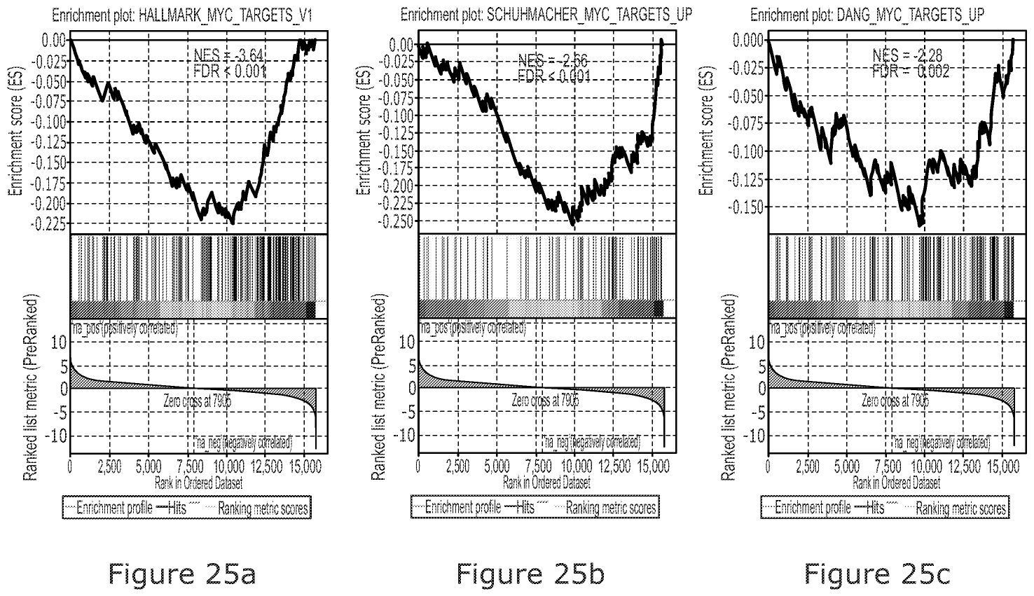

Looking at the mRNAseq data sets for BL41 and HeLa (FIGS. 24 and 25), in particular the case of BL41, it is observed that the c-MYC gene sets collapse to a much greater extent than in HeLa. This potentially indicates that the c-MYC driven program is particularly disturbed in BL41, a Burkitt's cell line with c-MYC translocations. Thus, the mRNAseq in BL41 indicates an increased breakdown of the MYC transcriptional program upon administration of a NMT inhibitor compound in a c-MYC addicted cancer.

FIG. 24 shows the effect of 100 nM Compound 2 for 24 hours on different MYC gene sets (obtained from the Broad Institute: http://software.broadinstitute.org/gsea/index.jsp), as measured via mRNAseq in BL41, a Burkitt's lymphoma cell line with c-Myc chromosomal translocation, with a.) the Hallmark_Myc_Targets_V1, b.) Schuhmacher_Myc_targets_up, and c) Dang_Myc_Targets_Up.

FIG. 25 shows the effect of 100 nM Compound 2 for 24 hours on different MYC gene sets (obtained from the Broad Institute: http://software.broadinstitute.org/gsea/index.jsp), as measured via mRNAseq in HeLa, a cervical cancer cell line without c-Myc chromosomal translocation, with a.) the Hallmark_Myc_Targets_V1, b.) Schuhmacher_Myc_targets_up, and c) Dang_Myc_Targets_Up.

### Structural Alterations of the MYC/MYCN/MYCL Loci

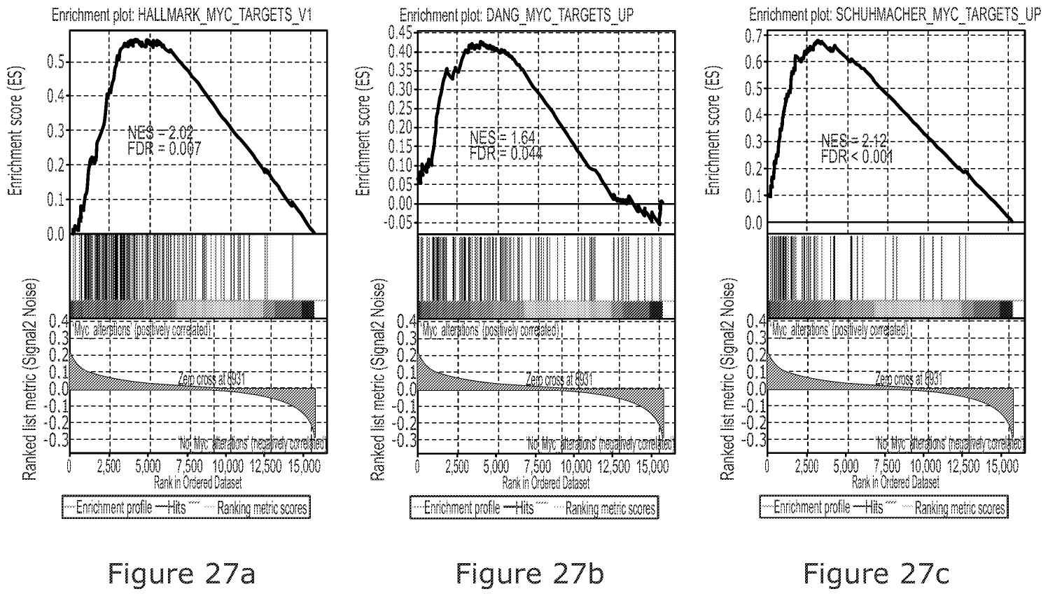

As can be seen in FIG. 26, structural alteration of MYC/MYCN/MYCL loci render cells more sensitive to the NMT inhibitor compound. As can be seen in FIG. 27, those structural alterations correlate with increased activation of different MYC gene sets.

Across the three tested cancer cell line screens, it was observed that cell lines that have structural alterations (that is: copy number gains and/or chromosomal rearrangements and/or mutations in c-MYC/MYCN/MYCL) are more sensitive to an NMT inhibitor than cell lines without such structural alterations. In two of these screens the correlation is statistically significant and in the other screen a strong trend is observed.

FIG. 26 shows: a.) the effect of the presence of structural alterations (that is: copy number gains and/or chromosomal rearrangements and/or mutations in c-MYC/MYCN/MYCL) on the sensitivity to the NMT inhibitor Compound 4 (a nonparametric Mann Whitney test was used); b.) the effect of the presence of structural alterations (that is: copy number gains and/or chromosomal rearrangements and/or mutations in c-MYC/MYCN/MYCL) on the sensitivity to the NMT inhibitor Compound 3 (a nonparametric Mann Whitney test was used); and c.) the effect of the presence of structural alterations (that is: copy number gains and/or chromosomal rearrangements and/or mutations in c-MYC/MYCN/MYCL) on the sensitivity to the NMT inhibitor Compound 6 (a nonparametric Mann Whitney test was used).

FIG. 27 shows the effect of the presence of structural alterations (that is: copy number gains and/or chromosomal rearrangements and/or mutations in c-MYC/MYCN/MYCL) on different MYC gene sets (the gene sets obtained from the Broad Institute: http://software.broadinstitute.org/gsea/index.jsp: the expression data was obtained from Iorio et. al., Cell, 2016), with a.) the Hallmark_Myc_Targets_V1, b.) Dang_Myc_Targets_Up, and c) Schuhmacher_Myc_targets_up.

### ‘Sensitive to NMTi’ Gene Set and its Appliance to MYC Driven Cancers

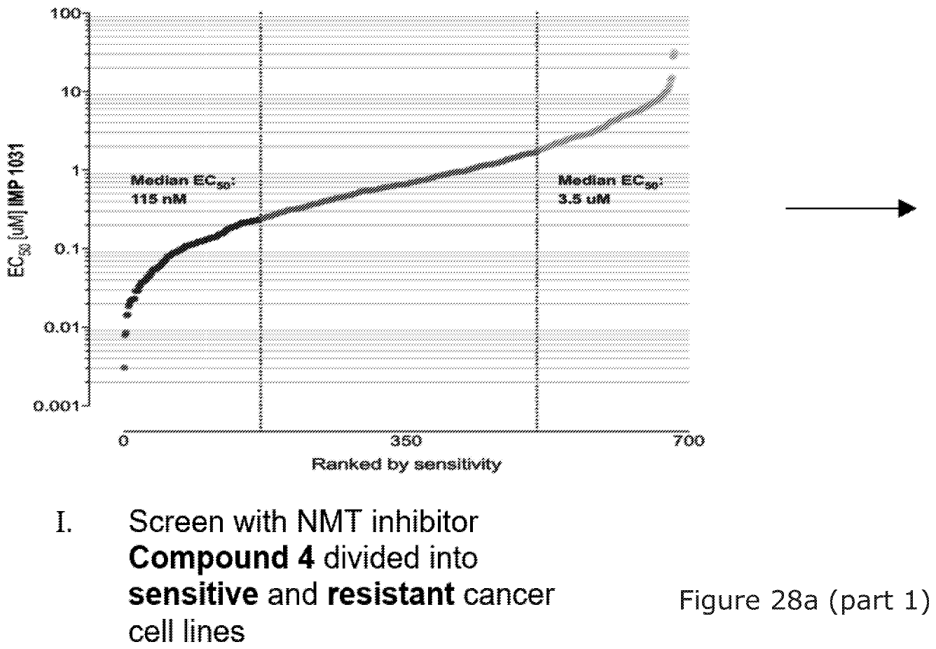

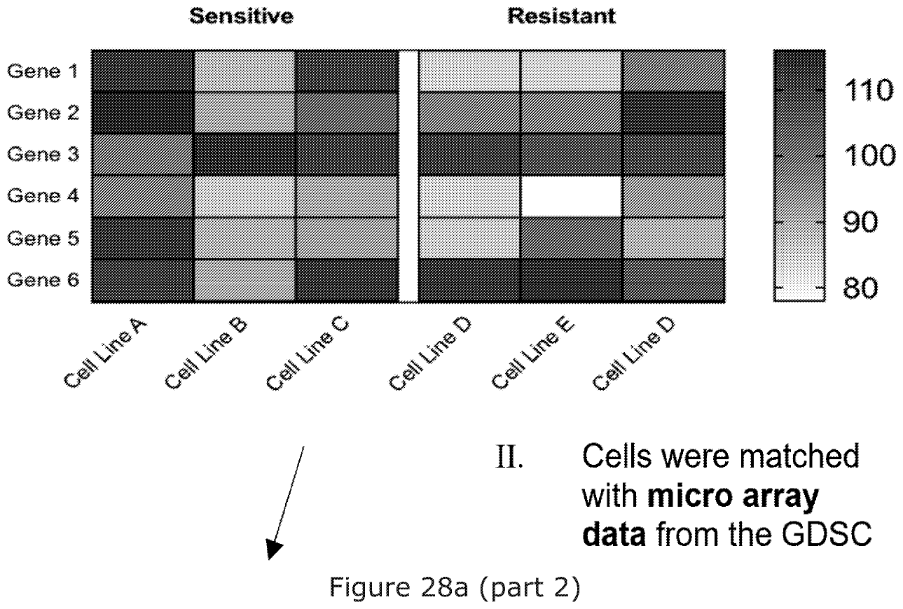

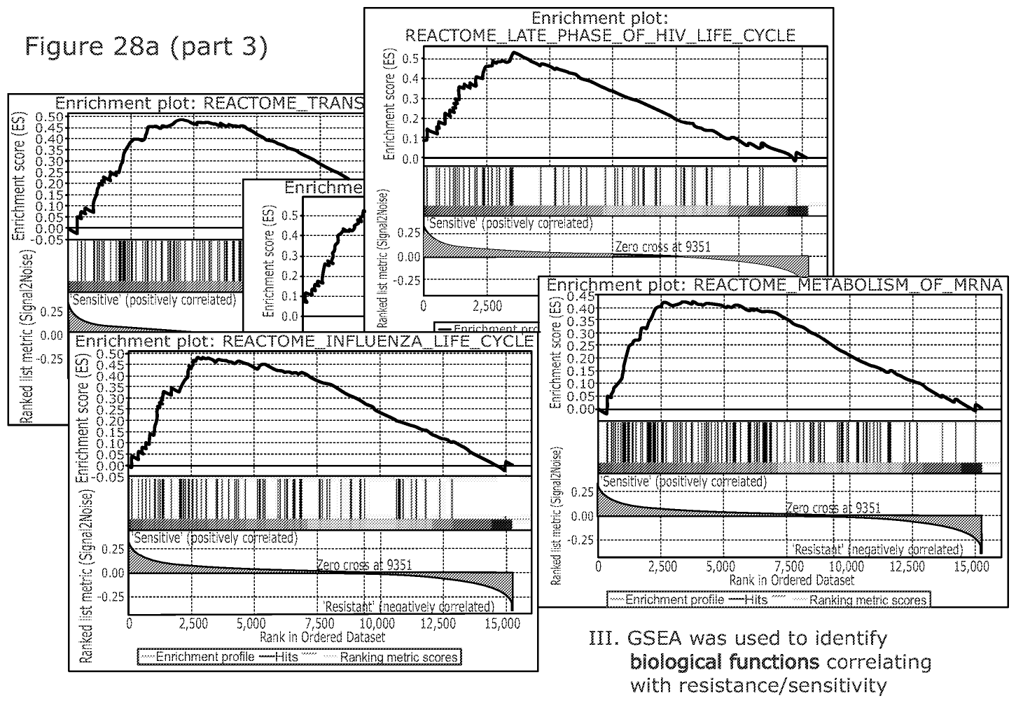

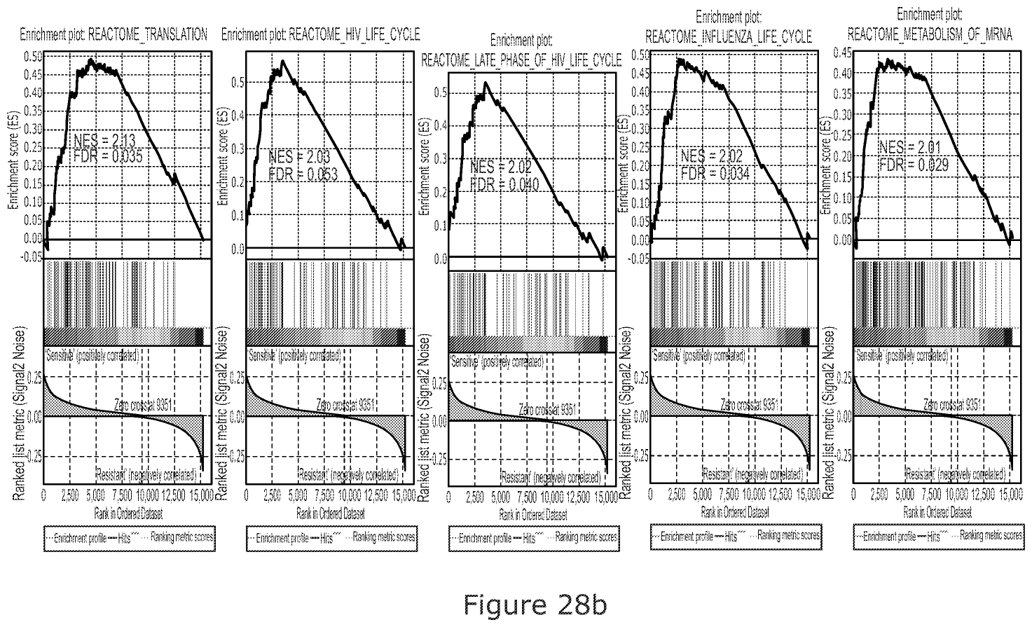

As can be seen in FIG. 28, the sensitivity to the NMT inhibitor Compound 4 can be pinpointed to certain key biological functions via GSEA: translation, transcription, protein degradation and nuclear trafficking, which are nuclear pore genes.

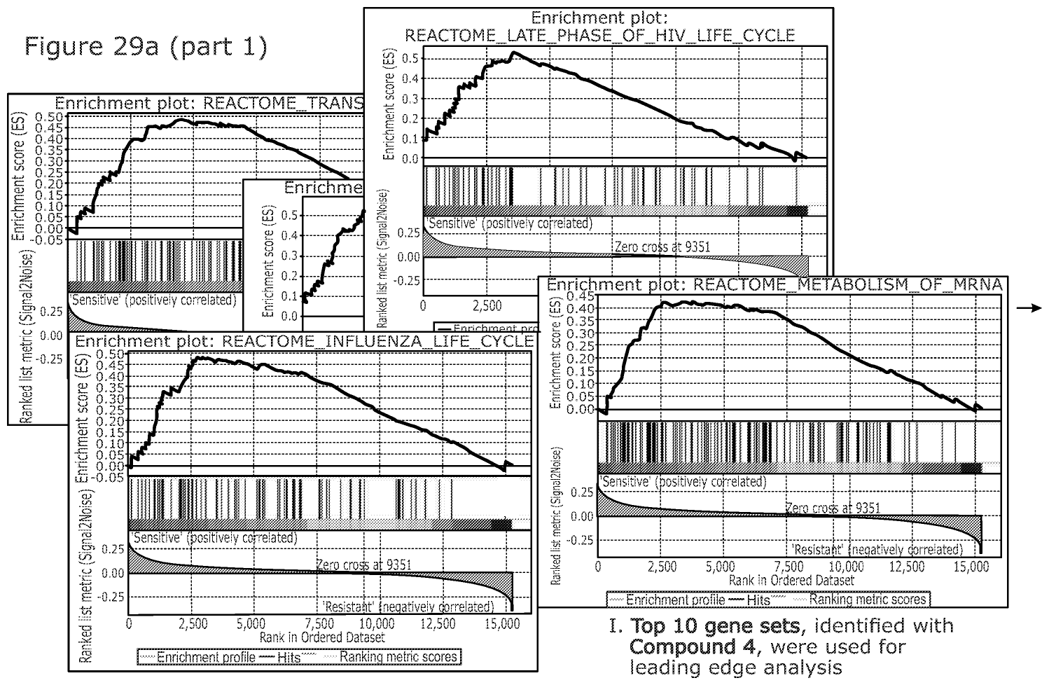

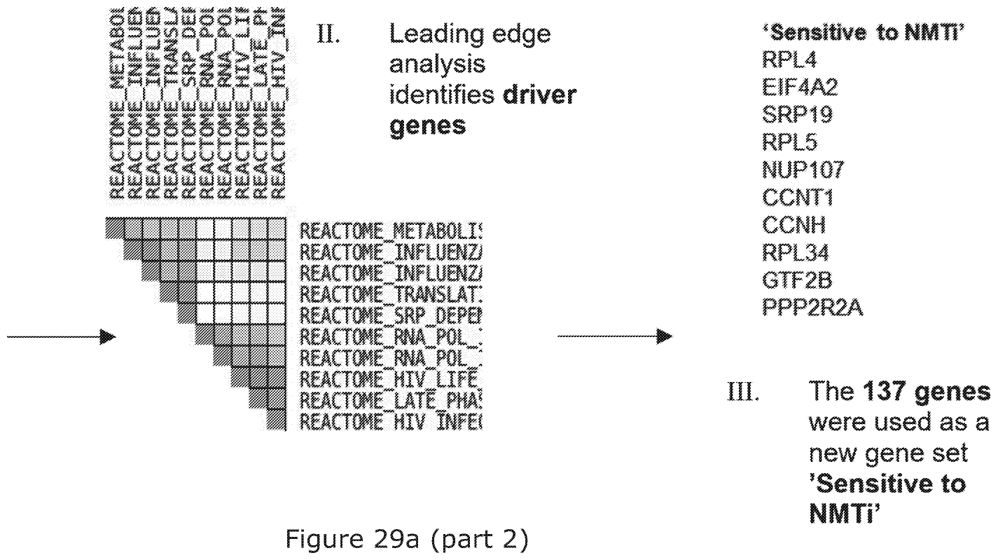

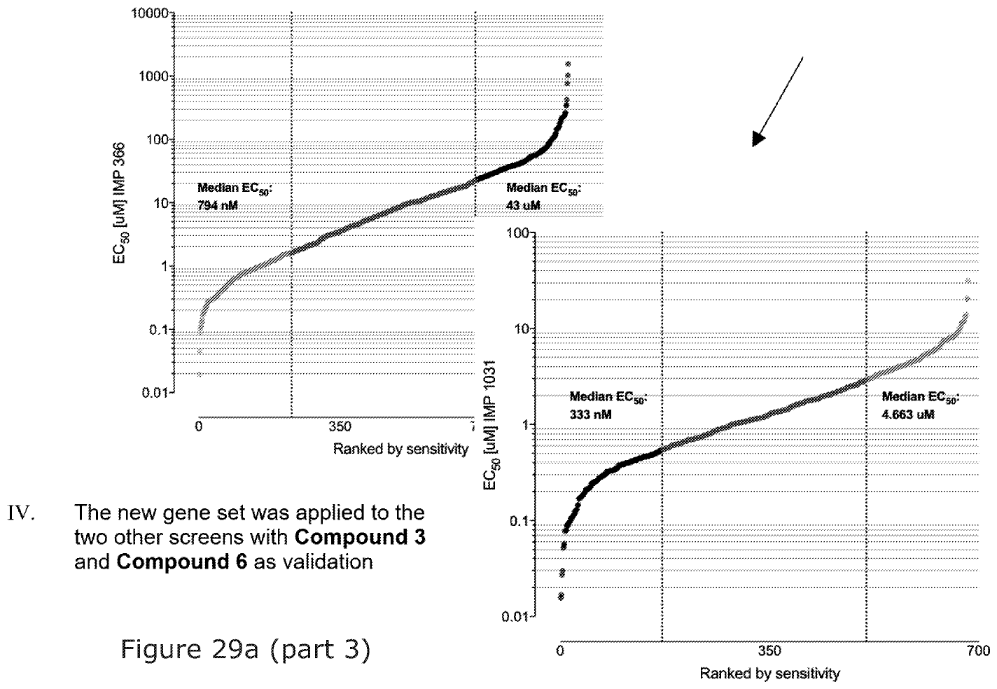

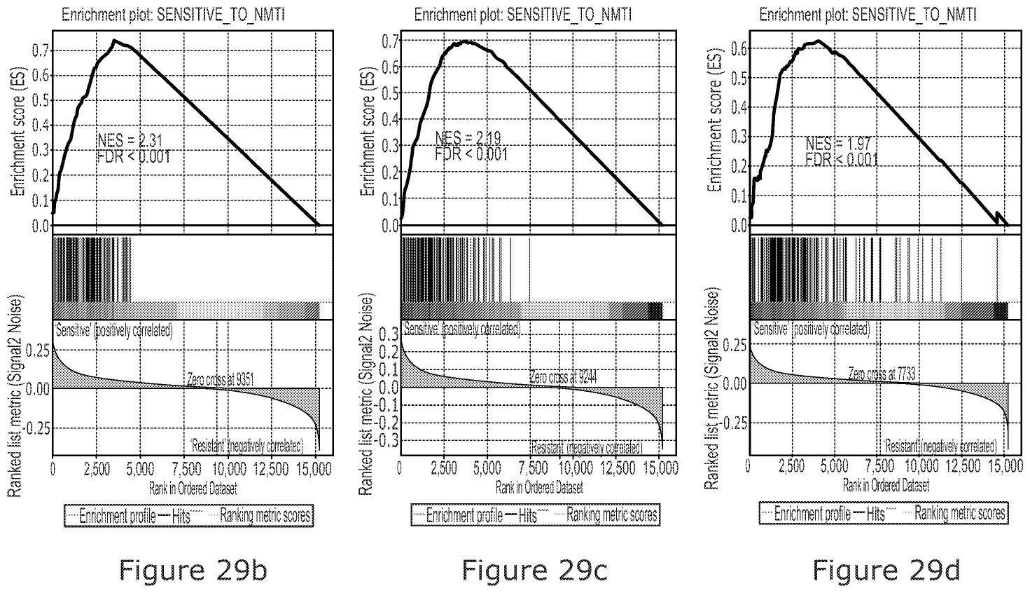

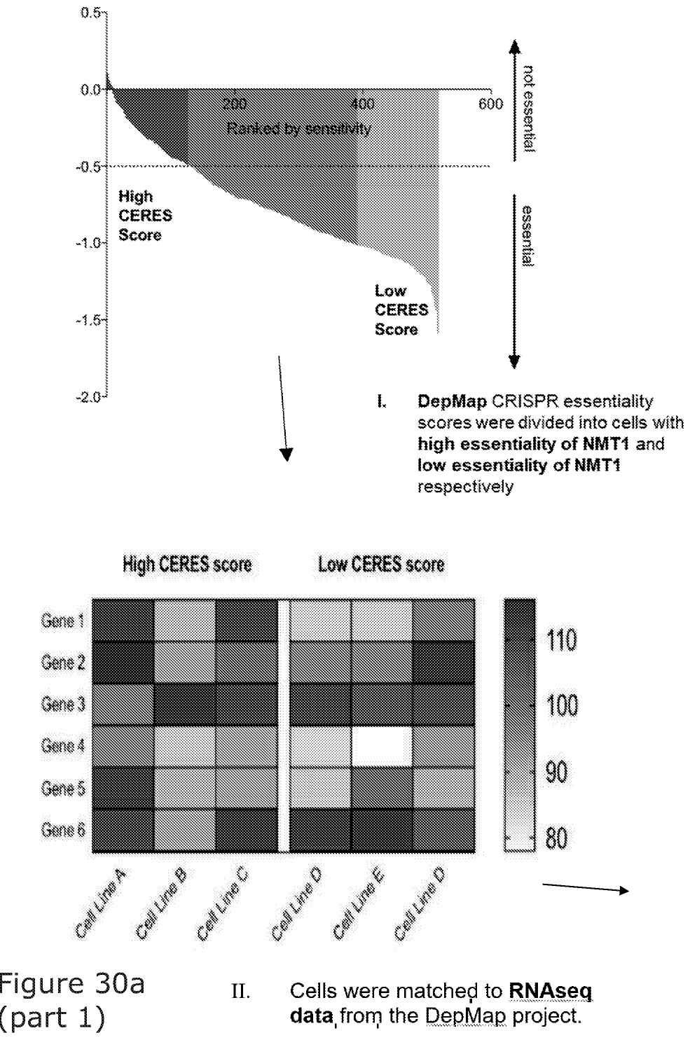

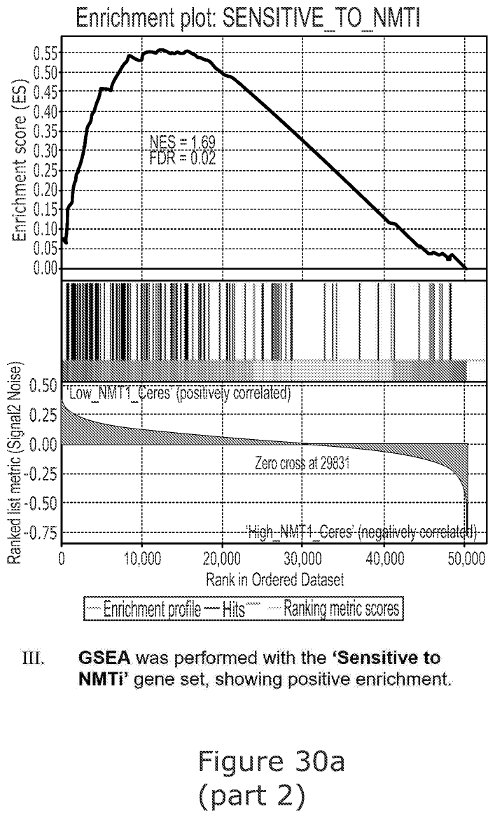

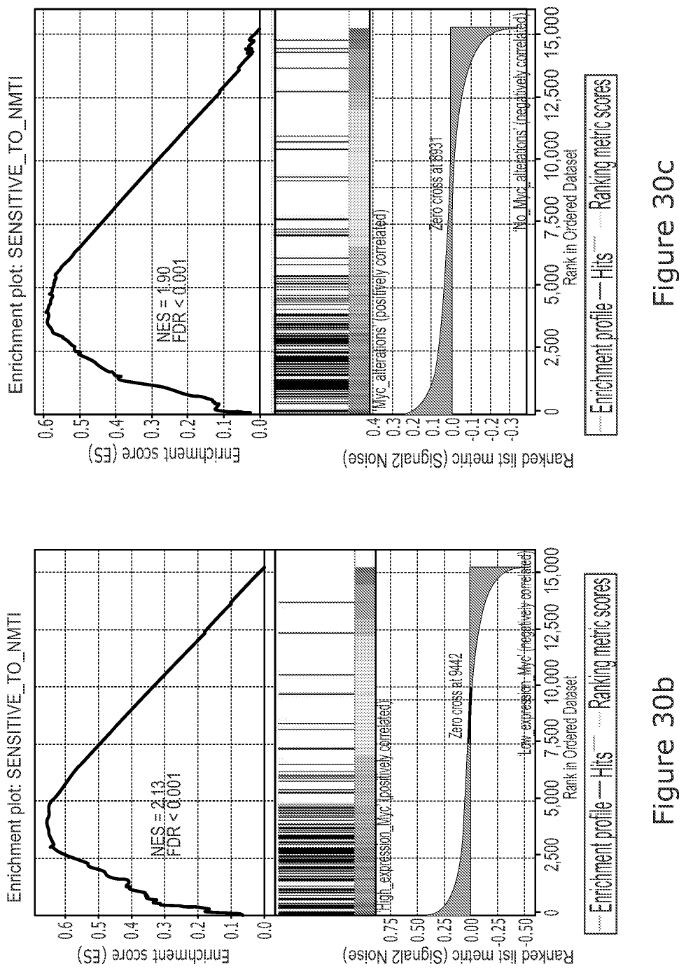

As can be seen in FIG. 29, a leading edge analysis of the top gene sets being enriched in the sensitive cancer cell lines to the NMT inhibitor Compound 4 yields in a “Sensitive to NMTi’ gene set, that contains 137 genes. This gene set is strongly enriched in the sensitive cancer cell lines to the NMT inhibitors Compound 3, Compound 4 and Compound 6, showing its predictive value. Additionally, as can be seen in FIG. 30, the ‘Sensitive to NMTi’ gene set is also enriched in cancer cell lines that are more dependent on NMT1 in a gene knock out essentiality screen. Importantly, the gene set ‘Sensitive to NMTi’ is also enriched in cells with high c-Myc expression and cancer cell lines with structural alterations in the MYC loci.

FIG. 28 shows: a.) the workflow to identify biological functions that correlate with resistance or sensitivity to the NMT inhibitor Compound 4.1. The cancer cell line screen was divided in the quartiles of the most resistant and the most responsive cell lines; II. The cell lines were matched to the expression data, obtained from Lorio et. al., Cell, 2016, 166(3), 740-754; III. GSEA was performed with the curated gene sets (C2; obtained from the Broad Institute: http://software.broadinstitute.org/gsea/index.jsp): and b.) the Top 5 gene sets by normalised enrichment score (NES), correlating with sensitivity to the NMT inhibitor Compound 4, were identified as being enriched with genes that are involved in translation, viral infection gene sets (containing genes involved in translation, transcription and protein degradation) and mRNA processing.

FIG. 29 shows: a.) the workflow to define a new gene set ‘Sensitive to NMTi’, based on the data with the NMT inhibitor Compound 4. I. The top 10 gene sets, correlating with sensitivity to the NMT inhibitor Compound 4 were used for a leading edge analysis; II. The leading edge analysis allows for the identification of driver genes in each gene set (that is: genes that cause strong enrichment of the respective gene set); III. The leading edge analysis yielded in 137 genes that were used to define a new gene set ‘Sensitive to NMTi’; IV. The newly defined gene set was applied to the screens with the NMT inhibitors Compound 1 and Compound 3 to validate the gene set; b.) The gene set ‘Sensitive to NMTi’ was applied to the cancer cell screen with the NMT inhibitor Compound 4 and shows as expected strong enrichment in the sensitive cell lines; and c.) The gene set ‘Sensitive to NMTi’ was applied to the cancer cell line screens with the NMT inhibitors Compound 3 (left) and Compound 6 (right), and shows for both NMT inhibitors strong enrichment in the sensitive cell lines respectively.

FIG. 30 shows: a.) the application of the ‘Sensitive to NMTi’ gene set to the CRISPR essentiality screening, conducted by the Broad Institute. I. The DepMap CRISPR essentiality scores were obtained from the Broad Institute (Robin, Nat Genetics, 2017, 49(12), 1779-1784; the Q4 2018 data sets are available here: https://depmap.org/portal/download/), and the cancer cell lines were divided into cell lines with high essentiality of NMT1 and low essentiality of NMT1, according to their CERES scores; II. The cell lines were matched to the RNAseq data from the Broad Institute (CCLE and GDSC Consortium, Nature, 2015, 528, 84-87; the Q4 2018 data sets are available here: https://depmap.org/portal/download/); III. The gene set ‘Sensitive to NMTi’ was applied to the data, showing enrichment in the cells with high essentiality of NMT1; b.) the ‘Sensitive to NMTi’ gene set was applied to compare cancer cell lines with high and low expression of c-MYC showing strong enrichment in the cells with high c-MYC expression; and c.) the ‘Sensitive to NMTi’ gene set was applied to compare cancer cell lines with and without structural alterations in the MYC loci, showing strong enrichment in the cancer cell lines with structural alterations in the MYC loci.

While specific embodiments of the invention have been described herein for the purpose of reference and illustration, various modifications will be apparent to a person skilled in the art without departing from the scope of the invention as defined by the appended claims.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents, patent applications and references mentioned throughout the specification of the present invention are herein incorporated in their entirety by reference.

1. A method for the treatment of a cancer, wherein said cancer comprising one or more structural alterations of the MYC locus in a subject in need of such treatment, said method comprising administering a therapeutically effective amount of a NMT inhibitor, or a pharmaceutically acceptable salt, solvate or hydrate thereof.

2. The method according to claim 1, wherein the one or more structural alterations are selected from mutations, copy-number gains and/or chromosomal rearrangements.

3. The method according to claim 1, wherein the one or more structural alterations are mutations.

4. The method according to claim 1, wherein the one or more structural alterations are mutations which impart overexpression of MYC.

5. The method according to claim 4, wherein the cancer comprises one or more mutations which impart overexpression of c-MYC and/or MYCN or wherein the one or more structural alterations are mutations which impart stabilisation of MYC.

6-8. (canceled)

9. The method according to claim 1, wherein the cancer comprising one or more structural alterations of the MYC locus is selected from a haematologic malignancy or a solid-tumour.

10. The method according to claim 9, wherein the haematologic malignancy is selected from a lymphoma, a myeloma and a leukaemia.

11. (canceled)

12. The method according to claim 1, wherein the cancer comprising one or more structural alterations of the MYC locus is selected from high grade mantle zone lymphoma, follicular lymphoma, plasmablastic lymphoma, diffuse large B-cell lymphoma and Burkitt's lymphoma.

13. The method according to claim 1, wherein the cancer comprising one or more structural alterations of the MYC locus is a myeloma or a leukaemia.

14. The method according to claim 13, wherein the myeloma is a multiple myeloma and the leukaemia is selected from chronic lymphocytic leukaemia, acute myeloid leukemia and B-acute lymphocytic leukaemia

15-16. (canceled)

17. The method according to claim 1, wherein the cancer comprising one or more structural alterations of the MYC locus is a blastoma (e.g. a neuroblastoma, a retinoblastoma or a glioblastoma).

18. The method according to claim 1, wherein the cancer comprising one or more structural alterations of the MYC locus is selected from a neuroblastoma, a retinoblastoma, a glioblastoma, a small cell lung carcinoma and an astrocytoma.

19. The method according to claim 1, wherein the cancer comprising one or more structural alterations of the MYC locus is selected from a solid tumour in an organ selected from lung, breast, prostate ovary, colon, kidney and liver; or wherein the cancer comprising one or more structural alterations of the MYC locus is a breast cancer (e.g. triple negative breast cancer or a breast invasive carcinoma).

20. (canceled)

21. The method according to claim 1, wherein the cancer comprising one or more structural alterations of the MYC locus is a solid tumour selected from ovarian serous cystadenocarcinoma, esophageal carcinoma, lung squamous cell carcinoma, lung adenocarcinoma, bladder urothelial carcinoma, uterine carcinosarcoma, stomach adenocarcinoma, breast invasive carcinoma and liver hepatocellular carcinoma; or wherein the cancer comprising one or more structural alterations of the MYC locus is selected from a multiple myeloma, a neuroblastoma, acute myeloid leukaemia, a B-acute lymphocytic leukaemia or a triple negative breast cancer (e.g. a basal-like breast cancer).

22. (canceled)

23. The method according to claim 1, wherein the NMT inhibitor is a compound of Formula I shown below, or a pharmaceutically acceptable salt, hydrate or solvate thereof: 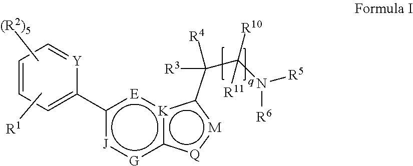
wherein:
Y is selected from the group consisting of —CH—, —C(R²)— and —N—;
R¹ is a group of formula —X-L-A;
wherein:
X is selected from the group consisting of —O—, —N(H)— and —S—, or is absent;
L is selected from the group consisting of —(CHR¹²)ₘ— and —(CHR¹²)ₘO—, or is absent;
m is 1, 2 or 3; and
A is a 6-10-membered aromatic carbocycle or a 5-10-membered aromatic heterocycle, said aromatic carbocycle or heterocycle being optionally substituted with 1, 2, or 3 substituents each independently selected from the group consisting of —F, —Cl, —Br, —OCH₃, —OCF₃, —CN, —C₁₋₆alkyl optionally substituted by up to 3 halogen, hydroxyl, or —OC₁₋₄alkyl groups, —S(O)C₁₋₄alkyl, —S(O)₂C₁₋₄alkyl, —C(O)N(R⁹)₂, —C(O)N(R¹³)C₁₋₄alkylOC₁₋₄alkyl, —C(O)N(C₁₋₄alkylOC₁₋₄alkyl)₂, —CH₂C(O)N(R⁹)₂, —CH₂C(O)N(R¹³)C₁₋₄alkylOC₁₋₄alkyl, —CH₂C(O)N(C₁₋₄alkylOC₁₋₄alkyl)₂, —S(O)₂NHC₁₋₄alkyl, —S(O)₂N(C₁₋₄alkyl)₂, —NHC₁₋₄alkyl, —N(C₁₋₄alkyl)₂, —NHC(O)C₁₋₄alkyl, —NHC(O)CF₃, —NHS(O)₂C₁₋₄alkyl, CH₂N(R¹³)₂, CH₂N(R¹³)C(O)C₁₋₄alkyl, CH₂N(R¹³)S(O)₂C₁₋₄alkyl, —CH₂S(O)₂C₁₋₄alkyl, and CO₂H;
s is 0, 1, 2, or 3;
each R² is independently selected from the group consisting of —F, —Cl, —Br, —OCH₃, —OCF₃, —CN, —C₁₋₄alkyl optionally substituted by up to 3 halogen or hydroxyl groups, —S(O)C₁₋₄alkyl, —S(O)₂C₁₋₄alkyl, —S(O)₂NHC₁₋₄alkyl, —S(O)₂N(C₁₋₄alkyl)₂, —NHC₁₋₄alkyl, —N(C₁₋₄alkyl)₂, —NHC(O)C₁₋₄alkyl, —NHC(O)CF₃, and —NHS(O)₂C₁₋₄alkyl;
E, J and G are each independently nitrogen or C(R⁷);
K is carbon or nitrogen;
and wherein:
i) when K is carbon, either Q is N(R⁸) and M is nitrogen or C(R⁷), or Q is nitrogen and M is N(R⁸); or
ii) when K is nitrogen, Q is nitrogen or C(R⁷) and M is nitrogen or C(R⁷);
and further wherein at least 2 of E, J, G, K, Q and M are selected from the group consisting of carbon and C(R⁷);
q is 0 or 1;
R³ is hydrogen or methyl; R⁴ is hydrogen or methyl;
R⁵ is hydrogen or C₁₋₆alkyl optionally substituted by up to 3 —F, —Cl, —Br, —OH, —OCH₃, —OCF₃ or —CN groups;
R⁶ is hydrogen or C₁₋₆alkyl optionally substituted by up to 3 —F, —Cl, —Br, —OH, —OCH₃, —OCF₃ or —CN groups;
or the R⁵ and R⁶ groups and the N they are bonded to form a 4 to 7 membered non-aromatic heterocycle, the heterocycle optionally comprising 1 or 2 further heteroatoms selected from N, O and S, optionally substituted by up to 3 —F, —Cl, —Br, —OH, —OCH₃, —OCF₃ or —CN groups;
when present R¹⁰ is hydrogen or methyl;
when present R¹¹ is hydrogen or methyl;
or the R³ group and the R⁵ group and the intervening atoms form a 3 to 7 membered non-aromatic heterocycle composed of the intervening atoms and bond, or the intervening atoms and —(CHR^{a})ᵣ—;
or the R¹⁰ group and the R⁵ group and the intervening atoms form a 3 to 7 membered non-aromatic heterocycle composed of the intervening atoms and —(CHR^{a})ᵣ—;
r is 1, 2, 3, 4 or 5; R^{a} is hydrogen or methyl;
each R⁷ is independently selected from the group consisting of hydrogen, halogen, C₁₋₄alkoxy, and C₁₋₄alkyl optionally substituted with 1, 2 or 3 halogens; and
R⁸ is selected from the group selected from hydrogen and C₁₋₄alkyl;
each R⁹ is independently selected from the group consisting of hydrogen and C₁₋₄alkyl, or two R⁹ groups and the N they are bonded to form a 4 to 7 membered non-aromatic heterocycle, the heterocycle optionally comprising 1 or 2 further heteroatoms selected from N, O and S;
each R¹² is independently selected from the group consisting of hydrogen, C₁₋₆alkyl optionally substituted by up to 3 —F, —Cl, —Br, I, —OH, —OCH₃, —OCF₃ or —CN groups, C₁₋₆alkenyl optionally substituted by up to 3 —F, —Cl, —Br, I, —OH, —OCH₃, —OCF₃ or —CN groups, and C₁₋₆alkynyl optionally substituted by up to 3 —F, —Cl, —Br, I, —OH, —OCH₃, —OCF₃ or —CN groups; and each R¹³ is independently selected from the group consisting of hydrogen and C₁₋₄alkyl.

24. The method according to claim 1, wherein the NMT inhibitor is a compound of Formula (IA\hat{ }\hat{ }) shown below, or a pharmaceutically acceptable salt, hydrate or solvate thereof: 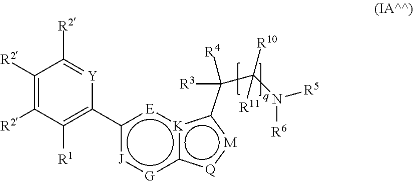
wherein:
R¹ is a group of formula —X-L-A;
A is 4-pyrazolyl, said pyrazolyl being optionally substituted with up to 3 methyl groups;
X is —O— or absent;
L is —(CH₂)ₘ— or —(CH₂)ₘ—O—;
m is 2;
R^{2′} is selected from the group consisting of fluorine, chlorine —CN and methyl (preferably fluorine);
R^{2″} is selected from the group consisting of hydrogen, fluorine, chlorine, —CN and methyl;
q is 0 or 1;
R³ is hydrogen or methyl;
R⁴ is hydrogen or methyl;
R⁵ is hydrogen or methyl;
R⁶ is hydrogen or methyl; or
the R³ group and the R⁵ group and the intervening atoms form a 3 to 7 membered non-aromatic heterocycle composed of the intervening atoms and bonds, (more preferably R⁵ and R⁶ are both methyl);
R⁷ where present is hydrogen or methyl;
R⁸ where present is hydrogen or methyl;
R⁹ and R¹⁰ where present are hydrogen or methyl; and
E, J, G, K, Q and M are:
i) E, J and G are each C(R⁷), K is carbon, Q is N(R⁸), M is nitrogen; and R⁸ is hydrogen or methyl;
ii) E, J and G are each C(R⁷), and K, Q and M are each nitrogen;
iii) E and G are each C(R⁷), and J, K, Q and M are each nitrogen;
iv) J and G are each C(R⁷), and E, K, Q and M are each nitrogen; or
v) E, J, G and M are each C(R⁷), and K and Q are each nitrogen.

25. The method according to claim 1, wherein the NMT inhibitor is a compound of Formula (IA\hat{ }\hat{ }) shown below, or a pharmaceutically acceptable salt, hydrate or solvate thereof: 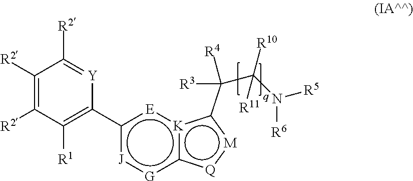
wherein:
R¹ is a group of formula —X-L-A;
A is 4-pyrazolyl, said pyrazolyl being optionally substituted with up to 3 substituent groups selected from methyl and —C(O)N(CH₃)₂;
X is —O— or absent;
L is —(CH₂)ₘ— or —(CH₂)ₘ—O—;
m is 2;
R^{2′} is selected from the group consisting of fluorine or chlorine (preferably fluorine);
R^{2″} is selected from the group consisting of hydrogen, fluorine or chlorine;
q is 0;
R³ is hydrogen or methyl;
R⁴ is hydrogen or methyl;
R⁵ is hydrogen or methyl;
R⁶ is hydrogen or methyl; or
the R³ group and the R⁵ group and the intervening atoms form a 3 to 7 membered non-aromatic heterocycle composed of the intervening atoms and bonds, (more preferably R⁵ and R⁶ are both methyl);
E, J, G, K, Q and M are:
i) E, J and G are each CH, K is carbon, Q is N(R⁸), M is nitrogen; and R⁸ is hydrogen or methyl; or
ii) E, J, G and M are each CH, and K and Q are each nitrogen;
with the proviso that A is substituted with no more than one —C(O)N(CH₃)₂ group.

26. The method according to claim 1, wherein the NMT inhibitor is a compound selected from: 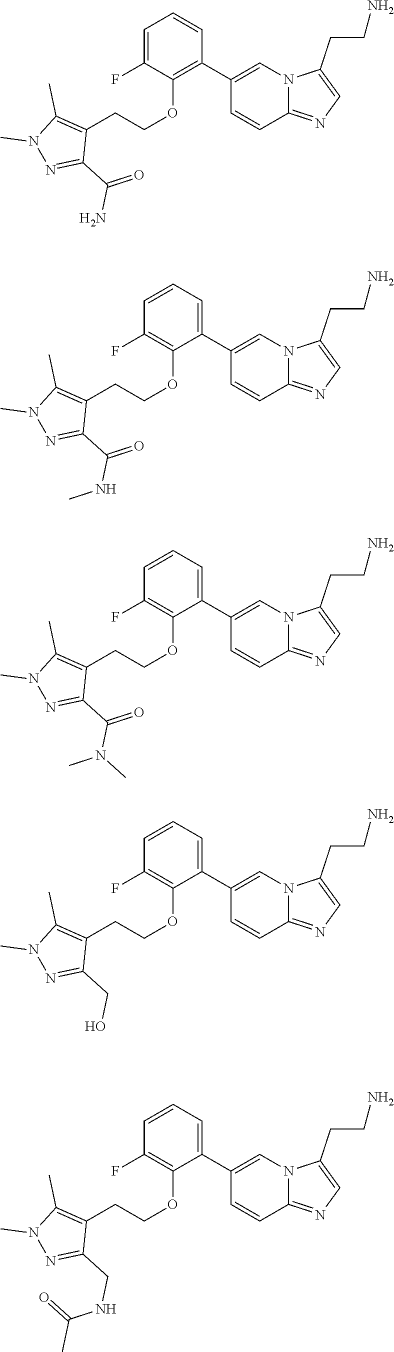 [SMILES: Cc1c(CCOc2c(F)cccc2-c2ccc3ncc(CCN)n3c2)c(C(N)=O)nn1C ; CNC(=O)c1nn(C)c(C)c1CCOc1c(F)cccc1-c1ccc2ncc(CCN)n2c1 ; Cc1c(CCOc2c(F)cccc2-c2ccc3ncc(CCN)n3c2)c(C(=O)N(C)C)nn1C ; Cc1c(CCOc2c(F)cccc2-c2ccc3ncc(CCN)n3c2)c(CO)nn1C ; CC(=O)NCc1nn(C)c(C)c1CCOc1c(F)cccc1-c1ccc2ncc(CCN)n2c1] 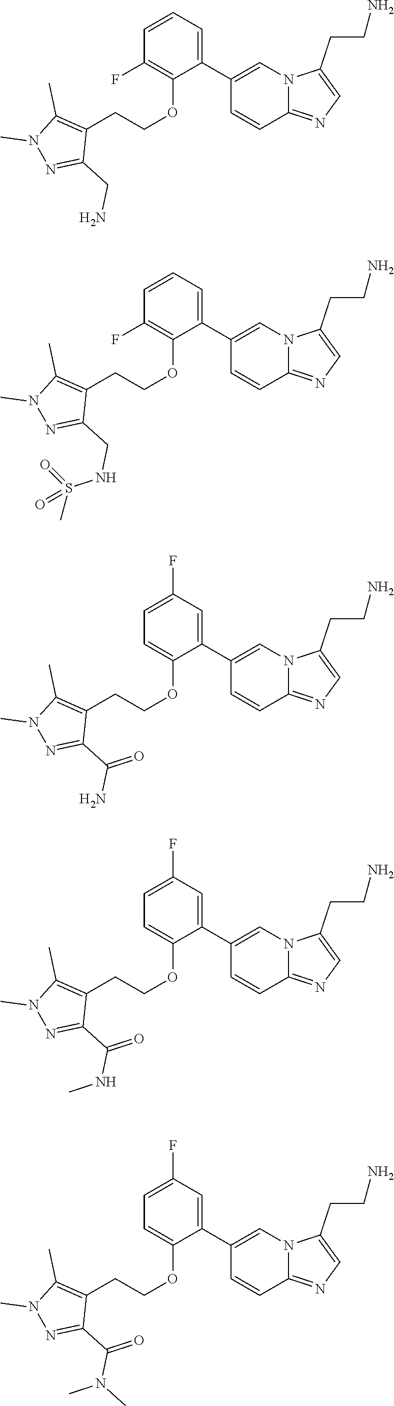 [SMILES: Cc1c(CCOc2c(F)cccc2-c2ccc3ncc(CCN)n3c2)c(CN)nn1C ; Cc1c(CCOc2c(F)cccc2-c2ccc3ncc(CCN)n3c2)c(CNS(C)(=O)=O)nn1C ; Cc1c(CCOc2ccc(F)cc2-c2ccc3ncc(CCN)n3c2)c(C(N)=O)nn1C ; CNC(=O)c1nn(C)c(C)c1CCOc1ccc(F)cc1-c1ccc2ncc(CCN)n2c1 ; Cc1c(CCOc2ccc(F)cc2-c2ccc3ncc(CCN)n3c2)c(C(=O)N(C)C)nn1C] 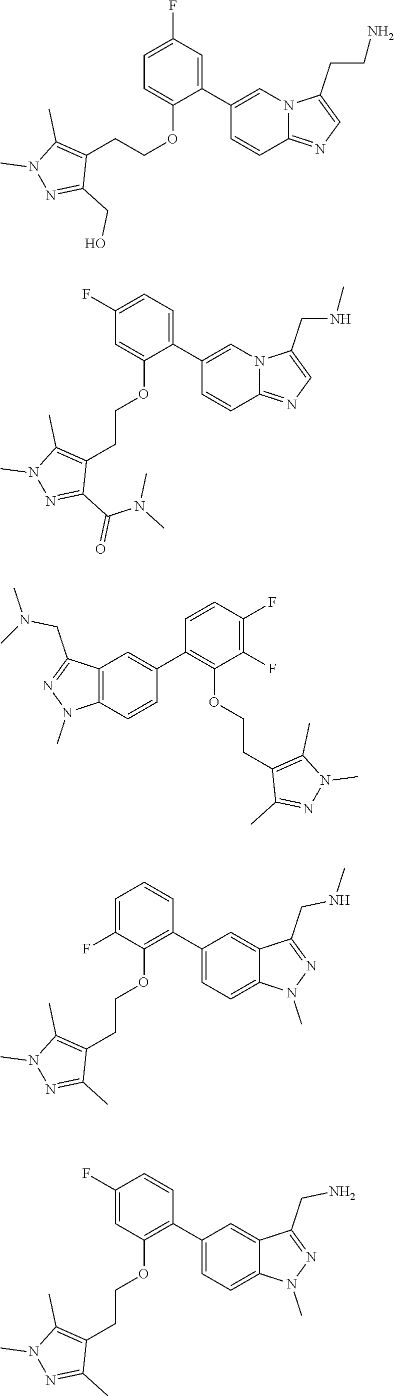
or a pharmaceutically acceptable salt, hydrate or solvate thereof.

27. (canceled)

28. The method according to claim 1, wherein the NMT inhibitor is a compound of Formula (Id) shown below, or a pharmaceutically acceptable salt, hydrate or solvate thereof: 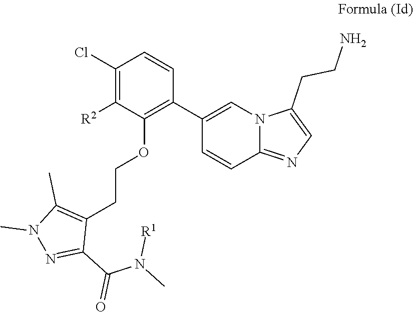
wherein:
R¹ is H or —CH₃; and
R² is H or F.

29. The method according to claim 28, wherein the NMT inhibitor is the compound shown below, or a pharmaceutically acceptable salt, hydrate or solvate thereof:

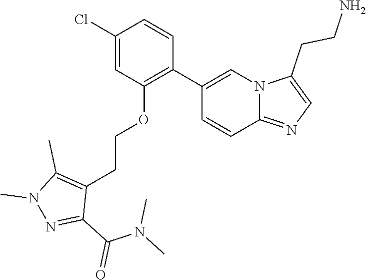 [SMILES: Cc1c(CCOc2cc(Cl)ccc2-c2ccc3ncc(CCN)n3c2)c(C(=O)N(C)C)nn1C]

30. The method according to claim 1, wherein the NMT inhibitor is a compound of Formula (II) or Formula (III) shown below, or a pharmaceutically acceptable salt, hydrate or solvate thereof: 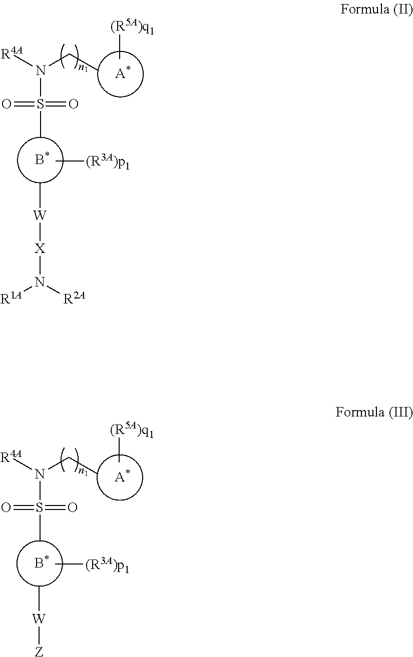
wherein:
m is 0, 1, 2, 3, 4, 5 or 6;
Ring A*, is an optionally substituted nitrogen containing aryl group wherein each substitutable carbon or nitrogen in Ring A* is optionally and independently substituted by one or more R^{5A} and wherein if Ring A* contains an —NH— moiety that nitrogen may be optionally substituted by C₁₋₆alkyl (e.g. methyl); and wherein R^{4A} and Ring A* together with the atoms to which they are attached may form a cyclic group,
Ring B* is an optionally substituted aryl or heteroaryl group wherein each substitutable carbon or heteroatom in Ring B* is optionally and independently substituted by one or more R^{3A};
W and X, one of which may be absent, are independently selected from R^{11A}, hydrocarbyl (e.g. C₁₋₈ alkyl, alkenyl, alkynyl, or haloalkyl) optionally substituted with R^{11A}, and —(CH₂)ₖ₁-heterocyclyl optionally substituted with R^{12A}; k₁ is 0, 1, 2, 3, 4, 5 or 6;
R^{1A}, R^{2A}, R^{3A}, R^{4A} and R^{5A} are independently selected from hydrogen, R^{12A}, hydrocarbyl (e.g. C₁₋₆ alkyl, alkenyl, alkynyl, or haloalkyl) optionally substituted with R^{12A}, and a —(CH₂)_{L1}-heterocyclyl optionally substituted with one or more R^{12A}; wherein R^{1A} and R^{2A} taken together with the atoms to which they are attached may form a heterocycle, optionally substituted with one or more R^{12A}, wherein R^{1A} and/or R^{2A} taken together with W or X may form a heterocycle optionally substituted with one or more R^{12A}; and wherein one or more of R^{3A} and R^{5A} taken together with the atoms to which they are attached may form a carbocycle, for example heterocyclyl, optionally substituted with R^{12A}; L₁ is 0, 1, 2, 3, 4, 5 or 6;
wherein:
each R^{11A} and R^{12A} is independently selected from halogen, trifluoromethyl, cyano, thio, nitro, oxo, ═NR^{13A}, —OR^{13A}, —SR^{13A}, —C(O)R^{13A}, —C(O)OR^{13A}, —OC(O)R^{13A}, —NR^{13A}COR^{14A}, —NR^{13A}CON(R^{13A})₂, —NR^{13a}COR^{14a}, —NR^{13a}CO₂R^{14A}, —S(O)R^{13A}, —S(O)₂R^{13A}, —SON(R^{13A})₂, —NR^{13A}S(O)₂R^{14A}; —CSR^{13A}, —N(R^{13A})R^{14A}, —C(O)N(R^{13A})R^{14A}, —SO₂N(R^{13A})R^{14A} and R^{15A};
R^{13A} and R^{14A} are each independently selected from hydrogen or R^{15A};
R^{15A} is selected from hydrocarbyl (e.g. C₁₋₆alkyl, alkenyl, alkynyl, or haloalkyl), carbocyclyl and —(CH₂)ₘ₁-heterocyclyl, and each R^{15A} is optionally and independently substituted with one or more of halogen, cyano, amino, hydroxy, C₁₋₆alkyl or cycloalkyl and C₁₋₆alkoxy;
m₁ is 0, 1, 2, 3, 4, 5 or 6;
p₁ is 0, 1, 2, 3 or 4; the values of R^{4A} may be the same or different; and
q₁ is 0, 1, 2, 3 or 4; wherein the values of R^{5A} may be the same or different;
Y and Z, one or both of which may be absent, are independently selected from hydrogen, R^{16A}, hydrocarbyl (e.g. C₁₋₆alkyl, alkenyl, alkynyl. or haloalkyl) optionally substituted with R^{16A}, and —(CH₂)ᵣ₁-heterocyclyl optionally substituted with R^{16A}, wherein each R^{16A} is independently selected from halogen, trifluoromethyl, cyano, thio, nitro, oxo, ═NR^{17A}, —OR^{17A}, —SR^{17A}, —C(O)R^{17A}, —C(O)OR^{17A}, —OC(O)R^{17A}, —NR^{17A}COR^{18A}, —NR^{17A}CON(R^{18A})₂, —NR^{17A}COR^{18A}, —NR^{17A}CO₂R^{18A}, —S(O)R^{17A}, —S(O)₂R^{17A}, —SON(R^{17A})₂, —NR^{17A}S(O)₂R^{18A}; —CSR^{17A}, —N(R^{17A})R^{18A}, —C(O)N(R^{17A})R^{18A}, —SO₂N(R^{17A})R^{18A} and R^{19A}; n is 0, 1, 2, 3, 4, 5 or 6;
wherein:
R^{17A} and R^{18A} are each independently selected from hydrogen or R^{19A};
R^{19A} is selected from hydrocarbyl (e.g. C₁₋₆alkyl, alkenyl, alkynyl. or haloalkyl), carbocyclyl and —(CH₂)ₛ₁-heterocyclyl, and each R^{19A} is optionally and independently substituted with one or more of halogen, cyano, amino, hydroxy, C₁₋₆alkyl and C₁₋₆alkoxy; and
s₁ is 0, 1, 2, 3, 4, 5 or 6.

31. The method according to claim 30, wherein the NMT inhibitor is a compound of Formula (IIa) shown below, or a pharmaceutically acceptable salt, solvate or hydrate thereof: 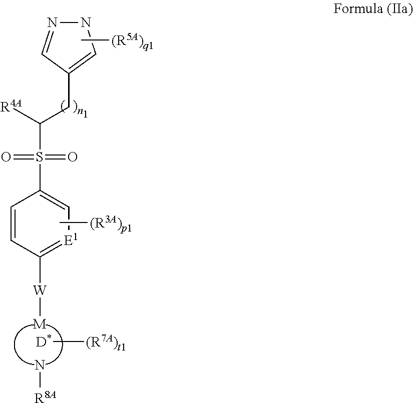
wherein:
n₁ is 0 or 1;
E¹ is C;
W is a (1-4C)hydrocarbyl, an aryl (e.g. phenyl) or heteroaryl group (e.g. pyridinyl);
M is selected from C and N;
R^{3A}, R^{4A} and R^{5A} are independently selected from hydrogen, R^{12A}, and (1-3C)hydrocarbyl optionally substituted with R^{12A};
R^{12A} is independently selected from halogen, trifluoromethyl, cyano, thio, nitro, oxo, —OR^{13A}, —SR^{13A}, —C(O)R^{13A}, —C(O)OR^{13A}, —OC(O)R^{13A}, —NR^{13A}COR^{14A} and R^{15A};
R^{13A} and R^{14A} are each independently selected from hydrogen or a (1-4C)hydrocarbyl (e.g. methyl);
Ring D* is an optionally substituted nitrogen containing 6 or 7 membered heterocycle, wherein each substitutable carbon or nitrogen in Ring D* is optionally and independently substituted by one or more R^{7A};
R^{7A} is independently selected from hydrogen, (1-4C)hydrocarbyl, halogen, trifluoromethyl, cyano, thio, nitro or oxo;
R^{8A} is a hydrogen or a (1-4C)hydrocarbyl (e.g. methyl);
p₁ is 0, 1 or 2, wherein the values of R^{4A} may be the same or different;
q₁ is 3, wherein the values of R^{5A} may be the same or different; and
t₁ is 0, 1 or 2, wherein the values of R^{7A} may be the same or different.

32. The method according to claim 30, wherein the NMT inhibitor is selected from the compounds shown below, or a pharmaceutically acceptable salt, hydrate or solvate thereof:

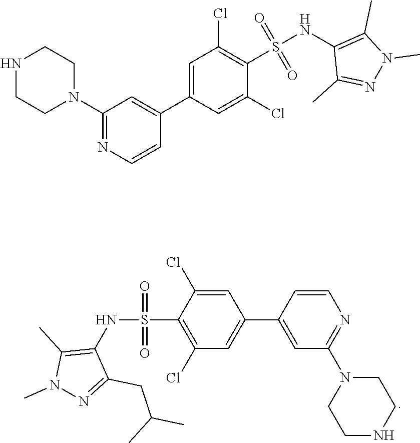

33. A method for the treatment of a cancer comprising one or more structural alterations of the MYC locus in a subject in need of such treatment said method comprising administering a therapeutically effective amount of a NMT inhibitor, or a pharmaceutically acceptable salt, solvate or hydrate thereof, in combination with one or more other therapeutic agents.

34-35. (canceled)

36. A method for the treatment of a MYC addicted cancer (e.g. a c-MYC or MYCN addicted cancer) in a subject in need of such treatment, said method comprising administering a therapeutically effective amount of a NMT inhibitor, or a pharmaceutically acceptable salt, solvate or hydrate thereof, optionally in combination with one or more other therapeutic agents.

37. A method for determining whether a subject with a cancer will benefit from treatment with an NMT inhibitor, said method comprising the steps of:
taking a sample of cancer cells taken from said subject;
analysing the cells of step i) to check for the presence of one or more structural alterations in the MYC locus (e.g. chromosomal rearrangements, copy number gains and/or mutations of the MYC oncogene);
determining whether one or more structural alterations (e.g. chromosomal rearrangements, copy number gains and/or mutations) are present in the MYC locus of the sample of cancer cells when compared to a control; and
determining whether the subject will benefit from being administered a NMT inhibitor in order to treat said cancer, wherein if the sample of cancer cells contain one or more structural alterations (e.g. chromosomal rearrangements, copy number gains and/or mutations) in the MYC locus, then the subject will benefit from being administered a NMT inhibitor, and if the sample of cancer cells do not contain one or more structural alterations (e.g. chromosomal rearrangements, copy number gains and/or mutations) in the MYC locus, then the subject will not benefit from being administered a NMT inhibitor.

38. A method for determining whether a subject with a cancer will benefit from treatment with an NMT inhibitor, said method comprising the steps of:
i) measuring the level of MYC expression in a sample of cancer cells taken from said subject;
ii) comparing the level of MYC expression from step i) with a control;
iii) determining whether the MYC expression in the sample of cancer cells is increased compared to the control; and
iv) determining whether the subject will benefit from being administered a NMT inhibitor in order to treat said cancer, wherein if the MYC expression in the sample of cancer cells is higher than in the control, then the subject will benefit from being administered a NMT inhibitor, and if the MYC expression in the sample of cancer cells is not higher than in the control, then the subject will not benefit from being administered a NMT inhibitor

39. (canceled)
